SEQUENCE LISTING (46 sequences)

SEQ ID NO: 1 - protein, 163 aa, Artificial sequence
  synthetic sequence
DYKDDDDKGSMAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGSILGILGGILALLILILLLLLFGS

SEQ ID NO: 2 - protein, 274 aa, Artificial sequence
  synthetic sequence
DYKDDDDKGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSILGILGGILALLILILLLLLFGS

SEQ ID NO: 3 - protein, 435 aa, Artificial sequence
  synthetic sequence
DYKDDDDKGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 4 - protein, 324 aa, Artificial sequence
  synthetic sequence
DYKDDDDKGSMAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGSGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 5 - protein, 311 aa, Artificial sequence
  synthetic sequence
DYKDDDDKGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGIIAAVVVVALVISVCGLGVCYAQRKGYFSKETSFQKSNSSSKATTMSENDFKHTKSFII

SEQ ID NO: 6 - protein, 200 aa, Artificial sequence
  synthetic sequence
DYKDDDDKGSMAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGSGIIAAVVVVALVISVCGLGVCYAQRKGYFSKETSFQKSNSSSKATTMSENDFKHTKSFII

SEQ ID NO: 7 - protein, 281 aa, Artificial sequence
  synthetic sequence
DYKDDDDKGSMAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGSGLSTGAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPEKGPVEAKPECQETETKPAPAEVKTVPNDATQTKENESKA

SEQ ID NO: 8 - protein, 392 aa, Artificial sequence
  synthetic sequence
DYKDDDDKGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGLSTGAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPEKGPVEAKPECQETETKPAPAEVKTVPNDATQTKENESKA

SEQ ID NO: 9 - protein, 296 aa, Artificial sequence
  synthetic sequence
DYKDDDDKGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSAFFGIFFGALGGLLLLGVGTFVVLRFWGCSGARFSYFLNSAEALP

SEQ ID NO: 10 - protein, 185 aa, Artificial sequence
  synthetic sequence
DYKDDDDKGSMAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGSAFFGIFFGALGGLLLLGVGTFVVLRFWGCSGARFSYFLNSAEALP

SEQ ID NO: 11 - protein, 303 aa, Artificial sequence
  synthetic sequence
DYKDDDDKGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSIVIITVVAAAVIMGTAGLSTYLYNRQRKIKKYRLQQAQKGTPMKPNTQATPP

SEQ ID NO: 12 - protein, 192 aa, Artificial sequence
  synthetic sequence
DYKDDDDKGSMAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGSIVIITVVAAAVIMGTAGLSTYLYNRQRKIKKYRLQQAQKGTPMKPNTQATPP

SEQ ID NO: 13 - protein, 313 aa, Artificial sequence
  synthetic sequence
DYKDDDDKGSMAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGSILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 14 - protein, 424 aa, Artificial sequence
  synthetic sequence
DYKDDDDKGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 15 - protein, 320 aa, Artificial sequence
  synthetic sequence
DYKDDDDKGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSIAAIVGGTVAGIVLIGILLLVIWKALIHLSDLREYRRFEKEKLKSQWNNDNPLFKSATTTVMNPKFAES

SEQ ID NO: 16 - protein, 209 aa, Artificial sequence
  synthetic sequence
DYKDDDDKGSMAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGSIAAIVGGTVAGIVLIGILLLVIWKALIHLSDLREYRRFEKEKLKSQWNNDNPLFKSATTTVMNPKFAES

SEQ ID NO: 17 - protein, 199 aa, Artificial sequence
  synthetic sequence
DYKDDDDKGSMAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGSLTYFGGAVASTIGLIMGGTLLALLRKRFRQKDDGKCPLNPHSHLGTYGVFTNAAFDPSP

SEQ ID NO: 18 - protein, 310 aa, Artificial sequence
  synthetic sequence
DYKDDDDKGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSLTYFGGAVASTIGLIMGGTLLALLRKRFRQKDDGKCPLNPHSHLGTYGVFTNAAFDPSP

SEQ ID NO: 19 - protein, 343 aa, Artificial sequence
  synthetic sequence
DYKDDDDKGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 20 - protein, 232 aa, Artificial sequence
  synthetic sequence
DYKDDDDKGSMAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGSLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 21 - protein, 313 aa, Artificial sequence; identical to SEQ ID NO: 13
  synthetic sequence

SEQ ID NO: 22 - protein, 424 aa, Artificial sequence; identical to SEQ ID NO: 14
  synthetic sequence

SEQ ID NO: 23 - protein, 313 aa, Artificial sequence
  synthetic sequence
DYKDDDDKGSMADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVSGSILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 24 - protein, 156 aa, Artificial sequence
  synthetic sequence
YPYDVPDYANMLDAPTDLQVTNVTDTSITVSWTPPSATITGYRITYTPSNGPGEPKELTVPPSSTSVTITGLTPGVEYVVSVYALKDNQESPPLVGTQTTGGGSIVIITVVAAAVIMGTAGLSTYLYNRQRKIKKYRLQQAQKGTPMKPNTQATPP

SEQ ID NO: 25 - protein, 313 aa, Artificial sequence
  synthetic sequence
DYKDDDDKGSMAEVQLVESGGDLVKPGGSLKLSCAASGFTFSSYGMSWVRQTPDKRLEWVATISRGGSYTYYPDSVKGRFTISRDNAKNTLYLQMSSLKSEDTAMYYCARRETYDEKGFAYWGQGTSVTVSSGGGGSGGGGSGGGGSDIVLTQSPASLAVSLGQRATISCKSSQSLLNSGNQKNYLTWYQQKPGQPPKLLIYWASTRESGIPARFSGSGSGTDFTLNIHPVEEEDAATYYCQNDNSHPLTFGGGTKLEIGSIVIITVVAAAVIMGTAGLSTYLYNRQRKIKKYRLQQAQKGTPMKPNTQATPP

SEQ ID NO: 26 - protein, 699 aa, Artificial sequence
  synthetic sequence
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGSIVIITVVAAAVIMGTAGLSTYLYNRQRKIKKYRLQQAQKGTPMKPNTQATPP

SEQ ID NO: 27 - protein, 191 aa, Artificial sequence
  synthetic sequence
EQKLISEEDLEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGSIVIITVVAAAVIMGTAGLSTYLYNRQRKIKKYRLQQAQKGTPMKPNTQATPP

SEQ ID NO: 28 - protein, 437 aa, Artificial sequence
  synthetic sequence
DYKDDDDKGSGPSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTPGSAGSIVIITVVAAAVIMGTAGLSTYLYNRQRKIKKYRLQQAQKGTPMKPNTQATPP

SEQ ID NO: 29 - protein, 190 aa, Artificial sequence
  synthetic sequence
DYKDDDDKGSSQVQLVESGGGSVQAGGSLRLSCVASGDIKYISYLGWFRQAPGKEREGVAALYTSTGRTYYADSVKGRFTVSLDNAKNTVYLQMNSLKPEDTALYYCAAAEWGSQSPLTQWFYRYWGQGTQVTVSAGSIVIITVVAAAVIMGTAGLSTYLYNRQRKIKKYRLQQAQKGTPMKPNTQATPP

SEQ ID NO: 30 - protein, 986 aa, Artificial sequence
  synthetic sequence
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGSIVIITVVAAAVIMGTAGLSTYLYNRQRKIKKYRLQQAQKGTPMKPNTQATPP

SEQ ID NO: 31 - protein, 316 aa, Artificial sequence
  synthetic sequence
DYKDDDDKGSEVDLVESGGDLVKPGGSLKLSCAASGFTFSHYGMSWVRQTPDKRLEWVATIGSRGTYTHYPDSVKGRFTISRDNDKNALYLQMNSLKSEDTAMYYCARRSEFYYYGNTYYYSAMDYWGQGTTVTVSSTTAASGSSGGSSSGDIVLTQSPASLAVSLGQRATISCRASESVDNYGFSFMNWFQQKPGQPPKLLIYAISNRGSGVPARFSGSGSGTDFSLNIHPVEEDDPAMYFCQQTKEVPWTFGAGTKLEIKGSIVIITVVAAAVIMGTAGLSTYLYNRQRKIKKYRLQQAQKGTPMKPNTQATPP

SEQ ID NO: 32 - protein, 341 aa, Artificial sequence
  synthetic sequence
YPYDVPDYAGAQPARPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKGSIVIITVVAAAVIMGTAGLSTYLYNRQRKIKKYRLQQAQKGTPMKPNTQATPP

SEQ ID NO: 33 - protein, 306 aa, Artificial sequence
  synthetic sequence
DYKDDDDKGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSIVIITVVAAAVIMGTAGLSTYLYNRQRKIKKYRLQQAQKGTPMKPNTQATPP

SEQ ID NO: 34 - protein, 300 aa, Artificial sequence
  synthetic sequence
DYKDDDDKGSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSIVIITVVAAAVIMGTAGLSTYLYNRQRKIKKYRLQQAQKGTPMKPNTQATPP

SEQ ID NO: 35 - protein, 193 aa, Artificial sequence
  synthetic sequence
DYKDDDDKGSMAQVQLVESGGSLVQPGGSLRLSCAASGRFAESSSMGWFRQAPGKEREFVAAISWSGGATNYADSAKGRFTLSRDNTKNTVYLQMNSLKPDDTAVYYCAANLGNYISSNQRLYGYWGQGTQVTVSSPFTGSIVIITVVAAAVIMGTAGLSTYLYNRQRKIKKYRLQQAQKGTPMKPNTQATPP

SEQ ID NO: 36 - protein, 199 aa, Artificial sequence
  synthetic sequence
DYKDDDDKGSKQLKQIEKRLKQIEKRLQAKEWEKAQLRKELQALRKKLAQLREMLDAPTDLQVTNVTDTSITVSWTPPSATITGYRITYTPSNGPGEPKELTVPPSSTSVTITGLTPGVEYVVSVYALKDNQESPPLVGTQTTGGGSIVIITVVAAAVIMGTAGLSTYLYNRQRKIKKYRLQQAQKGTPMKPNTQATPP

SEQ ID NO: 37 - protein, 313 aa, Artificial sequence; identical to SEQ ID NO: 13
  synthetic sequence

SEQ ID NO: 38 - protein, 424 aa, Artificial sequence; identical to SEQ ID NO: 14
  synthetic sequence

SEQ ID NO: 39 - protein, 462 aa, Artificial sequence
  synthetic sequence
YPYDVPDYAGAQPARPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKGSILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 40 - protein, 427 aa, Artificial sequence
  synthetic sequence
DYKDDDDKGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 41 - protein, 788 aa, Artificial sequence
  synthetic sequence
MTIYQFLRLFVLWACLPHFCCPELTFRRTPGIQQMTAESRAPRSDGKILHRQKRGWMWNQFFLLEEYTGSDYQYVGKLHSDQDKGDGSLKYILSGDGAGTLFIIDEKTGDIHATRRIDREEKAFYTLRAQAINRRTLRPVEPESEFVIKIHDINDNEPTFPEEIYTASVPEMSVVGTSVVQVTATDADDPSYGNSARVIYSILQGQPYFSVEPETGIIRTALPNMNRENKEQYQVVIQAKDMGGQMGGLSGTTTVNITLTDVNDNPPRFPQNTIHLRVLESSPVGTAVGSVKATDADTGKNAEVDYRIIDGDGTDMFDIITEKDTQEGIITVKKPLDYENRRLYTLKVEAENTHVDPRFYYLGPFKDTTIVKISIEDVDEPPVFSRSSYLFEVHEDIEVGTIIGTVMARDPDSTSSPIRFTLDRHTDLDRIFNIHSGNGSLYTSKPLDRELSQWHNLTVIAAEINNPKETTRVSVFVRILDVNDNAPQFAVFYDTFVCENARPGQLIQTISAVDKDDPLGGQKFFFSLAAVNPNFTVQDNEDNTARILTRKNGFNRHEISTYLLPVVISDNDYPIQSSTGTLTIRVCACDSQGNMQSCSAEALLLPAGLSTGALIAILLCIIILLVIVVLFAALKRQRKKEPLILSKEDIRDNIVSYNDEGGGEEDTQAFDIGTLRNPAAIEEKKLRRDIIPETLFIPRRTPTAPDNTDVRDFINERLKEHDLDPTAPPYDSLATYAYEGNDSVAESLSSLESGTTEGDQNYDYLREWGPRFNKLAEMYGGGESDKDA

SEQ ID NO: 42 - protein, 425 aa, Artificial sequence
  synthetic sequence
DYKDDDDKGSDIQMTQSPSSLSASVGDRVTITCRASQSVSSAVAWYQQKPGKAPKLLIYSASDLYSGVPSRFSGSRSGTDFTLTISSLQPEDFATYYCQQFSSWWGLFTFGQGTKVEIKGTTAASGSSGGSSSGAEVQLVESGGGLVQPGGSLRLSCAASGFDFGAAYMHWVRQAPGKGLEWVAGIYASGGDTDYADSVKGRFTISADTSKNTAYLQMNSLRAEDTAVYYCARWYYGALDYWGQGTLVTVSSILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 43 - protein, 788 aa, Artificial sequence; identical to SEQ ID NO: 41
  synthetic sequence

SEQ ID NO: 44 - protein, 425 aa, Artificial sequence; identical to SEQ ID NO: 42
  synthetic sequence

SEQ ID NO: 45 - protein, 324 aa, Artificial sequence
  synthetic sequence
DYKDDDDKGSDIQMTQSPSSLSASVGDRVTITCRASQSVSSAVAWYQQKPGKAPKLLIYSASDLYSGVPSRFSGSRSGTDFTLTISSLQPEDFATYYCQQFSSWWGLFTFGQGTKVEIKGTTAASGSSGGSSSGAEVQLVESGGGLVQPGGSLRLSCAASGFDFGAAYMHWVRQAPGKGLEWVAGIYASGGDTDYADSVKGRFTISADTSKNTAYLQMNSLRAEDTAVYYCARWYYGALDYWGQGTLVTVSSGSIIPIVAGVVAGIVLIGLALLLIWKLLMIIHDRREFAKFEKEKMNAKWDTGENPIYKSAVTTVVNPKYEGK

SEQ ID NO: 46 - protein, 275 aa, Artificial sequence
  synthetic sequence
DYKDDDDKGSDIQMTQSPSSLSASVGDRVTITCRASQSVSSAVAWYQQKPGKAPKLLIYSASDLYSGVPSRFSGSRSGTDFTLTISSLQPEDFATYYCQQFSSWWGLFTFGQGTKVEIKGTTAASGSSGGSSSGAEVQLVESGGGLVQPGGSLRLSCAASGFDFGAAYMHWVRQAPGKGLEWVAGIYASGGDTDYADSVKGRFTISADTSKNTAYLQMNSLRAEDTAVYYCARWYYGALDYWGQGTLVTVSSILGILGGILALLILILLLLLFRS

(19) United States
(12) Patent Application Publication
(10) Pub. No.: US 2023/0405120 A1
(43) Pub. Date: Dec. 21, 2023

(54) ENGINEERED CELLULAR ADHESION MOLECULES AND METHODS OF USE THEREOF

(71) Applicant: The Regents of the University of California, Oakland, CA (US)

(72) Inventors: Wendell A. LIM, San Francisco, CA (US); Adam J. STEVENS, San Francisco, CA (US)

(21) Appl. No.: 18/030,662

(22) PCT Filed: Nov. 1, 2021
(86) PCT No.: PCT/US2021/057601

§ 371 Date: Apr. 6, 2023

Related U.S. Application Data
(60) Provisional application No. 63/108,764, filed on Nov. 2, 2020.

(51) Int. Cl. A61K 39/00; C07K 14/47; C07K 16/18; C12N 5/077

(52) U.S. Cl. CPC A61K 39/4633; C07K 14/47; C07K 16/18; C12N 2533/50; C07K 2319/03; C07K 2317/622; C12N 2537/10; C12N 5/0656

(57) ABSTRACT

Described herein is an engineered cell adhesion molecule. The engineered cell adhesion molecule is a fusion protein comprising: an extracellular binding domain comprising a first binding moiety, a transmembrane domain and an intracellular domain that is capable of signaling to and reorganize the cytoskeleton of the cell upon specific binding of the first binding moiety to a second binding moiety. Various compositions, cells and methods that employ the cells are also described.

### CROSS-REFERENCING

This application claims the benefit of U.S. provisional application Ser. No. 63/108,764, filed on Nov. 2, 2020, which application is incorporated by reference herein.

### GOVERNMENT RIGHTS

This invention was made with government support under grant no. U54 CA244438 awarded by The National Institutes of Health. The government has certain rights in the invention.

### BACKGROUND

Cell adhesion is the process by which cells interact and attach to neighboring cells through specialized molecules of the cell surface. Cell adhesion can occur through direct cell-cell interactions, or indirectly via interactions with the surrounding extracellular matrix (ECM). Cell adhesion occurs from the interactions between cell-adhesion molecules (CAMs), which are transmembrane proteins located on the cell surface.

Cell adhesion is crucial for the assembly of individual cells into the three-dimensional tissues. In other words, cells do not simply “stick” together to form tissues, but rather are organized into very diverse and highly distinctive patterns. Cell adhesion is responsible for assembling cells together and, along with their connections to the internal cytoskeleton, determine the overall architecture of the tissue. Thus, cell adhesion is a mechanism by which basic genetic information can be translated into the complex three-dimensional patterns of cells in tissues. In addition, to keeping cells in position, cell adhesion also facilitates cell locomotion.

There are a number of diseases caused by dysfunctional cell adhesion. For example, loss of cell adhesion occurs during cancer metastasis, which allows metastatic tumor cells to escape their site of origin and spread through the circulatory system. Other CAMs, like selectins and integrins, can facilitate metastasis by mediating cell-cell interactions between migrating metastatic tumor cells in the circulatory system with endothelial cells of other distant tissues. Other human genetic diseases are caused by an inability to express specific adhesion molecules. For example, in leukocyte adhesion deficiency-I (LAD-I) expression of the β2 integrin subunit is reduced or lost, which leads to reduced expression of β2 integrin heterodimers. These proteins are required for leukocytes to firmly attach to the endothelial wall at sites of inflammation in order to fight infections. Leukocytes from LAD-I patients are unable to adhere to endothelial cells and patients exhibit serious episodes of infection that can be life-threatening. In another example, an autoimmune disease called pemphigus is also caused by loss of cell adhesion. In this disease, autoantibodies targeting a person's own desmosomal cadherins leads to epidermal cells detaching from each other and causes skin blistering.

Despite all that is known about the phenomenon, cell adhesion has not yet been harnessed in way that would allow one cell to adhere to another cell by design. This disclosure provides a way to customize cell adhesion in a programmable and interchangeable manner, thereby allowing therapeutic cells to adhere to target cells within a body or allowing one to build complex tissues from isolated cells in vitro, among other things.

### SUMMARY

Provided herein are modified cellular adhesion molecules (CAMs) that impart custom adhesive capabilities. These modified cellular adhesion molecules, which may also be referred to as orthogonal or synthetic CAMs (“OrthoCAMs” or “SynCAMs”) in this disclosure, have an altered extracellular recognition domain that provides new binding capabilities but retain the native signaling functions of an endogenous cellular adhesion molecule. As will be described in greater detail below, SynCAMs can be engineered by replacing the extracellular domain (ECD) of an endogenous cellular adhesion molecule with a new recognition domain (e.g., the antigen binding domain of an antibody) that specifically binds to another protein (e.g., an antigen) on another cell or a tissue scaffold. Cell adhesion can be customized by pairing different intracellular domains of endogenous cellular adhesion molecules with different extracellular recognition domains, allowing one to control variety of phenomena by design, e.g. the formation of adherens or tight junction, recruitment of the cytoskeleton, polarization of membrane proteins, etc. This control allows one to produce designer tissues that have a pre-defined cellular organization and composition, for example, as well as the ability to localize cellular therapies to pre-defined sites within a body.

For example, SynCAMs can be used to control the spatial organization of cells in multi-cellular tissues that are fabricated from single cells in vivo or ex vivo. In another example, SynCAMs can be used to adhere cells to each other in vitro or in vivo. The second cell may be another engineered cell or a non-recombinant cell. In these embodiments, the non-recombinant cell may express an antigen to which the SynCAMs may bind. In another example, two engineered cells may adhere with each other via a third molecule, e.g., a soluble protein. In these embodiments, the different cells may bind to different sites on the third component. In addition, cells can be bound to a matrix (e.g., a tissue scaffold) via a SynCAM. In these embodiments, the matrix may be an engineered matrix or a natural matrix to which the SynCAM has been designed to bind. The terms matrix and scaffold are intended to include natural and non-natural extracellular matrices, materials, and hydrogels, etc.

In cell-cell adhesion embodiments, the interactions can be heterotypic (where the SynCAM have extracellular binding domains that directly or indirectly bind to one another as a heterodimer, meaning that different cells can be paired with one another) or homotypic (where the SynCAM have extracellular binding domains that directly bind to one another as a homodimer, meaning that single cells of the same type can be clumped together, if desired). The type of interaction can be tuned by selecting an appropriate extracellular domain (e.g., which domain controls the identity and/or strength of the extracellular interaction) and/or by selecting an appropriate intracellular domain (i.e., which domain controls the strength, lifetime, and/or mechanical properties adherence). For example, some SynCAM may be used to produce custom polarized cell-cell synapses (akin to neuronal synapses, immune synapse, tight junctions). In some embodiments, different layers of control can be combined within a single cell or in a population of cells. For example, engineered cells can be programmed to assemble with other engineered cells (or with other engineered cells and non-recombinant cells) in a pre-defined way.

A method for altering the binding characteristics of a cell is provided. In some embodiments, this method comprises introducing a nucleic acid encoding a fusion protein as described above into the cell, wherein the introducing results in expression of the fusion protein and alteration of the binding characteristics of the cell.

In some cases, the extracellular binding domain binds to a tissue-specific surface molecule and expression of the fusion protein in the cell results in a longer residency in a selected tissue relative to the same cell without the fusion protein. In these embodiments, the residency of therapeutic cells (i.e., longer residence time and/or slower egress) in or on a tissue in a body can be increased. In these embodiments, a synthetic adhesion molecule that recognizes a tissue-specific surface molecule can be used to localize engineered therapeutic cells to a specific organ (e.g., brain, kidney, or gut, etc) where they can carry out a therapeutic function.

In some cases, the extracellular binding domain binds to a disease-specific surface molecule and expression of the fusion protein in the cell results in a longer residency in a diseased tissue relative to the same cell without the fusion protein. In these embodiments, the residency of therapeutic cells (i.e., longer residence time and/or slower egress) in or on a tissue that contains diseased cells can be increased. In these embodiments, a synthetic adhesion molecule that recognizes disease-specific surface molecule (e.g., in a tumor, or site of autoimmunity/inflammation, tissue degeneration, etc.) can be used to localize engineered therapeutic cells to a diseased area where they might carry out therapeutic function

In some cases, the extracellular binding domain binds to a molecule on the surface of a target cell and i. increases the formation of multicellular tissues with a defined structure in vitro or in vivo, controls cell sorting based on differential adhesion strengths; ii. controls autonomous sorting of cells based on differential adhesion strengths; iii. directs the assembly of an organoid in a disease model; iv. directs the assembly of an organ or tissue; v. directs regeneration of a tissue or organ in vivo; vi. assists in the formation of epithelial-like cell assemblies; or vii. directs specific cell-cell connectivities, including multicell circuit/communication systems. These uses are described in greater detail below.

In some cases, binding enhances, inhibits or modulates the function of another cell-cell interaction molecules (e.g., CARs, synNotch, TCR, FcR, Notch, growth receptors, etc.) and use in engineering multi-antigen target AND or NOT gates. In other cases, the cell may abrogate dysfunctional adhesion; or directs or enhances phagocytosis of cognate target cells. For example, binding may direct or enhance phagocytosis of cognate targets (e.g., to clear disease cells, enhance antigen presentation and spreading; potentially in macrophages, dendritic cells, microgllia, kupfler cells, etc.).

SynCAM cells can be used in a variety of applications. For example, SynCAM cells can be used to make custom tissues e.g., synthetic or semi-synthetic tissues that have a pre-determined and customizable spatial organization of cells in vitro, in vivo or ex vivo. Such tissues can be used for a variety of purposes, e.g., tissue regeneration, wound healing, fibrosis treatment, or transplants, etc. In another example, therapeutic cells can be engineered to bind to and reside in specific tissues in an antigen-specific way. For example, therapeutic immune cells could be targeted to reside in a particular tissue or tumor. Such cells could be used to target cancer, autoimmunity, or fibrosis, for example.

Because nature has evolved endogenous adhesion molecules to regulate an array of intracellular signaling functionalities that are critical to multicellular organisms, harnessing such a capability in a programmable and interchangeable manner (i.e., imparting control of targeting, strength, and type of signaling response) should prove a valuable asset to synthetic cellular therapeutics.

These and other advantages may be become apparent in view of the following discussion.

### BRIEF DESCRIPTION OF THE FIGURES

The skilled artisan will understand that the drawings, described below, are for illustration purposes only. The drawings are not intended to limit the scope of the present teachings in any way.

FIG. 1 schematically illustrates an engineered cell adhesion molecule.

FIG. 2 schematically illustrates an interaction between an engineered cell adhesion molecule on one cell and a cell adhesion molecule on another cell.

FIGS. 3A-3C schematically illustrates some uses of the engineered cell adhesion molecule.

FIG. 4 schematically illustrates some of the design principles of the SynCAMs used in the experimental section of this disclosure.

FIG. 5 schematically illustrates the SynCAMs used in Example 1, the data for which is shown in FIGS. 7A-7D.

FIGS. 6A-6D show the sequences of the SynCAMs used in Example 1, the data for which is shown in FIGS. 7A-7D. Epitope tags are shown in italics, the transmembrane and intracellular domains are underlined in bold and the extracellular domains are inbetween epitope tags and the transmembrane domains. Each plasmid also contains a cleavable signal sequence for trafficking to the cell membrane that is not shown in the final protein product.

FIGS. 7A-7D Synthetic adhesion molecules shape cell-cell adhesion interfaces in L929 mouse fibroblast cells. A. Cartoon depicting a cell-cell interface between one cell expressing a synCAM with a llama anti-GFP (LaG) nanobody extracellular recognition domain fused to a cellular adhesion molecule (CAM) transmembrane and intracellular domain and another cell expressing a GFP extracellular domain and CAM transmembrane and intracellular domain. B. Representative fluorescent confocal microscopy images of a cell-cell interface between a cell expressing a GFP-synCAM and cytosolic BFP bound to an adjacent cell expressing a LaG synCAM with cytosolic mCherry. The BFP and Mcherry channels are shown on top with the GFP channel shown below. Each cell pair corresponds to the indicated transmembrane and intracellular signaling domain, with “tether” corresponding to an E-Cadherin transmembrane domain and no intracellular signaling domain. C. Box and whiskers plot of the contact angle between L929 cells at a cell-cell interface between cells expressing a GFP-LaG pair with the indicated transmembrane and signaling domain (n>10). D. Box and whiskers plot of relative GFP enrichment at the cell-cell interface of L929 cells expressing the indicated synthetic adhesion molecule transmembrane and signaling domains.

FIG. 8 schematically illustrates the SynCAMs used in Example 2, the data for which is shown in FIG. 10.

FIG. 9 shows the sequences of the SynCAMs used in Example 2, the data for which is shown in FIG. 10. Epitope tags are shown in italics, the transmembrane and intracellular domains are underlined in bold and the extracellular domains are inbetween epitope tags and the transmembrane domains. Each plasmid also contains a cleavable signal sequence for trafficking to the cell membrane that is not shown in the final protein product

FIG. 10 Varying extracellular domain affinity contributes to synCAM adhesion strength. Top: Cartoon depicted the difference in affinity between the LaG16 and LaG17 nanobodies in their interaction with GFP (0.7 nM and 50 nM respectively). Bottom: Maximum projection of fluorescent microscopy images from a competition assay of L929 mouse fibroblast cell adhesion. Cells expressing LaG16-Ecad and cytosolic mCherry (orange) were mixed with cells expressing GFP-Ecad (green) and cells expressing LaG17-Ecad and cytosolic blue fluorescent protein (blue).

FIG. 11 schematically illustrates the SynCAMs used in Example 3, the data for which is shown in FIG. 13.

FIGS. 12A-12C show the sequences of the SynCAMs used in Example 3, the data for which is shown in FIGS. 13A-13D. Epitope tags are shown in italics, the transmembrane and intracellular domains are underlined in bold and the extracellular domains are inbetween epitope tags and the transmembrane domains. Each plasmid also contains a cleavable signal sequence for trafficking to the cell membrane that is not shown in the final protein product

FIGS. 13A-13D Programmability of SynCAM extracellular domain. A. Cartoon depicting the programmability of SynCAM heterophilic extracellular domains, with the color of each extracellular domain (red, orange, pink, green) representing a unique binding pair. B. Fluorescent confocal images (t=1 hr) of L929 cell assemblies expressing synthetic adhesion molecule pairs containing an ICAM transmembrane and intracellular domain and the indicated protein interaction domain (HA/aHA, EGFR/aEGFR, MBP/aMBP, c-Met/aC-Met, CD19/aCD19, Mcherry/LaM). C. Cartoon depicting two strategies to program synCAM homophilic assemblies. Top: coexpression of both members of a heterophilic synCAM pair in the same cell. Bottom: Expression of a synCAM containing a homophilic leucine zipper extracellular domain. D. Fluorescent confocal images (t=24 hr) of homophilic L929 cell assemblies expressing a synCAM containing the indicated extracellular recognition domain and an ICAM transmembrane and intracellular signaling domains. The MBP/aMBP and HA/aHA pairs are examples of coexpression of heterophilic adhesion molecules, and the Aph4 assembly is a synCAM containing the Aph4 extracellular leucine zipper, which binds homophilically.

FIGS. 14A-14B schematically illustrate the SynCAM used in Example 4, the data for which is shown in FIGS. 16A-16B.

FIGS. 15A-15B show the sequences of the SynCAMs used in Example 4, the data for which is shown in FIGS. 16A-16B. Epitope tags are shown in italics, the transmembrane and intracellular domains are underlined in bold and the extracellular domains are inbetween epitope tags and the transmembrane domains. Each plasmid also contains a cleavable signal sequence for trafficking to the cell membrane that is not shown in the final protein product.

FIGS. 16A-16B: SynCAM mediated complex pattern formation. A. Fluorescent confocal image of cell assembly of mouse fibroblast L929 cells with Cell A expressing a CD19-Ecad synCAM and cytosolic BFP (blue) Cell B expressing aCD19-Ecad synCAM, LaG16-Ecad, and cytosolic mCherry (orange), and Cell C expressing GFP-Ecad synCAM (green) (t=3 hr). B. Fluorescent confocal image of semisynthetic cellular assembly of mouse fibroblast L929 cells with Cell A expressing CDH10 (green) and Cell B expressing aCDH10-Ecad synCAM (orange) (t=24 hr).

FIG. 17 schematically illustrates the SynCAMs used in Example 5, the data for which is shown in FIGS. 19A-19B.

FIG. 18 shows the sequences of the SynCAMs used in Example 5, the data for which is shown in FIGS. 19A-19B. Epitope tags are shown in italics, the transmembrane and intracellular domains are underlined in bold and the extracellular domains are inbetween epitope tags and the transmembrane domains. Each plasmid also contains a cleavable signal sequence for trafficking to the cell membrane that is not shown in the final protein product.

FIGS. 19A-19B: SynCAMs control adhesion in primary T cells. A. Cartoon depicting adhesion between L929 mouse fibroblast cells expressing CDH10 and primary human T cells expressing aCDH10 synCAMs. B. 3D reconstruction of a confocal image from a mixture of L929 mouse fibroblast cells expressing CDH10 (green) and primary human T cells expressing the indicated aCDH10 synCAM (CDH10-Ecad or CDH10-Beta1 integrin, orange), a control lacking an intracellular signaling domain (aCDH10 AICCD, blue) or a control only expressing cytosolic mCherry (orange) (t=24 hr).

FIGS. 20A-20B: Design of synthetic cellular adhesion molecules. (a) Physiological roles of cellular adhesion in mediating tissue organization (left), cell trafficking (center) and synaptic formation (right). (b) Conceptual design of synCAM receptors. The extracellular domain of a CAM (left) is replaced by GFP and a GFP− binding nanobody (αGFP, right). A “tether” control lacking an ICD is also shown (middle).

FIGS. 21A-21D: Synthetic Adhesion Molecules (synCAMs) facilitate custom cell-cell interaction properties. (a) Top: Maximum projection of 20× confocal microscopy images of pairwise synCAM interfaces (t=3 hr): GFP-expressing cell (blue) is bound to an aGFP expressing cell (orange). The CAM TM and ICD domain for each pair is indicated (tether=control lacking ICD, Dll1=Delta-like Protein 1, JAM-B=Junction Adhesion Molecule B, NCAM-1=Neural Cell Adhesion molecule 1, MUC-4=Mucin 4, ICAM-1=Intercellular Adhesion Molecule 1, Ecad=E-cadherin, Intβ1=beta 1 integrin, Intβ2=beta 2 integrin). Bottom: GFP channel of the interfaces above highlighting differences of receptor enrichment at the interface. (b) Box and whisker plots of contact angles measured from the interfaces shown in a (n=15-20 pairs). In addition, contact angles for wild type Ecad (WT Ecad) homotypic cell-cell interaction are shown. (c) Box and whisker plots of fraction GFP enrichment at the cell-cell interface from a are shown (n=15-20 pairs). (d) Depiction of known recruitment interactions of downstream signaling proteins found in cell adhesion molecule ICDs.

FIGS. 22A-22C: SynCAM intracellular domains yield distinct mechanical and cytoskeletal properties. (a-c) Representative phalloidin-stained images of L929 cells expressing the indicated synCAMs spreading on a GFP-coated surface (t=2 hr). Actin (phalloidin stain) is shown in white; full footprint of cell (membrane label) is outlined in blue. All images are shown at same scale. (a) L929 cell expressing anti-GFP tether (no ICD) shows minimal spreading). (b) L929 cells expressing synCAMs with ICDs from Ecad, ICAM, Integrin b1, Integrin b2 show contractile spreading phenotype—cell spreads in circular manner with cortical actin at the periphery of the cell footprint. (c) L929 cells expressing synCAMs with ICDs from NCAM, JAM-B, MUC-1, and DLL1 show protrusive spreading phenotype (a.k.a “fried egg” shape)—cortical actin does not spread very far, but cell membrane footprint extends in very thin layer beyond in bulk of cell, often with less circularity (i.e. more filopodial or lamellopodial nature).

FIGS. 23A-23D: Tuning synthetic adhesion binding strength through varying ECD affinity or intracellular signaling. (a) Quantification of contact angles from pairwise L929 cells expressing GFP/aGFP synCAMs with the indicated affinities and presence (blue) or absence (black) of an ICAM-1 signaling domain (n=20 cells, t=3 hr). (b) Depiction of the cell sorting competition assay. L929 cells expressing GFP-ICAM-1 (green) are mixed with L929 cells expressing one of two different ICAM-1 synCAMs (orange or blue cells). The energetically favorable binding interaction sorts to the center of the sphere. Values correspond to difference in average distance to the center of the sphere (BFP—mCherry), with larger values indicating a preference for mCherry binding. (c) Quantification of cell sorting competition assay between 1929 cells expressing aGFP-ICAM-1 with the indicated ECD affinity (mCherry or BFP) mixed with L929 cells expressing GFP-ICAM-1 (t=24 hr). (d) Quantification of cell sorting competition assay in which L929 cells expressing an aGFP synCAM (orange) or tether (blue) with the indicated ECD affinity for GFP are mixed with L929 cells expressing GFP-ICAM-1 (t=24 hr).

FIGS. 24A-24C: Design of synCAMs with orthogonal extracellular interaction specificity. (a) Left: Schematic of heterophilic synCAMs with orthogonal extracellular recognition domains. Right: Maximum projection of 20× confocal microscopy cell-cell interface images. L929 cells expressing synCAMs with the indicated antibody-antigen pair ECDs and either ICAM (top) or beta 1 integrin (bottom) TM/ICDs (t=3 hr) are shown. (b) Left: synCAM design with a homophilic binding leucine zipper ECD. Right: Maximum projection of 20× confocal microscopy images of L929 cells expressing homophilic binding synCAMs with the Aph4 or IF1 leucine zippers ECD and ICAM-1 TM/ICDs (ULA round bottom well, 80 cells total, t=24 hr). (c) Left: cartoon depicting the receptor design and differential sorting assay of L929 cells expressing WT P-cadherin (WT Pcad, orange) and an aPcad synCAM (aPcad, blue). The aPcad synCAM contains an ICAM-1 TM/ICD. Right: maximum projection images of the sorting assay in which L929 cells expressing WT Pcad (orange) are mixed with parental (top) or synCAM (bottom) 1929 cells (blue, t=0, 24 hr).

FIGS. 25A-25C: Engineering custom multicellular assemblies. (a) Maximum projection of 20× confocal microscopy images of L929 cells expressing synCAMs with the indicated ECD recognition partners (t=2 hr). Assemblies with alternating “A-B” (left), bridging “A-B-C” (middle), and cyclic “A-B-C” (right) patterning are all shown. (b) Example images of isolated cyclic interactions (t=2 hr) generated from the L929 cells utilized in a (right panel). (c) 20× confocal microscopy images of differential sorting between L929 cells expressing WT Ecad or the indicated homophilic-binding synCAMs (t=24 hr).

FIGS. 26A-26B: Using synCAMs to modify native cell interactions. (a) Modulation of L929 cell sorting mediated by WT Ecad (blue) and WT Pcad (orange) through coexpression of heterophilic synCAMs or tether receptors. Maximum projections of 20× confocal microscopy images are shown (t=24 hr). (b) Maximum projection images of 1929 cells expressing WT Pcad (orange) mixed with an MDCK monolayer (blue). A GFP/aGFP interaction (tether or ICAM-1 synCAM) is introduced to alter the topographies of the two layers (t=24 hr).

### DEFINITIONS

Unless otherwise defined, all technical and scientific terms used herein have the same meaning as commonly understood by one of ordinary skill in the art to which this invention belongs. Still, certain elements are defined for the sake of clarity and ease of reference.

Terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Operably linked” refers to a juxtaposition wherein the components so described are in a relationship permitting them to function in their intended manner. For instance, a promoter is operably linked to a coding sequence if the promoter affects its transcription or expression.

A “vector” or “expression vector” is a replicon, such as plasmid, phage, virus, or cosmid, to which another DNA segment, i.e. an “insert”, may be attached so as to bring about the replication of the attached segment in a cell.

“Heterologous,” as used herein, means a nucleotide or polypeptide sequence that is not found in the native (e.g., naturally-occurring) nucleic acid or protein, respectively.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. The antibodies can also be bound to a solid support, including, but not limited to, polystyrene plates or beads, and the like. Also encompassed by the term are Fab′, Fv, F(ab′)2, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “nanobody” (Nb), as used herein, refers to the smallest antigen binding fragment or single variable domain (VHH) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a VHH antibody.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

“Single-chain Fv” or “sFv” or “scFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “affinity” refers to the equilibrium constant for the reversible binding of two agents (e.g., an antibody and an antigen) and is expressed as a dissociation constant (KD). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. In some cases, the first member of a specific binding pair present in the extracellular domain binds specifically to a second member of the specific binding pair. “Specific binding” refers to binding with an affinity of at least about 10-7 M or greater, e.g., 5×10-7 M, 10-8 M, 5×10-8 M, and greater. “Non-specific binding” refers to binding with an affinity of less than about 10-7 M, e.g., binding with an affinity of 10-6 M, 10-5 M, 10-4 M, etc.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) Sadelain et al. Cancer Discov (2013) 3(4):388-98; Cartellieri et al., J Biomed Biotechnol (2010) 956304; the disclosures of which are incorporated herein by reference in their entirety.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

Other definitions of terms may appear throughout the specification. It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### DETAILED DESCRIPTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

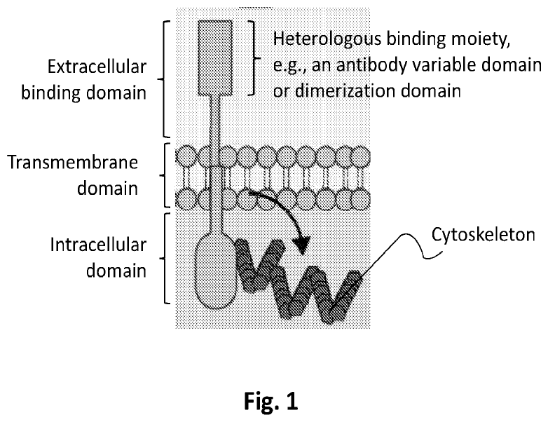

Provided herein is a composition an engineered cell adhesion molecule (a “fusion protein”), a nucleic acid encoding the same and an engineered cell comprising the same. The engineered cell adhesion molecule comprises three domains: an extracellular binding domain, one or more transmembrane domains and an intracellular domain, where the wherein the extracellular binding domain and the intracellular binding domain of the fusion protein are “heterologous” i.e., not from the same native cell adhesion molecule. As shown in FIG. 1, the extracellular binding domain comprises a first binding moiety that is heterologous to the protein and capable of specifically binding to a second binding moiety. For example, the first binding moiety could be the antigen-binding domain of an antibody or a dimerization domain. More examples of antigen-binding domains are provided below. The extracellular binding domain may or may not bind to the extracellular domain of a naturally-occurring cell adhesion molecule (such as the extracellular domain of a naturally-occurring adherin or integrin, etc.) or the extracellular matrix as found in multicellular organisms (e.g., mammals). In some embodiments, the first binding moiety does not bind to the extracellular domain of a naturally-occurring cell adhesion molecule.

In any embodiment, the first binding moiety is capable of specifically binding: (a) to a naturally-occurring protein expressed on the surface of a partner cell; (b) to a non-naturally-occurring protein expressed on the surface of a partner cell; (c) to a scaffold molecule or material bearing the cognate ligand; (d) to a partner cell via a homophilic interaction; (e) to partner cells via a heterophilic interaction; (f) to multiple partner cells or substrates via a multivalent interaction; (g) to a partner cell or substrate via a chemically inducible interaction; (h) to a partner cell or substrate via light- or protease-activated interaction; and/or (i) to multiple partner cells or substrates via tandem recognition domain.

The intracellular domain of the engineered cell adhesion molecule, in the other hand, may be the intracellular domain of a naturally-occurring cell adhesion molecule or a variant thereof that retains its ability to engage with and apply local control over the cytoskeleton, e.g., to reinforce the linkage and/or locally control cytoskeletal filament polymerization. Thus, the intracellular domain of the cell adhesion molecule is capable of signaling to and reorganizing the cytoskeleton of the cell upon specific binding of the first binding moiety of the cell adhesion molecule to a second binding moiety. Because signaling of many cell adhesion molecules is triggered by physical force (e.g., a force that “tugs” at the molecule), the second binding moiety that activates signaling should be at least partially immobilized or tethered, i.e., not in solution. For example, the second binding moiety that triggers signaling may be on the surface of another cell or tissue scaffold, or tethered to another cell or tissue scaffold, for example.

As shown in FIG. 1, the engineered cell adhesion molecule also contains one or more transmembrane domains (which should be in between the extracellular and intracellular domains). In some embodiments, e.g., the engineered cell adhesion molecule may have a single transmembrane domain. In other embodiments, the engineered cell adhesion molecule may have multiple transmembrane domains. The transmembrane domain of an engineered cell adhesion molecule can be the transmembrane domain of a naturally-occurring cell adhesion molecule (e.g., the same naturally-occurring cell adhesion molecule as the intracellular domain). However, this is not necessary because the transmembrane domain can be readily designed using hydrophobic amino acids or a transmembrane domain from another transmembrane protein can be used. As would be apparent, the fusion protein may have other sequences, e.g., linkers, effector domains, signaling domains, etc., in addition to the domains that are specifically described herein.

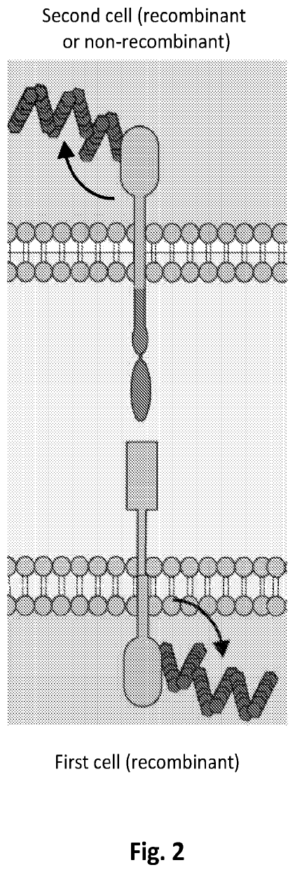

In some embodiments, the composition may further comprise a second cell, where the first cell (i.e., the recombinant cell, as described above) and the second cell adhere to each other via an interaction that is initiated by binding of the first binding moiety of the engineered cell adhesion molecule to a second binding moiety on the second cell. The second cell may be different type of cell to the recombinant cell. For example, the recombinant cell may be an immune cell whereas the second cell may be a cancer cell, or the recombinant cell may be from a particular tissue type (e.g., muscle) and the second cell may be from a different tissue. In some embodiments, the recombinant cell and the second cell may adhere to each other directly via binding of the first binding moiety to a second binding moiety that is on the surface of second cell. In these embodiments, the second cell may be non-recombinant or recombinant. In embodiments in which the second cell is non-recombinant, the ligand may be an antigen on the surface of second cell, e.g., a tissue-specific or disease-specific antigen. In embodiments in which the second cell is recombinant, the second cell may also have an engineered cell adhesion protein on its surface. For example, if the second cell is recombinant, then it may comprise on its surface a second engineered cell adhesion molecule comprising: a second extracellular binding domain comprising a second binding moiety (i.e., to which the first binding moiety of the other cell binds), where the second extracellular binding domain does not contain the extracellular binding domain of a native cell adhesion molecule, a transmembrane domain, and an intracellular domain that is capable of signaling to the cytoskeleton of the cell upon binding of the first binding moiety to the second binding moiety. In these embodiments, the recombinant cell and the second cell adhere to each other in a process that is initiated by binding of the first binding moiety to the second binding moiety. An example of this embodiment is illustrated in FIG. 2.

In addition to embodiments in which the recombinant cell and the second cell may adhere to each other directly, the recombinant cell and the second (recombinant) cell can also adhere to each other directly via a soluble bridging molecule, e.g., a soluble protein, to which both engineered cell adhesion molecules bind. In these embodiments, the engineered cell adhesion molecules on the different cells may bind to different sites (e.g., epitopes) on the soluble bridging molecule.

In any embodiment in which cells adhere to another, the interaction between the cells may be homotypic (in which case the first binding moiety homodimerizes, i.e., binds to itself) or heterotypic (in which case the first and second binding moieties are different and they heterodimerize, i.e., binds to one another). These interactions may be in vivo (within the body of a mammal), in vitro (using cultured cells) or ex vivo (using cells that have been isolated from a mammal).

In some embodiments, the interactions between the first and second binding moieties may be conditional, i.e., inducible. For example, binding between the first binding moiety to the first binding moiety by be light or chemically inducible.

In some embodiments, the composition may further comprise a tissue scaffold. In these embodiments the recombinant cell adheres to the tissue scaffold via binding of the first binding moiety to the scaffold. In these embodiments, the scaffold may be a non-naturally occurring scaffold or it may be a naturally occurring scaffold that has been coated (directly or indirectly) with the second binding moiety.

The identity of the first binding moiety of the engineered cell adhesion molecule may vary greatly, since all that moiety needs to do is specifically bind to its binding partner, the second binding moiety. Pairs of binding partner that bind to one another are numerous and include, without limitation: an antibody variable domain (e.g., scFvs, and nanobodies) and sequence to which the antibody variable domain binds (which are almost limitless), a T cell receptor variable domains and the sequence to it binds, TCRα, TCRβ, a receptor and a peptide to which that receptor binds, a ligand for a cell adhesion protein, a pair of leucine zippers, two halves of a split intein, etc. PDZ proteins, proteinase inhibitors, etc., can also be used. In embodiments that use TCRσc, or TCRβ, one may need both TCRα and TCRβ coexpressed, but one could fuse them to the same intracellular adhesion signaling domain or two separate signaling domains. For example, one could express both a TCR-alpha-Beta-integrin chimera and a TCR beta-JAM chimera. This would allow you to recognize MHC-peptide extracellular and then respond with either a single or double adhesion signaling-based response.

As noted, above, in some cases binding of the first and second binding moieties may be conditional. In these embodiments, binding the first and second binding moieties may be only in the presence of dimerization agent. Examples of pairs of protein domains that conditionally dimerize with one another include: FKBP and FKBP (which dimerize in the presence of rapamycin), FKBP and CnA (which dimerize in the presence of rapamycin), FKBP and cyclophilin (which dimerize in the presence of rapamycin), FKBP and FRG (which dimerize in the presence of rapamycin), GyrB and GyrB (which dimerize in the presence of coumermycin), DHFR and DHFR (which dimerize in the presence of methotrexate), DmrB and DmrB (which dimerize in the presence of AP20187), PYL and ABI (which dimerize in the presence of abscisic acid), Cry2 and CIB1 (which dimerize in the presence of blue light); GAI and GID1 (which dimerize in the presence of gibberellin) and a ligand-binding domain of a nuclear hormone receptor, and a co-regulator of the nuclear hormone receptor (which dimerize in the presence of a nuclear hormone, agonists thereof and antagonists thereof, e.g., tamoxifen). In embodiments in which rapamycin can serve a dimerizer, a rapamycin derivative or analog can also be used. In other embodiments, the first binding moiety may be a Snaptag/Halo tag domain.

If the first binding moiety of the engineered cell adhesion molecule is an antibody variable domain (e.g., an scFv or nanobody), then it may recognize a disease-specific or tissue-specific antigen. For example, if the engineered cell adhesion molecule recognizes a disease specific antigen, then the antigen may be a cancer-associated antigen, where cancer-associated antigens include, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-AL IL-13R-a2, GD2, and the like. Cancer-associated antigens also include, e.g., 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-0, TRAIL-R1, TRAIL-R2, tumor antigen CTAA16.88, VEGF-A, VEGFR-1, VEGFR2, and vimentin. The antigen can be associated with an inflammatory disease. Non-limiting examples of antigens associated with inflammatory disease include, e.g., AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin a4, integrin a4r37, LFA-1 (CD11a), myostatin, OX-40, scleroscin, SOST, TGF beta 1, TNF-α, and VEGF-A. Antigens for other diseases may be targeted in the same way. See, e.g., Dannenfelser (Cell Syst. 2020 11: 215-228), WO2017/193059, WO2020/097395 and PCT/US2021/045796).

The binding domain of the fusion protein may be specific for Mesothelin, FAP, Her2, Trop2, GPC3, MUC1, ROR1, EPCAM, ALPPL2, PSMA, PSCA, EG1-Rviii, EGFR, Claudin18.2, or GD2, for example. In some embodiments, a binding domain of the fusion protein may have HC and LC CDR1, 2 and 3 sequences that are identical to or similar (i.e., may contain up to 5 amino acid substitutions, e.g., up to 1, up to 2, up to 3, up to 4 or up to 5 amino acid substitutions, collectively) to the CDRs of any of the antibodies listed in the publication cited in the table below, which publications are incorporated by reference for those sequences. The framework sequence could be humanized, for example. In some embodiments, the binding domain of the fusion protein may have HC and LC variable regions that are at least 90%, at least 95%, at least 98% or at least 99% identical to a pair of HC and LC sequences listed in the publication cited in the table below, which publications are incorporated by reference for those sequences.

| Antigen | |
|---|---|
| binding | |
| domain | Exemplary sources of antigen binding sequences |
| Meso- | US 2021/0290676, US 2021/0284728 A1, US |
| thelin | 2021/0275584 A1, Feng et al., Mol. Cancer Ther. |
| (MSLN) | 8(5): 1113-1118 (2009), US 2021/0269537 A1, |
| | US 2021/0252122 A1, US 2021/0230242 A1, |
| | US 2021/0155702 A1, US 2021/0137977 |
| | A1, US 2021/01016620 A1 |
| FAP | US 2021/0252122 A1, Kakarla et al. Mol Ther. 2013 |
| | August; 21(8): 1611-20, Wang et al. Cancer Immunol |
| | Res. 2015 July; 3(7): 815-826, Petrausch et al. BMC |
| | Cancer. 2012; 12: 615, Tran et al. J Exp Med. 2013 |
| | Jun. 3; 210(6): 1125-35. |
| Her2 | US 2021/0299269, US 2021/0290676, US 2021/ |
| | 0137977 A1, US 2021/01016620 A1, US 2021/ |
| | 0299172 A1 |
| Trop2 | US 2021/0290676, Zhao et al. Am J Cancer Res. |
| | 2019; 9(8): 1846-1856., Bedoya et al. Cytotherapy |
| | 2019 May; 21(5): S11-12., Sayama et al. Mol |
| | Med Rep. 2021 February; 23(2): 92. |
| GPC3 | US 2021/0261646 A1, US 2021/0137977 A1, US |
| | 2021/01016620 A1, Li et al. Am J Transl Res. |
| | 2021 Jan. 15; 13(1): 156-167., Batra et al. Cancer |
| | Immunol Res. 2020 Mar; 8(3): 309-320. |
| MUC1 | US 2021/0269547 A1, US 2021/0155702 A1, |
| | Supimon et al. Sci Rep. 2021 Mar. 18; 11(1): 6276., |
| | Zhou et al. Front Immunol. 2019 May 24; 10: 1149., |
| | Mei et al. Cancer Med. 2020 January; 9(2): 640-652. |
| ROR1 | US 2021/0290676, Wallstabe et al JCI Insight. 2019 |
| | September 19; 4(18): e126345, US 2021/0137977 |
| | A1, Prussak et al. J. Clin. Oncol. 2020; 38, no. 6_ |
| | suppl, Srivastava et al. Cancer Cell. |
| | 2021 Feb. 8; 39(2): 193-208.e10. |
| EPCAM | US 2021/0290676, US 2021/0284728 A1, US 2021/ |
| | 0269547 A1, Qin et al. Oncoimmunology. 2020 |
| | Aug. 15; 9(1): 1806009., Deng et al. BMC |
| | Immunol. 2015 Jan. 31; 16(1): 1. |
| ALPPL2 | Su et al Cancer Res. 2020 Oct 15; 80(20): 4552-4564., |
| | Hyrenius-Wittsten et al. Sci Transl Med. 2021 Apr. |
| | 28; 13(591): eabd8836., WO2017095823A1 |
| PSMA | US 2021/0290676, US 2021/0284728 A1, US 2021/ |
| | 0269547 A1, US 2021/0252122 A1, US 2021/ |
| | 0137977 A1, US 2021/0113615 A1 |
| PSCA | US 2021/0290676, US 2021/0269547 A1, Wu et al. |
| | Biomark Res. 2020 Jan. 28; 8: 3., Dorff et al. J. Clin. |
| | Oncol. 2020; 38, no. 6_suppl, US 2020/0308300 |
| EGFRviii | US 2021/0290676, US 2021/0252122 A1, US 2021/ |
| | 0137977 A1, O'Rourke et al. Sci Transl Med. |
| | (2017) 9: eaaa0984, Abbott et al. Clin Transl |
| | Immunology. 2021 May 9; 10(5): e1283. |
| EGFR | US 2021/0290676, US 2021/0269547 A1, US 2021/ |
| | 0155702 A1, Xia et al. Clin Transl Immunology. |
| | 2020 May 3; 9(5): e01135., Li et al. Cell Death Dis. |
| | 2018 February; 9(2): 177., Liu et al. Clinical Trial |
| | Cytotherapy. 2020 October; 22(10): 573-580. |
| Claudin | US 2021/0252122 A1, Jiang et al. J Natl Cancer |
| 18.2 | Inst. 2019 Apr. 1; 111(4): 409-418., Chin et al. |
| | J Cancer Res. 2020 Apr; 32(2): 263-270., Zhan |
| | et al. J. Clin. Oncol. 2019, 37, 2509., |
| | Singh et al. J Hematol Oncol. 2017; 10: 105. |
| GD2 | US 2021/0290676, Seitz et al. Oncoimmunology. |
| | 2020; 9(1): 1683345., Chulanetra et al. Am J Cancer |
| | Res. 2020; 10(2): 674-687., Sujjitjoon et al. Transl |
| | Oncol. 2021 February; 14(2): 100971, Andersch |
| | et al. BMC Cancer. 2019 Sep. 9; 19(1): 895. |

New antigen binding domains may also be generated in the form of immunoglobulin single variable (ISV) domains. The ISV domains may be generated using any suitable method. Suitable methods for the generation and screening of ISVs include without limitation, immunization of dromedaries, immunization of camels, immunization of alpacas, immunization of sharks, yeast surface display, etc. Yeast surface display has been successfully used to generate specific ISVs as shown in McMahon et al. (2018) Nature Structural Molecular Biology 25(3): 289-296 which is specifically incorporated herein by reference.

Immunoglobulin sequences, such as antibodies and antigen binding fragments derived there from (e g, immunoglobulin single variable domains or ISVs) are used to specifically target the respective antigens disclosed herein. The generation of immunoglobulin single variable domains such as e.g., VHHs or ISV may involve selection from phage display or yeast display, for example ISV can be selected by utilizing surface display platforms where the cell or phage surface display a synthetic library of ISV, in the presence of tagged antigen. A fluorescent secondary antibody directed to the tagged antigen is added to the solution thereby labeling cells bound to antigen. Cells are then sorted using any cell sorting platform of interest e.g., magnetic-activated cell sorting (MACS) or fluorescence-activated cell sorting (FACS). Sorted clones are amplified, resulting in an enriched library of clones expressing ISV that bind antigen. The enriched library is then re-screened with antigen to further enrich for surface displayed antigen binding ISV. These clones can then be sequenced to identify the sequences of the ISV of interest and further transferred to other heterologous systems for large scale protein production.

In any embodiment, the extracellular domain of the engineered cell adhesion molecule may or may not have the amino acid sequence that is at least 80%, at least 90%, or at least 95% identical to the sequence of an extracellular domain of a wild-type cell adhesion molecule. Specifically, the extracellular domain of the engineered cell adhesion molecule may or may or not have a sequence of at least 200 amino acids, at least 100 amino acids, at least 50 amino acids, or at least 20 amino acids, that is at least 80%, at least 90%, or at least 95% identical to the intracellular domain of a cadherin, an integrin beta chain (e.g., CDH1, CDH2, CDH3, CDH4, CDH5, CDH6, CDH7, CDH8, CDH9, CDH10, CDH11, CDH12, CDH13, CDH14, CDH15, CDH16, CDH17, CDH18, CDH19, CDH2O, CDH21, CDH22, CDH23, CDH24, CDH25, CDH26, CDH27, CDH28, or CDH29, for example), an integrin beta chain (e.g., ITGB1, ITGB2, ITGB3, ITGB4, ITGB5, ITGB6, ITGB7 or ITGB8, for example), an integrin alpha chain (e.g., ITGA1, ITGA2, ITGA3, ITGA4, ITGA5, ITGA6, ITGA7, ITGA8, ITGA9, ITGA10, ITGA11, for example), a junction adhesion molecule (e.g., JAMA, JAMB or JAMC, for example), a protocadherin (e.g., e.g. pcdhal, pcdhbl, pcdhgl, for example), an immunoglobulin adhesion molecule (e.g., e.g. iCAM, MAdCAM-1, NCAM, or CD2, for example), CD44, a claudin (CDLN1, CDLN2, CDLN3, CDLN4, CDLN5, CDLN6, CDLN7, CDLN8, CDLN9, CDLN10, CDLN11, CDLN12, CDLN13, CDLN14, CDLN15, CDLN16, CDLN17, CDLN18, CDLN19, CDLN20, CDLN21, CDLN22, CDLN23, CDLN24 or CDLN25, for example), neurexin/neuroligin/nectin (e.g., (e.g. Neurexinl, Nectinl or Neuroliginl, for example), Eph or ephrin (e.g. EPHA, EPHB, EFNA or EFNB, for example), a notch ligand (e.g., DLL1 or JAG1, for example), a selectin (e.g., E-selectin, P-selectin, L-selectin, for example), Nckl, a mucin (e.g., Mucl or Muc4, for example), a syndecan (e.g., Syndecan1 or Syndecan2, for example) or a CADM (e.g., CADM1 or CADM2, for example). These proteins have been studied and their sequences have been deposited into NCBI's Genbank data.

In any embodiment, the intracellular domain and/or the transmembrane domain of the engineered cell adhesion molecule has an amino acid sequence that is at least 80%, at least 90%, or at least 95% identical to the sequence of an intracellular domain of a wild-type cell adhesion molecule. Specifically, the intracellular domain of the engineered cell adhesion molecule may have a sequence of at least 200 amino acids, at least 100 amino acids, at least amino acids, or at least 20 amino acids, that is at least 80%, at least 90%, or at least 95% identical to the intracellular domain of a cadherin, (e.g., CDH1, CDH2, CDH3, CDH4, CDH5, CDH6, CDH7, CDH8, CDH9, CDH10, CDH11, CDH12, CDH13, CDH14, CDH15, CDH16, CDH17, CDH18, CDH19, CDH2O, CDH21, CDH22, CDH23, CDH24, CDH25, CDH26, CDH27, CDH28, or CDH29, for example), an integrin beta chain (e.g., ITGB1, ITGB2, ITGB3, ITGB4, ITGB5, ITGB6, ITGB7 or ITGB8, for example), an integrin alpha chain (e.g., ITGA1, ITGA2, ITGA3, ITGA4, ITGA5, ITGA6, ITGA7, ITGA8, ITGA9, ITGA10, ITGA11, for example), a junction adhesion molecule (e.g., JAMA, JAMB or JAMC, for example), a protocadherin (e.g., e.g. pcdhal, pcdhbl, pcdhgl, for example), an immunoglobulin adhesion molecule (e.g., e.g. iCAM, MAdCAM-1, NCAM, or CD2, for example), CD44, a claudin (CDLN1, CDLN2, CDLN3, CDLN4, CDLN5, CDLN6, CDLN7, CDLN8, CDLN9, CDLN10, CDLN11, CDLN12, CDLN13, CDLN14, CDLN15, CDLN16, CDLN17, CDLN18, CDLN19, CDLN20, CDLN21, CDLN22, CDLN23, CDLN24 or CDLN25, for example), neurexin/neuroligin/nectin (e.g., (e.g. Neurexinl, Nectinl or Neuroliginl, for example), Eph or ephrin (e.g. EPHA, EPHB, EFNA or EFNB, for example), a notch ligand (e.g., DLL1 or JAG1, for example), a selectin (e.g., E-selectin, P-selectin, L-selectin, for example), Nckl, a mucin (e.g., Mucl or Muc4, for example), a syndecan (e.g., Syndecan1 or Syndecan2, for example), a CADM (e.g., CADM1 or CADM2) or any of the other proteins listed in Table 1, for example).

How these proteins engage with and apply local control over the cytoskeleton, e.g., to reinforce the linkage and/or and recruit additional cytoskeleton to the area has been well studied. For example, Harburger et al (Journal of Cell Science 2009 122: 159-163) provides a detailed review of the mechanism by which integrins are able to signal from extracellular binding to cytoskeletal reorganization; Liu et al (Journal of Cell Science 2000 113: 3563-3571) describes the structure/function relationship of integrins, particularly details the known binding partners of the intracellular sequence of integrins; Hoffman et al (Quant. Cell Biology 2015 25: 803-814) describes the mechanism of cadherin intracellular signaling and how it is mechanically regulated; Beutel et al (Cell 2019 179: 923-936) describes the phase-separation-based mechanism by which JAMs are able to recruit the scaffold protein ZO-1 and signal; and Lawson et al (Pharmacol Rep. 2009 61: 22-32) describes the intracellular signaling pathways that are activated by ICAM signaling. It is noted that some cadherin/integrin and protocadherin/e-cadherin chimeras retain their ability to signal (Geiger et al J. Cell Science 1992 103, 943-951 and Obato et al J. Cell Science 1995 108: 3765-3773).

In any embodiment, the intracellular domain may be selected from Table 1 below, where the amino acid sequence of the intracellular domain may be at least 80%, at least 90%, at least 95% or identical to a human sequence found in Table 1. In these embodiments, the intracellular domain may be an intracellular domain of a cell adhesion molecule selected from Table 1, or a variant thereof that retains the ability to engage with the cytoskeleton

In some embodiments, the intracellular domain may be from an engulfment receptor. In other embodiments, the intracellular domain may be not from an engulfment receptor (i.e., a naturally occurring engulfment receptor or a variant thereof that has at least 90% or 95% sequence identity to the intracellular domain of a naturally-occurring engulfment receptor). In some embodiments, binding of the fusion protein to a second binding moiety does not induce phagocytosis. In these embodiments, the intracellular domain may be from an engulfment receptor but the cell does not phagocytose when the fusion protein binds to its partner. The cell may be incapable of phagocytoses in some cases. For example, in some embodiments the amino acid sequence of the intracellular domain may be less than 80%, less than 90%, or less than 95% an intracellular signaling domain of MegflO, FcRy, Bail, MerTK, TIM4, Stabilin-1, Stabilin-2, RAGE, CD300f, Integrin subunit av, Integrin subunit β5, CD36, LRP1, SCARF1, ClQa, or Axl, or any other engulfment receptor.

Wild-type human cell adhesion molecules associated with their GenBank EntrezlDs are set forth in Table 1 below. The sequences of these molecules are incorporated by reference to their EntrezlDs, in the form that they are in on Oct. 31, 2021. The intracellular and intracellular domains of these proteins should be readily identified because the transmembrane domain is straightforward to identify in these molecules. Many mammalian orthologs of these proteins exist.

TABLE 1

| Protein | EID |
|---|---|
| CDH1 | 999 |
| SEMA3D | 223117 |
| ADGRB1 | 575 |
| ADGRB2 | 576 |
| ADGRB3 | 577 |
| ADGRD1 | 283383 |
| ADGRD2 | 347088 |
| ADGRE1 | 2015 |
| ADGRE2 | 30817 |
| ADGRE3 | 84658 |
| ADGRE5 | 976 |
| ADGRF1 | 266977 |
| ADGRF2 | 222611 |
| ADGRF3 | 165082 |
| ADGRF4 | 221393 |
| ADGRF5 | 221395 |
| ADGRG1 | 9289 |
| ADGRG2 | 10149 |
| ADGRG3 | 222487 |
| ADGRG4 | 139378 |
| ADGRG5 | 221188 |
| ADGRG6 | 57211 |
| ADGRG7 | 84873 |
| ADGRL1 | 22859 |
| ADGRL2 | 23266 |
| ADGRL3 | 23284 |
| ADGRV1 | 84059 |
| AGER | 177 |
| AJAP1 | 55966 |
| ALCAM | 214 |
| AMIGO1 | 57463 |
| AMIGO2 | 347902 |
| AMIGO3 | 386724 |
| ANTXR1 | 84168 |
| ANTXR2 | 118429 |
| ANTXRL | 195977 |
| ASTN1 | 460 |
| ASTN2 | 23245 |
| ATRN | 8455 |
| ATRNL | 26033 |
| AXL | 558 |
| BCAM | 4059 |
| BOC | 91653 |
| BSG | 682 |
| BVES | 11149 |
| CADM1 | 23705 |
| cadm2 | 253559 |
| CADM3 | 57863 |
| cadm4 | 199731 |
| CD151 | 977 |
| CD164 | 8763 |
| CD2 | 914 |
| CD200 | 4345 |
| CD22 | 933 |
| CD226 | 10666 |
| cd244 | 51744 |
| CD300A | 11314 |
| CD302 | 9936 |
| CD33 | 945 |
| CD34 | 947 |
| CD36 | 948 |
| CD4 | 920 |
| CD40LG | 959 |
| CD44 | 960 |
| CD47 | 961 |
| CD48 | 962 |
| CD58 | 965 |
| CD6 | 923 |
| CD72 | 971 |
| CD84 | 8832 |
| CD8A | 925 |
| CD9 | 928 |
| CD93 | 22918 |
| CD96 | 10225 |
| CD99 | 4267 |
| CD99L2 | 83692 |
| CDH10 | 1008 |
| CDH11 | 1009 |
| CDH12 | 1010 |
| CDH13 | 1012 |
| CDH15 | 1013 |
| CDH16 | 1014 |
| CDH17 | 1015 |
| CDH18 | 1016 |
| CDH19 | 28513 |
| CDH2 | 1000 |
| CDH20 | 28316 |
| CDH21 | 26025 |
| CDH22 | 64405 |
| CDH23 | 64072 |
| CDH24 | 64403 |
| CDH25 | 8642 |
| CDH26 | 60437 |
| CDH3 | 1001 |
| CDH4 | 1002 |
| CDH5 | 1003 |
| CDH6 | 1004 |
| CDH7 | 1005 |
| CDH8 | 1006 |
| CDH9 | 1007 |
| CDHR1 | 92211 |
| CDHR2 | 54825 |
| CDHR3 | 222256 |
| CDHR4 | 389118 |
| CDHR5 | 53841 |
| CDON | 50937 |
| CEACAM1 | 634 |
| CEACAM16 | 388551 |
| CEACAM18 | 729767 |
| CEACAM19 | 56971 |
| CEACAM2 | 26367 |
| CEACAM20 | 125931 |
| CEACAM21 | 90273 |
| CEACAM3 | 1084 |
| CEACAM4 | 1089 |
| CEACAM5 | 1048 |
| CEACAM6 | 4680 |
| CEACAM7 | 1087 |
| CEACAM8 | 1088 |
| CELSR1 | 9620 |
| CELSR2 | 1952 |
| CELSR3 | 1951 |
| CERCAM | 51148 |
| CHL1 | 10752 |
| CLCA2 | 9635 |
| CLDN1 | 9076 |
| CLDN10 | 9071 |
| CLDN11 | 5010 |
| CLDN12 | 9069 |
| CLDN14 | 23562 |
| CLDN15 | 24146 |
| CLDN16 | 10686 |
| CLDN17 | 26285 |
| CLDN18 | 51208 |
| CLDN19 | 149461 |
| CLDN2 | 9075 |
| CLDN20 | 49861 |
| CLDN22 | 53842 |
| CLDN23 | 137075 |
| cldn24 | 1E+08 |
| cldn25 | 644672 |
| CLDN3 | 1365 |
| CLDN4 | 1364 |
| CLDN5 | 7122 |
| CLDN6 | 9074 |
| CLDN7 | 1366 |
| CLDN8 | 9073 |
| CLDN9 | 9080 |
| CLEC10a | 10462 |
| CLEC12A | 160364 |
| CLEC12B | 387837 |
| CLEC14A | 161198 |
| CLEC17A | 388512 |
| CLECIA | 51267 |
| clec1b | 51266 |
| CLEC2A | 387836 |
| CLEC2B | 9976 |
| CLEC2D | 29121 |
| CLEC2L | 154790 |
| CLEC4A | 50856 |
| clec4c | 170482 |
| CLEC4D | 338339 |
| CLEC4E | 26253 |
| CLEC4F | 165530 |
| CLEC4G | 339390 |
| CLEC4M | 10332 |
| clec5a | 23601 |
| CLEC6A | 93978 |
| CLEC7A | 64581 |
| CLEC9A | 283420 |
| CLECL1 | 160365 |
| CLMP | 79827 |
| CLSTN1 | 22883 |
| CLSTN2 | 64084 |
| CLSTN3 | 9746 |
| CNTN1 | 1272 |
| CNTN2 | 6900 |
| CNTN3 | 5067 |
| CNTN5 | 53942 |
| CNTN6 | 27255 |
| CNTNAP1 | 8506 |
| CNTNAP2 | 26047 |
| CNTNAP3 | 79937 |
| CNTNAP3B | 728577 |
| CNTNAP4 | 85445 |
| CNTNAP5 | 129684 |
| CRTAM | S6253 |
| CXADR | 1525 |
| DCC | 1630 |
| DCHS2 | 54798 |
| DDR1 | 780 |
| DGCR2 | 9993 |
| DLL1 | 28514 |
| dll3 | 10683 |
| DLL4 | 54567 |
| DSC1 | 1823 |
| DSC2 | 1824 |
| DSC3 | 1825 |
| DSCAM | 1826 |
| DSCAML1 | 57453 |
| DSG1 | 1828 |
| DSG2 | 1829 |
| DSG3 | 1830 |
| DSG4 | 147409 |
| EFNA1 | 1942 |
| efna2 | 1943 |
| efna3 | 1944 |
| efna4 | 1945 |
| efna5 | 1946 |
| EFNB1 | 1947 |
| EFNB2 | 1948 |
| efnb3 | 1949 |
| ELFN1 | 392617 |
| ELFN2 | 114794 |
| EMB | 133418 |
| EMCN | 51705 |
| eng | 2022 |
| EpCAM | 4072 |
| EPHA1 | 2041 |
| EPHA10 | 284656 |
| EPHA2 | 1969 |
| EPHA3 | 2042 |
| EPHA4 | 2043 |
| EPHA5 | 2044 |
| EPHA6 | 285220 |
| EPHA7 | 2045 |
| EPHA8 | 2046 |
| EPHB1 | 2047 |
| EPHB2 | 2048 |
| EPHB3 | 2049 |
| EPHB4 | 2050 |
| EPHB6 | 2051 |
| ESAM | 90952 |
| FAT1 | 2195 |
| FAT2 | 2196 |
| FAT3 | 120114 |
| FAT4 | 79633 |
| FGFRL1 | 53834 |
| FLRT1 | 23769 |
| FLRT2 | 23768 |
| FLRT3 | 23767 |
| FREM1 | 158326 |
| FREM2 | 341640 |
| FREM3 | 166752 |
| GP9 | 2815 |
| GPNMB | 10457 |
| HEPACAM | 220296 |
| ICAM1 | 3383 |
| ICAM2 | 3384 |
| icam3 | 3385 |
| ICAM4 | 3386 |
| ICAM5 | 7087 |
| IGDCC3 | 9543 |
| IGDCC4 | 57722 |
| igsf1 | 3547 |
| igsf10 | 285313 |
| IGSF11 | 152404 |
| IGSF13 | 146722 |
| IGSF16 | 10871 |
| IGSF2 | 9398 |
| IGSF23 | 147710 |
| igsf3 | 3321 |
| IGSF5 | 150084 |
| igsf6 | 10261 |
| igsf8 | 93185 |
| IGSF9 | 57549 |
| IGSF9B | 22997 |
| IL1RAP | 3556 |
| IL1RAPL1 | 11141 |
| IL1RAPL2 | 26280 |
| ITGA1 | 3672 |
| ITGA10 | 8515 |
| ITGA11 | 22801 |
| ITGA2 | 3673 |
| ITGA2B | 3674 |
| ITGA3 | 3675 |
| ITGA4 | 3676 |
| ITGA5 | 3678 |
| ITGA6 | 3655 |
| ITGA7 | 3679 |
| ITGA8 | 8516 |
| ITGA9 | 3680 |
| ITGAD | 3681 |
| ITGAE | 3682 |
| ITGAL | 3683 |
| ITGAM | 3684 |
| ITGAV | 3685 |
| ITGAX | 3687 |
| ITGB1 | 3688 |
| ITGB2 | 3689 |
| ITGB3 | 3690 |
| ITGB4 | 3691 |
| ITGB5 | 3693 |
| ITGB6 | 3694 |
| ITGB7 | 3695 |
| ITGB8 | 3696 |
| JAG1 | 182 |
| JAG2 | 3714 |
| JAMA | 50848 |
| JAMB | 58494 |
| JAMC | 83700 |
| JAML | 120425 |
| KIR2DL1 | 3802 |
| KIR2DL3 | 3804 |
| KIR2DL4 | 3805 |
| KIR2DL5A | 57292 |
| KIR2DL5B | 553128 |
| KIR2DS1 | 3806 |
| KIR2DS2 | 100132285 |
| KIR2DS3 | 3808 |
| KIR2DS4 | 3809 |
| KIR2DS5 | 3810 |
| KIR3DL1 | 3811 |
| KIR3DL3 | 115653 |
| KIR3DS1 | 3813 |
| KIRREL1 | 55243 |
| KIRREL2 | 84063 |
| KIRREL3 | 84623 |
| LICAM | 3897 |
| LAYN | 143903 |
| LILRA1 | 11024 |
| LILRA2 | 11027 |
| LILRA3 | 11026 |
| LILRA4 | 23547 |
| LILRA5 | 353514 |
| LILRA6 | 79168 |
| LILRB1 | 10859 |
| LILRB2 | 10288 |
| LILRB3 | 11025 |
| LILRB4 | 11006 |
| LILRB5 | 10990 |
| LMLN | 89782 |
| LMLN2 | 1B+08 |
| LRFN1 | 57622 |
| LRFN2 | 57497 |
| LRFN3 | 79414 |
| LRFN4 | 78999 |
| LRFN5 | 145581 |
| LRRC4B | 94030 |
| LRRN1 | 57633 |
| LRRN2 | 10446 |
| LRRN3 | 54674 |
| LRRN4 | 164312 |
| LRRN4CL | 221091 |
| LY9 | 4063 |
| LYVE1 | 10894 |
| MADCAM1 | 8174 |
| MAEA | 10296 |
| MAG | 4099 |
| MCAM | 4162 |
| MEGF10 | 84466 |
| MEGF11 | 84465 |
| MEGF9 | 1955 |
| MIA3 | 375056 |
| MOG | 4340 |
| MPL | 4352 |
| MPZ | 4359 |
| MPZL1 | 9019 |
| MPZL2 | 10205 |
| MPZL3 | 196264 |
| MRC1 | 4360 |
| MRC2 | 9902 |
| MTDH | 92140 |
| MUC1 | 4582 |
| MUC12 | 10071 |
| MUC13 | 56667 |
| MUC16 | 94025 |
| MUC17 | 140453 |
| MUC21 | 394263 |
| MUC3A | 4584 |
| MUC4 | 4585 |
| MXRA8 | 54587 |
| NCAM1 | 4684 |
| NCAM2 | 4685 |
| nck1 | 4690 |
| NckAP1 | 10787 |
| NckAP1L | 3071 |
| Nectin1 | 5818 |
| Nectin2 | 5819 |
| Nectin3 | 25945 |
| Nectin4 | 81607 |
| NEO1 | 4756 |
| NFASC | 23114 |
| NINJ1 | 4814 |
| NINJ2 | 4815 |
| NLGN1 | 22871 |
| NLGN2 | 57555 |
| NLGN3 | 54413 |
| NLGN4X | 57502 |
| NLGN4Y | 22829 |
| NPHS1 | 4868 |
| NPTN | 27020 |
| NRCAM | 4897 |
| NRG1 | 3084 |
| NRP1 | 8829 |
| NRP2 | 8828 |
| NRXN1 | 9378 |
| NRXN2 | 9379 |
| NRXN3 | 9369 |
| OCLN | 1.01E+08 |
| OPCML | 4978 |
| PCDH1 | 5097 |
| PCDH10 | 57575 |
| PCDH11X | 27328 |
| PCDH11Y | 83259 |
| PCDH12 | 51294 |
| PCDH15 | 65217 |
| PCDH17 | 27253 |
| PCDH18 | 54510 |
| PCDH19 | 57526 |
| PCDH20 | 64881 |
| PCDH7 | 5099 |
| PCDH8 | 5100 |
| PCDH9 | 5101 |
| PCDHA1 | 56147 |
| PCDHA10 | 56139 |
| PCDHA11 | 56138 |
| PCDHA12 | 56137 |
| PCDHA13 | 56136 |
| PCDHA2 | 56146 |
| PCDHA3 | 56145 |
| PCDHA4 | 56144 |
| PCDHA5 | 56143 |
| PCDHA6 | 56142 |
| PCDHA7 | 56141 |
| PCDHA8 | 56140 |
| PCDHA9 | 9752 |
| PCDHAC1 | 56135 |
| PCDHAC2 | 56134 |
| PCDHB1 | 29930 |
| PCDHB10 | 56126 |
| PCDHB11 | 56125 |
| PCDHB12 | 56124 |
| PCDHB13 | 56123 |
| PCDHB14 | 56122 |
| PCDHB15 | 56121 |
| PCDHB16 | 57717 |
| PCDHB18 | 54660 |
| PCDHB2 | 56133 |
| PCDHB3 | 56132 |
| PCDHB4 | 56131 |
| PCDHB5 | 26167 |
| PCDHB6 | 56130 |
| PCDHB7 | 56129 |
| PCDHB8 | 56128 |
| PCDHB9 | 56127 |
| PCDHGA1 | 56114 |
| PCDHGA10 | 56106 |
| PCDHGA11 | 56105 |
| PCDHGA2 | 56113 |
| PCDHGA3 | 56112 |
| PCDHGA4 | 56111 |
| PCDHGA5 | 56110 |
| PCDHGA6 | 56109 |
| PCDHGA7 | 56108 |
| PCDHGA8 | 9708 |
| PCDHGA9 | 56107 |
| PCDHGB1 | 56104 |
| PCDHGB2 | 56103 |
| PCDHGB3 | 56102 |
| PCDHGB4 | 8641 |
| PCDHGB5 | 56101 |
| PCDHGB6 | 56100 |
| PCDHGB7 | 56099 |
| PCDHGC3 | 5098 |
| PCDHGC4 | 56098 |
| PCDHGC5 | 56097 |
| PDPN | 10630 |
| PECAM1 | 5175 |
| PKHD1 | 5314 |
| plxna1 | 5361 |
| plxna2 | 5362 |
| plxna3 | 55558 |
| plxna4 | 91584 |
| PLXNB1 | 5364 |
| plxnb2 | 23654 |
| PLXNB3 | 5365 |
| PLXNC1 | 10154 |
| plxnd1 | 23129 |
| PODXL | 5420 |
| PODXL2 | 50512 |
| POSTN | 10631 |
| PRPH | 5630 |
| PRPH2 | 5961 |
| PRTG | 283659 |
| PTPRA | 5786 |
| PTPRB | 5787 |
| PTPRC | 5788 |
| PTPRD | 5789 |
| PTPRF | 5792 |
| PTPRG | 5793 |
| PTPRH | 5794 |
| PTPRJ | 5795 |
| PTPRK | 5796 |
| PTPRM | 5797 |
| PTPRO | 5800 |
| PTPRQ | 374462 |
| PTPRS | 5802 |
| PTPRT | 11122 |
| PTPRU | 10076 |
| PTPRZ1 | 5803 |
| PVR | 5817 |
| RET | 5979 |
| ROBO1 | 6091 |
| ROBO2 | 6092 |
| ROBO3 | 64221 |
| ROBO4 | 54538 |
| scarf1 | 8578 |
| scarf2 | 91179 |
| SDC1 | 6382 |
| SDC2 | 6383 |
| SDC3 | 9672 |
| SDC4 | 6385 |
| SDK1 | 221935 |
| SDK2 | 54549 |
| SELE | 6401 |
| SELL | 6402 |
| SELP | 6403 |
| SELPLG | 6404 |
| SEMA3A | 10371 |
| SEMA3B | 7869 |
| SEMA3C | 10512 |
| SEMA3E | 9723 |
| SEMA3F | 6405 |
| SEMA3G | 56920 |
| SEMA4A | 64218 |
| SEMA4B | 10509 |
| SEMA4C | 54910 |
| SEMA4D | 10507 |
| SEMA4F | 10505 |
| SEMA4G | 57715 |
| SEMA5A | 9037 |
| SEMA5B | 54437 |
| SEMA6A | 57556 |
| SEMA6B | 10501 |
| SEMA6C | 10500 |
| SEMA6D | 80031 |
| SEMA7A | 8482 |
| SGCA | 6442 |
| SGCB | 6443 |
| SGCD | 6444 |
| SGCE | 8910 |
| SGCG | 6445 |
| SGCZ | 137868 |
| SIGLEC1 | 6614 |
| siglec10 | 89790 |
| siglec11 | 114132 |
| siglec12 | 89858 |
| siglec13 | 732483 |
| SIGLEC14 | 1E+08 |
| SIGLEC15 | 284266 |
| SIGLEC5 | 8778 |
| siglec6 | 946 |
| siglec7 | 27036 |
| siglec8 | 27181 |
| siglec9 | 27180 |
| SIRPA | 140885 |
| SLAMF1 | 6504 |
| slamf6 | 114836 |
| slamf7 | 57823 |
| slamf8 | 56833 |
| slamf9 | 89886 |
| SLITRK1 | 114798 |
| SLITRK2 | 84631 |
| SLITRK3 | 22865 |
| SLITRK4 | 139065 |
| SLITRK5 | 26050 |
| SLITRK6 | 84189 |
| SMAGP | 57228 |
| SPACA1 | 81833 |
| SPACA2 | 56 |
| SPACA3 | 124912 |
| SPACA4 | 171169 |
| SPACA5 | 389852 |
| SPACA6 | 147650 |
| SPACA7 | 122258 |
| SPACA8 | 54586 |
| SPACA9 | 11092 |
| SPAM1 | 6677 |
| SSPN | 8082 |
| STAB1 | 23166 |
| STAB2 | 55576 |
| SUSD3 | 203328 |
| SUSD5 | 26032 |
| SUSD6 | 9766 |
| TEK | 7010 |
| TENM1 | 10178 |
| TENM2 | 57451 |
| TENM3 | 55714 |
| TENM4 | 26011 |
| THSD1 | 55901 |
| TMEFF2 | 23671 |
| TMEM8B | 51754 |
| TMIGD1 | 388364 |
| TMIGD2 | 126259 |
| TMIGD3 | 57413 |
| Trop2 | 4070 |
| UNC5A | 90249 |
| UNC5B | 219699 |
| UNC5C | 8633 |
| UNC5D | 137970 |
| USH2A | 7399 |
| VCAM1 | 7412 |
| VEZT | 55591 |
| ZAN | 7455 |

TABLE 1

| Protein | EID |
|---|---|
| CDH1 | 999 |
| CDH2 | 1000 |
| CDH3 | 1001 |
| CDH4 | 1002 |
| CDH5 | 1003 |
| CDH6 | 1004 |
| CDH7 | 1005 |
| CDH8 | 1006 |
| CDH9 | 1007 |
| CDH10 | 1008 |
| CDH11 | 1009 |
| CDH12 | 1010 |
| CDH13 | 1012 |
| CDH15 | 1013 |
| CDH16 | 1014 |
| CDH17 | 1015 |
| CDH18 | 1016 |
| CDH19 | 28513 |
| CDH20 | 28316 |
| CDH21 | 26025 |
| CDH22 | 64405 |
| CDH23 | 64072 |
| CDH24 | 64403 |
| CDH25 | 8642 |
| CDH26 | 60437 |
| DCHS2 | 54798 |
| CDHR3 | 222256 |
| CDHR4 | 389118 |
| CDHR1 | 92211 |
| CDHR2 | 54825 |
| FAT1 | 2195 |
| FAT2 | 2196 |
| FAT3 | 120114 |
| FAT4 | 79633 |
| CLSTN1 | 22883 |
| CLSTN2 | 64084 |
| CLSTN3 | 9746 |
| RET | 5979 |
| DSG1 | 1828 |
| DSG2 | 1829 |
| DSG3 | 1830 |
| DSG4 | 147409 |
| DSC1 | 1823 |
| DSC2 | 1824 |
| DSC3 | 1825 |
| CELSR1 | 9620 |
| CELSR2 | 1952 |
| CELSR3 | 1951 |
| ITGB1 | 3688 |
| ITGB2 | 3689 |
| ITGB3 | 3690 |
| ITGB4 | 3691 |
| ITGB5 | 3693 |
| ITGB6 | 3694 |
| ITGB7 | 3695 |
| ITGB8 | 3696 |
| ITGA1 | 3672 |
| ITGA2 | 3673 |
| ITGA2B | 3674 |
| ITGA3 | 3675 |
| ITGA4 | 3676 |
| ITGA5 | 3678 |
| ITGA6 | 3655 |
| ITGA7 | 3679 |
| ITGA8 | 8516 |
| ITGA9 | 3680 |
| ITGA10 | 8515 |
| ITGA11 | 22801 |
| ITGAL | 3683 |
| ITGAM | 3684 |
| ITGAV | 3685 |
| ITGAD | 3681 |
| ITGAE | 3682 |
| ITGAX | 3687 |
| JAMA | 50848 |
| JAMB | 58494 |
| JAMC | 83700 |
| JAML | 120425 |
| CLMP | 79827 |
| CXADR | 1525 |
| PCDHA1 | 56147 |
| PCDHA2 | 56146 |
| PCDHA3 | 56145 |
| PCDHA4 | 56144 |
| PCDHA5 | 56143 |
| PCDHA6 | 56142 |
| PCDHA7 | 56141 |
| PCDHA8 | 56140 |
| PCDHA9 | 9752 |
| PCDHA10 | 56139 |
| PCDHA11 | 56138 |
| PCDHA12 | 56137 |
| PCDHA13 | 56136 |
| PCDHB 1 | 29930 |
| PCDHB2 | 56133 |
| PCDHB3 | 56132 |
| PCDHB4 | 56131 |
| PCDHB5 | 26167 |
| PCDHB6 | 56130 |
| PCDHB7 | 56129 |
| PCDHB8 | 56128 |
| PCDHB9 | 56127 |
| PCDHB10 | 56126 |
| PCDHB11 | 56125 |
| PCDHB12 | 56124 |
| PCDHB13 | 56123 |
| PCDHB14 | 56122 |
| PCDHB15 | 56121 |
| PCDHB16 | 57717 |
| PCDHGA1 | 56114 |
| PCDHGA2 | 56113 |
| PCDHGA3 | 56112 |
| PCDHGA4 | 56111 |
| PCDHGA5 | 56110 |
| PCDHGA6 | 56109 |
| PCDHGA7 | 56108 |
| PCDHGA8 | 9708 |
| PCDHGA9 | 56107 |
| PCDHGA10 | 56106 |
| PCDHGA11 | 56105 |
| PCDHGB1 | 56104 |
| PCDHGB2 | 56103 |
| PCDHGB3 | 56102 |
| PCDHGB4 | 8641 |
| PCDHGB5 | 56101 |
| PCDHGB6 | 56100 |
| PCDHGB7 | 56099 |
| PCDHGC3 | 5098 |
| PCDHGC4 | 56098 |
| PCDHGC5 | 56097 |
| PCDH1 | 5097 |
| PCDH7 | 5099 |
| PCDH8 | 5100 |
| PCDH9 | 5101 |
| PCDH10 | 57575 |
| PCDH11X | 27328 |
| PCDH11Y | 83259 |
| PCDH12 | 51294 |
| PCDH15 | 65217 |
| PCDH17 | 27253 |
| PCDH18 | 54510 |
| PCDH19 | 57526 |
| PCDH20 | 64881 |
| ICAM1 | 3383 |
| ICAM2 | 3384 |
| icam3 | 3385 |
| ICAM4 | 3386 |
| ICAM5 | 7087 |
| VCAM1 | 7412 |
| MADCAM1 | 8174 |
| NCAM1 | 4684 |
| NCAM2 | 4685 |
| CD2 | 914 |
| CD58 | 965 |
| CD48 | 962 |
| ALCAM | 214 |
| CD96 | 10225 |
| CD226 | 10666 |
| NRCAM | 4897 |
| PVR | 5817 |
| CD8A | 925 |
| CD200 | 4345 |
| BCAM | 4059 |
| PECAM1 | 5175 |
| MPZL1 | 9019 |
| MPZL2 | 10205 |
| MPZL3 | 196264 |
| NFASC | 23114 |
| EpCAM | 4072 |
| HEPACAM | 220296 |
| MCAM | 4162 |
| CERCAM | 51148 |
| OPCML | 4978 |
| igsf1 | 3547 |
| IGSF2 | 9398 |
| igsf3 | 3321 |
| CADM1 | 23705 |
| IGSF5 | 150084 |
| igsf6 | 10261 |
| igsf8 | 93185 |
| IGSF9 | 57549 |
| IGSF9B | 22997 |
| igsf10 | 285313 |
| IGSF11 | 152404 |
| CD300A | 11314 |
| IGSF13 | 146722 |
| IGSF16 | 10871 |
| IGSF23 | 147710 |
| CD36 | 948 |
| CD44 | 960 |
| CLDN1 | 9076 |
| CLDN2 | 9075 |
| CLDN3 | 1365 |
| CLDN4 | 1364 |
| CLDN5 | 7122 |
| CLDN6 | 9074 |
| CLDN7 | 1366 |
| CLDN8 | 9073 |
| CLDN9 | 9080 |
| CLDN10 | 9071 |
| CLDN11 | 5010 |
| CLDN12 | 9069 |
| CLDN14 | 23562 |
| CLDN15 | 24146 |
| CLDN16 | 10686 |
| CLDN17 | 26285 |
| CLDN18 | 51208 |
| CLDN19 | 149461 |
| CLDN20 | 49861 |
| CLDN22 | 53842 |
| CLDN23 | 137075 |
| cldn24 | 100132463 |
| cldn25 | 644672 |
| ADGRB1 | 575 |
| ADGRB2 | 576 |
| ADGRB3 | 577 |
| ADGRD1 | 283383 |
| ADGRD2 | 347088 |
| ADGRE1 | 2015 |
| ADGRE2 | 30817 |
| ADGRE3 | 84658 |
| ADGRE5 | 976 |
| ADGRF1 | 266977 |
| ADGRF2 | 222611 |
| ADGRF3 | 165082 |
| ADGRF4 | 221393 |
| ADGRF5 | 221395 |
| ADGRG1 | 9289 |
| ADGRG2 | 10149 |
| ADGRG3 | 222487 |
| ADGRG4 | 139378 |
| ADGRG5 | 221188 |
| ADGRG6 | 57211 |
| ADGRG7 | 84873 |
| ADGRL1 | 22859 |
| ADGRL2 | 23266 |
| ADGRL3 | 23284 |
| ADGRV1 | 84059 |
| SDC1 | 6382 |
| SDC2 | 6383 |
| SDC3 | 9672 |
| SDC4 | 6385 |
| OCLN | 100506658 |
| EFNA1 | 1942 |
| efna2 | 1943 |
| efna3 | 1944 |
| efna4 | 1945 |
| efna5 | 1946 |
| EFNB1 | 1947 |
| EFNB2 | 1948 |
| efnb3 | 1949 |
| EPHA1 | 2041 |
| EPHA2 | 1969 |
| EPHA3 | 2042 |
| EPHA4 | 2043 |
| EPHA5 | 2044 |
| EPHA6 | 285220 |
| EPHA7 | 2045 |
| EPHA8 | 2046 |
| EPHA10 | 284656 |
| EPHB1 | 2047 |
| EPHB2 | 2048 |
| EPHB3 | 2049 |
| EPHB4 | 2050 |
| EPHB6 | 2051 |
| NPHS1 | 4868 |
| KIRREL1 | 55243 |
| KIRREL2 | 84063 |
| KIRREL3 | 84623 |
| NRXN1 | 9378 |
| NRXN2 | 9379 |
| NRXN3 | 9369 |
| NLGN1 | 22871 |
| NLGN2 | 57555 |
| NLGN3 | 54413 |
| NLGN4X | 57502 |
| NLGN4Y | 22829 |
| CNTN1 | 1272 |
| CNTN2 | 6900 |
| CNTN3 | 5067 |
| CNTN5 | 53942 |
| CNTN6 | 27255 |
| CNTNAP1 | 8506 |
| CNTNAP2 | 26047 |
| CNTNAP3 | 79937 |
| CNTNAP3B | 728577 |
| CNTNAP4 | 85445 |
| CNTNAP5 | 129684 |
| DLL1 | 28514 |
| d113 | 10683 |
| DLL4 | 54567 |
| JAG1 | 182 |
| JAG2 | 3714 |
| SELE | 6401 |
| SELP | 6403 |
| SELPLG | 6404 |
| SELL | 6402 |
| nck1 | 4690 |
| DSCAM | 1826 |
| DSCAML1 | 57453 |
| Nectin1 | 5818 |
| Nectin2 | 5819 |
| Nectin3 | 25945 |
| Nectin4 | 81607 |
| CD4 | 920 |
| CD34 | 947 |
| SPAM1 | 6677 |
| CD151 | 977 |
| Trop2 | 4070 |
| ESAM | 90952 |
| AMIGO1 | 57463 |
| AMIGO2 | 347902 |
| AMIGO3 | 386724 |
| SMAGP | 57228 |
| SIGLEC1 | 6614 |
| CD33 | 945 |
| MAG | 4099 |
| SIGLEC5 | 8778 |
| siglec6 | 946 |
| siglec7 | 27036 |
| siglec8 | 27181 |
| siglec9 | 27180 |
| siglec10 | 89790 |
| siglec11 | 114132 |
| siglec12 | 89858 |
| siglec13 | 732483 |
| SIGLEC14 | 100049587 |
| SIGLEC15 | 284266 |
| CLECL1 | 160365 |
| MRC1 | 4360 |
| MRC2 | 9902 |
| SLAMF1 | 6504 |
| LY9 | 4063 |
| cd244 | 51744 |
| CD84 | 8832 |
| slamf6 | 114836 |
| slamf7 | 57823 |
| slamf8 | 56833 |
| slamf9 | 89886 |
| TENM1 | 10178 |
| TENM2 | 57451 |
| TENM3 | 55714 |
| TENM4 | 26011 |
| FLRT1 | 23769 |
| FLRT2 | 23768 |
| FLRT3 | 23767 |
| CHL1 | 10752 |
| LICAM | 3897 |
| BOC | 91653 |
| NPTN | 27020 |
| DCC | 1630 |
| UNC5A | 90249 |
| UNC5B | 219699 |
| UNC5C | 8633 |
| UNC5D | 137970 |
| ASTN1 | 460 |
| ASTN2 | 23245 |
| CD47 | 961 |
| SIRPA | 140885 |
| CD93 | 22918 |
| CD99 | 4267 |
| CD99L2 | 83692 |
| plxna1 | 5361 |
| plxna2 | 5362 |
| plxna3 | 55558 |
| plxna4 | 91584 |
| PLXNB1 | 5364 |
| plxnb2 | 23654 |
| PLXNB3 | 5365 |
| PLXNC1 | 10154 |
| plxnd1 | 23129 |
| scarf1 | 8578 |
| scarf2 | 91179 |
| CD22 | 933 |
| LILRA1 | 11024 |
| LILRA2 | 11027 |
| LILRA3 | 11026 |
| LILRA4 | 23547 |
| LILRA5 | 353514 |
| LILRA6 | 79168 |
| LILRB1 | 10859 |
| LILRB2 | 10288 |
| LILRB3 | 11025 |
| LILRB4 | 11006 |
| LILRB5 | 10990 |
| CD302 | 9936 |
| FGFRL1 | 53834 |
| LRRN1 | 57633 |
| LRRN2 | 10446 |
| LRRN3 | 54674 |
| LRRN4 | 164312 |
| LRRN4CL | 221091 |
| LRFN1 | 57622 |
| LRFN2 | 57497 |
| LRFN3 | 79414 |
| LRFN4 | 78999 |
| LRFN5 | 145581 |
| SPACA1 | 81833 |
| SPACA2 | 56 |
| SPACA3 | 124912 |
| SPACA4 | 171169 |
| SPACA5 | 389852 |
| SPACA6 | 147650 |
| SPACA7 | 122258 |
| SPACA8 | 54586 |
| SPACA9 | 11092 |
| NINJ1 | 4814 |
| NINJ2 | 4815 |
| DGCR2 | 9993 |
| eng | 2022 |
| MUC1 | 4582 |
| MUC3A | 4584 |
| MUC4 | 4585 |
| MUC12 | 10071 |
| MUC13 | 56667 |
| MUC16 | 94025 |
| MUC17 | 140453 |
| CD164 | 8763 |
| NckAP1 | 10787 |
| NckAP1L | 3071 |
| ZAN | 7455 |
| PTPRC | 5788 |
| PTPRD | 5789 |
| PTPRJ | 5795 |
| PTPRK | 5796 |
| PTPRM | 5797 |
| PTPRT | 11122 |
| PTPRU | 10076 |
| SEMA3A | 10371 |
| SEMA3B | 7869 |
| SEMA3C | 10512 |
| SEMA3D | 223117 |
| SEMA3E | 9723 |
| SEMA3F | 6405 |
| SEMA3G | 56920 |
| SEMA4A | 64218 |
| SEMA4B | 10509 |
| SEMA4C | 54910 |
| SEMA4D | 10507 |
| SEMA4F | 10505 |
| SEMA4G | 57715 |
| SEMA5A | 9037 |
| SEMA5B | 54437 |
| SEMA6A | 57556 |
| SEMA6B | 10501 |
| SEMA6C | 10500 |
| SEMA6D | 80031 |
| SEMA7A | 8482 |
| CDON | 50937 |
| CADM3 | 57863 |
| cadm2 | 253559 |
| cadm4 | 199731 |
| DDR1 | 780 |
| IL1RAPL1 | 11141 |
| MAEA | 10296 |
| MEGF10 | 84466 |
| MEGF11 | 84465 |
| MIA3 | 375056 |
| NEO1 | 4756 |
| NRG1 | 3084 |
| POSTN | 10631 |
| PTPRF | 5792 |
| PTPRO | 5800 |
| PTPRS | 5802 |
| SDK1 | 221935 |
| SDK2 | 54549 |
| SSPN | 8082 |
| FREM1 | 158326 |
| FREM2 | 341640 |
| FREM3 | 166752 |
| CD40LG | 959 |
| CD72 | 971 |
| CD9 | 928 |
| AGER | 177 |
| BVES | 11149 |
| ELFN1 | 392617 |
| ELFN2 | 114794 |
| IL1RAP | 3556 |
| IL1RAPL2 | 26280 |
| KIR2DL1 | 3802 |
| KIR2DL3 | 3804 |
| KIR2DL4 | 3805 |
| KIR2DL5A | 57292 |
| KIR2DL5B | 553128 |
| KIR2DS1 | 3806 |
| KIR2DS2 | 100132285 |
| KIR2DS3 | 3808 |
| KIR2DS4 | 3809 |
| KIR2DS5 | 3810 |
| KIR3DL1 | 3811 |
| KIR3DS1 | 3813 |
| KIR3DL3 | 115653 |
| MPL | 4352 |
| NRP1 | 8829 |
| NRP2 | 8828 |
| PODXL | 5420 |
| PODXL2 | 50512 |
| PRPH | 5630 |
| PRPH2 | 5961 |
| PRTG | 283659 |
| ROBO1 | 6091 |
| ROBO2 | 6092 |
| ROBO3 | 64221 |
| ROBO4 | 54538 |
| PTPRB | 5787 |
| PTPRG | 5793 |
| PTPRH | 5794 |
| PTPRQ | 374462 |
| PTPRZ1 | 5803 |
| CEACAM1 | 634 |
| CEACAM2 | 26367 |
| CEACAM3 | 1084 |
| CEACAM4 | 1089 |
| CEACAM5 | 1048 |
| CEACAM6 | 4680 |
| CEACAM7 | 1087 |
| CEACAM8 | 1088 |
| CEACAM16 | 388551 |
| CEACAM18 | 729767 |
| CEACAM19 | 56971 |
| CEACAM20 | 125931 |
| CEACAM21 | 90273 |
| ANTXR1 | 84168 |
| ANTXR2 | 118429 |
| ANTXRL | 195977 |
| AXL | 558 |
| LYVE1 | 10894 |
| SGCA | 6442 |
| SGCB | 6443 |
| SGCD | 6444 |
| SGCE | 8910 |
| SGCG | 6445 |
| SGCZ | 137868 |
| TMEM8B | 51754 |
| USH2A | 7399 |
| VEZT | 55591 |
| CD6 | 923 |
| AJAP1 | 55966 |
| PCDHB18 | 54660 |
| PCDHAC1 | 56135 |
| PCDHAC2 | 56134 |

Exemplary families of intracellular domains from wild-type cell adhesion molecules are set forth in Table 2 below:

TABLE 2

| | intracellular signaling domains from cell adhesion molecules |
|---|---|
| 1. | Cadherins (e.g. CDH1, CDH2 . . . CDH29) (i.e. CDH 1 |
| | through 29) |
| 2. | Cadherin related family-CDHR (CDHR1-16) |
| 3. | Desmoglein (DSG1, DSG2, DSG3, DSG4) |
| 4. | Desmocollin (DSC1, DSC2, DSC3) |
| 5. | Integrin beta chain (e.g. ITGB1, ITGB2 . . . , ITGB8) |
| 6. | Integrin alpha chain (e.g. ITGA1, ITGA2 . . . ITGA11, ITGAE) |
| 7. | Junction Adhesion molecules (e.g. JAMA, JAMB, JAMC, JAML |
| 8. | CXADR |
| 9. | Protocadherins (e.g. pcdha1, pcdhb1, pcdhg1) |
| 10. | Immunoglobulin adhesion molecules (e.g. ICAM1 . . . ICAM5, |
| | VCAM1, MAdCAM-1, NCAM1 . . . NCAM-2, CD2, CD58, |
| | CD48, CD150, ALCAM, CD96, CD226, CD229, NRCAM, |
| | PVR, CD200, BCAM, PECAM1, MPZL2, MAG) |
| 11. | IGSFs (IGSF1-IGSF6, IGSF8, IGSF9, IGSF11-IGSF13, |
| | IGSF16, IGSF23) |
| 12. | CD44 |
| 13. | Claudins (e.g. CLDN1, CLDN2 . . . CLDN25) |
| 14. | Syndecan (SDC1, SDC2, SDC3, SDC4) |
| 15. | Occludin |
| 16. | Ephrins (e.g. EFNA, EFNB) |
| 17. | Eph receptors ( EPHA, EPHB) |
| 18. | Nephrin |
| 19. | Neurexin (NRXN1, NRXN2, NRXN3) |
| 20. | Neuroligin (NLGN1, NLGN2, NLGN3, NLGN4X) |
| 21. | Contactin associated protein (CNTNAP1, CNTNAP2, |
| | CNTNAP3, CNTNAP4, CNTNAP5, CNTNAP3B) |
| 22. | Notch ligands (e.g. DLL1, JAG1) |
| 23. | Selectins (e.g. E-selectin, P-selectin, L-selectin, SELPLG) |
| 24. | SELPLG (PSGL-1) |
| 25. | Nck |
| 26. | DSCAM, DSCAML1 |
| 27. | Nectin |
| 28. | CD4, CD34 |
| 29. | EPCAM, Trop2, ESAM |
| 30. | AMIGO1, AMIGO2, AMIGO3 |
| 31. | SMAGP |
| 32. | CEACAMI-CEACAM21, CERCAM |
| 33. | Lectins (CD209, SIGLEC1-SIGLEC15, CLECL1, MRC1, MRC2 |
| 34. | SLAMF1-9 |
| 35. | MCAM |
| 36. | TENM1-4 |
| 37. | HEPACAM |
| 38. | CD99, CD99L2 |
| 39. | FLRT, FLRT2, FLRT3 |
| 40. | L1 family (NFASC, CHL1, L1CAM, BOC) |
| 41. | BVES |
| 42. | NPTN |
| 43. | Netrin receptor (DCC, UNC5A, UNC5B, UNC5C, UNC5D) |
| 44. | PTPRM |
| 45. | ASTN1 |
| 46. | CD47, SIRPA |
| 47. | CD93 |
| 48. | CD99 |
| 49. | Plexin family (plxna1-4, plxnb1-3, plxnd1) |
| 50. | SCARF1, SCARF2 |
| 51. | CD22 |
| 52. | Leukocyte immunoglobulin like receptor (LILRA1, LILRA2, |
| | LILRA3, LILRA4, LILRA5, LILRA6, LILRB1, |
| | LILRB2, LILRB3, LILRB4, LILRB5) |
| 53. | CD302 |
| 54. | FGFRL1 |
| 55. | Leucine rich repeat neuronal (LRRN1, LRRN2, LRRN3, |
| | LRRN4, LRRN4CL) |
| 56. | Sperm acrosome associated (SPACA1-9) |
| 57. | Calsyntenin (CLSTN1, CLSTN2, CLSTN3) |
| 58. | Ninjurin (NINJ1, NINJ2) |
| 59. | DGCR2 |
| 60. | Endoglin |
| 61. | FGFRL1 |
| 62. | Mucins |
| 63. | NckAP1, NckAP1L |
| 64. | ZAN |

The engineered cell adhesion molecule described above is recombinant in the sense that within a fusion protein the extracellular and intracellular domains are not from the same native (i.e., wild-type) cell adhesion molecule. In some embodiments, the engineered cell adhesion molecule may be a chimeric cell adhesion molecule, where the extracellular and intracellular domains are from different cell adhesion molecules. In any embodiment, the engineered cell adhesion molecule may be a chimeric cell adhesion molecule, where the extracellular and intracellular domains are from the same class of cell adhesion molecules, where the classes are numbered above. In any embodiment, the engineered cell adhesion molecule may be a chimeric cell adhesion molecule, where the extracellular and intracellular domains are from different classes of cell adhesion molecules, where the classes are numbered above. In any embodiment, the fusion may or may not be a fusion between a cadherin family member and an cadherin family member, for example. In any embodiment, the fusion may or may not be a fusion between any of the classes of cell adhesion molecule listed above. In any embodiment, the extracellular binding domain of the fusion protein may not be at least 90% or 95% identical to an extracellular binding domain of any of the adhesion molecules listed in table 1 above, e.g., not an extracellular binding domain of a cadherin selected from CDH1, CDH2 . . . CDH29, etc. or a protocadherin or integrin, etc. (i.e., the extracellular binding domain of naturally occurring adhesion molecule of Table 1 or a variant thereof that has at least 90% or 95% sequence identity to the extracellular domain of a naturally-occurring naturally occurring adhesion molecule).

In any embodiment, the engineered cell adhesion molecule described above may not be not a chimeric antigen receptor; it may not have an intracellular T-cell activation domain (e.g., an ITAM) or co-stimulatory domain and it does not itself activate immune cells upon binding to the second binding moiety. In any embodiment there is no CD3-zeta cytoplasmic domain and no signaling domains from a co-stimulatory molecule, e.g., CD28, CD27, CD134 (OX40), or CD137 in these molecules. Likewise, in any embodiment the engineered cell adhesion molecule is not cleaved to release the intracellular domain by binding to the second binding moiety. In any embodiment, this molecule is not a BTTS (“synNotch”) receptor and does not contain a Notch regulatory region comprising a Lin 12-Notch repeat, an S2 proteolytic cleavage site, or a transmembrane domain comprising an S3 proteolytic cleavage site. Further, the engineered cell adhesion molecule may not be a chimera between two cadherins, integrins or protocadherin, as described above and, in many embodiments, may not have an intracellular domain containing an antibody or DNA binding protein. However, other domains may be present on the molecule. In some embodiments, the cell is not an immune cell and, as such, is incapable of being activated (immune activated) even if the engineered molecule did contain all the necessary motifs for immune cell activation).

Host cells genetically modified with a nucleic acid comprising a nucleotide sequence encoding an engineered cell adhesion molecule as described above are also provided. In some cases, the cell is a eukaryotic cell. In some cases, the cell is a mammalian cell, an amphibian cell, a reptile cell, an avian cell, or a plant cell. In some cases, the cell is a plant cell. In some cases, the cell is a mammalian cell. In some cases, the cell is a human cell. In some cases, the cell is a mouse cell. In some cases, the cell is rat cell. In some cases, the cell is non-human primate cell. In some cases, the cell is lagomorph cell. In some cases, the cell is an ungulate cell. In any embodiment, the fusion protein may not induce phagocytosis of the host mammalian cell when it binds to the second binding moiety that is on another cell or scaffold. However, in some embodiments (depending on the fusion protein, the fusion protein may not induce phagocytosis of the host mammalian cell).

In some cases, the cell is an immune cell, e.g., a T cell, a B cell, a macrophage, a dendritic cell, a natural killer cell, a monocyte, etc. In some cases, the cell is a T cell. In some cases, the cell is a cytotoxic T cell (e.g., a CD8+ T cell). In some cases, the cell is a helper T cell (e.g., a CD4+ T cell). In some cases, the cell is a regulatory T cell (“Treg”). In some cases, the cell is a B cell. In some cases, the cell is a macrophage. In some cases, the cell is a dendritic cell. In some cases, the cell is a peripheral blood mononuclear cell. In some cases, the cell is a monocyte. In some cases, the cell is a natural killer (NK) cell. In some cases, the cell is a CD4+, FOXP3+ Treg cell. In some cases, the cell is a CD4+, FOXP3− Treg cell. In these embodiments, the fusion protein, when expressed in the immune cell, may not activate the cell or induce phagocytosis when it binds to the second binding moiety that is on another cell or scaffold. However, in some embodiments (depending on the fusion protein, the fusion protein may activate the cell or induce phagocytosis when it binds to the second binding moiety that is on another cell or scaffold).

In some instances, the cell is obtained from an individual. For example, in some cases, the cell is a primary cell. As another example, the cell is a stem cell or progenitor cell obtained from an individual. In some instances, the cell may be allogeneic.

As one non-limiting example, in some cases, the cell is an immune cell obtained from an individual. As an example, the cell can be a T lymphocyte obtained from an individual. As another example, the cell is a cytotoxic cell (e.g., a cytotoxic T cell) obtained from an individual. As another example, the cell can be a helper T cell obtained from an individual. As another example, the cell can be a regulatory T cell obtained from an individual. As another example, the cell can be an NK cell obtained from an individual. As another example, the cell can be a macrophage obtained from an individual. As another example, the cell can be a dendritic cell obtained from an individual. As another example, the cell can be a B cell obtained from an individual. As another example, the cell can be a peripheral blood mononuclear cell obtained from an individual.

In some cases, the host cell is not an immune cell. In these embodiments, the host cell may be a somatic cell, e.g. a fibroblast, a hematopoietic cell, a neuron, a pancreatic cell, a muscle cell, a bone cell, a hepatocyte, a pancreatic cell, an epithelial cell, an endothelial cell, a cardiomyocyte, a T cell, a B cell, an osteocyte, or a step cell, and the like.

In addition to the engineered cell adhesion molecule, the cell may also express a therapeutic protein, where the therapeutic protein may be on the surface of the cell, secreted by the cell, in the inside of the cell (e.g., in the cytoplasm or nucleus of the cell).

For example, in some embodiments, the therapeutic protein may be a protein that, when expressed on the surface of an immune cell, activates the immune cell or inhibits activation of the immune cell when it binds to another antigen, e.g., on the diseased cell. In these embodiments, the therapeutic protein may be a chimeric antigen receptor (CAR) or a T cell receptor (TCR). In these embodiments, the cell may additionally comprise an expression cassette comprising: (i) a promoter, and (ii) a coding sequence encoding a CAR or TCR, wherein the CAR or TCR comprises an extracellular binding domain, a transmembrane domain, and an intracellular activation domain, wherein the CAR or TCR activates an immune cell or inhibits activation of the immune cell when it binds to the antigen, e.g., on the diseased cell. Alternatively, the therapeutic protein may be an inhibitory immune cell receptor (iICR) such as an inhibitory chimeric antigen receptor (iCAR), wherein binding of the iICR to the third antigen inhibits activation of the immune cell on which the iICR is expressed. Such iICR proteins are described in e.g., WO2017087723, Fedorov et al. (Sci. Transl. Med. 2013 5: 215ra17) and other references cited above, which are incorporated by reference for that description and examples of the same. In some embodiments such an inhibitory immunoreceptor may comprise an intracellular immunoreceptor tyrosine-based inhibition motif (ITIM), an immunoreceptor tyrosine-based switch motif (ITSM), an NpxY motif, or a YXX(1) motif. Exemplary intracellular domains for such molecules may be found in PD1, CTLA4, BTLA, CD160, KRLG-1, 2B4, Lag-3, Tim-3 and other immune checkpoints, for example. See, e.g., Odorizzi and Wherry (2012) J. Immunol. 188:2957; and Baitsch et al. (2012) PLoSOne 7: e30852.

In some embodiments, therapeutic protein may be an antigen-specific therapeutic that is secreted from the cell. For example, the antigen-specific therapeutic may be an antibody that binds to an immune checkpoint inhibitor e.g., an antibody that binds to PD1, PD-L1, PD-L2, CTLA4, TIM3, LAG3 or another immune checkpoint.

Alternatively, the secreted antigen-specific therapeutic may be a bioactive peptide such as a cytokine (e.g., Il-1ra, IL-4, IL-6, IL-10, IL-11, IL-13, or TGF-β, among many others). In some embodiments, the secreted protein may be an enzyme, e.g., a superoxide dismutase for removing reactive oxygen species, or a protease for unmasking a protease activatable antibody (e.g., a pro-body) in the vicinity of a cancer cell.

Alternatively, the therapeutic protein may be a protein that, when expressed, is internal to the cell, such as wild type or mutant SLP76, ZAP70, or Cas9 protein.

A variety of systems are also provided. In some embodiments, a system may comprise: (a) recombinant cell as described above and (b) a second cell comprising an engineered cell adhesion protein comprising: (i) a second extracellular binding domain comprising a second binding moiety to which the first binding moiety specifically binds; (ii) a transmembrane domain; and an intracellular domain that is capable of signaling to the cytoskeleton of the second cell upon binding of the first binding moiety to the second binding moiety. In these embodiments, the recombinant cell and second cell adhere to each other in a process initiated by binding of the first binding moiety to the second binding moiety. The recombinant and second cells may be separate containers or together in the same container. As noted above, the first and second cells may adhere to each other via homotypic interactions (which might cause clumping of cell), heterotypic interactions or a combination of homotypic and heterotypic interactions. Also as noted above, binding between the first binding moiety to the second binding moiety is conditional, e.g., light or chemically inducible. This binding may be direct or indirect, e.g., via a soluble protein or a scaffold.

Given that the genetic code is known, a nucleotide sequence that encodes an engineered cell adhesion protein can be readily determined. In some embodiments, the coding sequence may be codon optimized for expression in mammalian (e.g., human or mouse) cells, strategies for which are well known (see, e.g., Mauro et al, Trends Mol. Med. 2014 20: 604-613 and Bell et al Human Gene Therapy Methods 27: 6). As would be understood, the coding sequence may be operably linked to a promoter, which may be inducible, tissue-specific, or constitutive. In some embodiments, the promoter may be activated by an engineered transcription factor that is heterologous to the cell, e.g., a Ga14-, LexA-, Tet-, Lac-, dCas9-, zinc-finger- and TALE-based transcription factors.

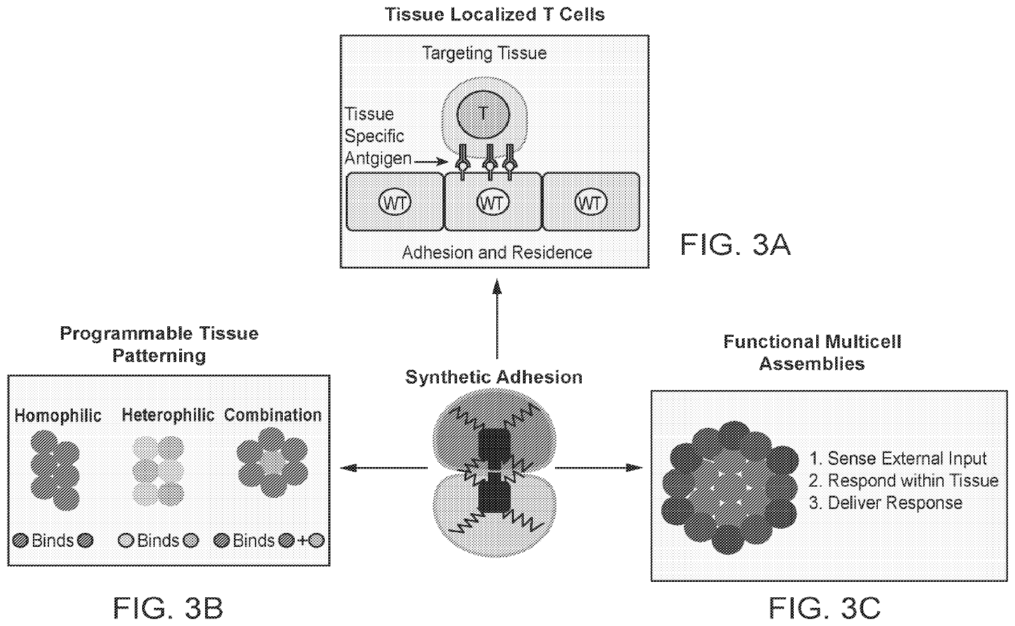

FIG. 3 illustrates some of the uses of the present cells.

In one example (A), the engineered cell adhesion protein may be designed to adhere cells that have a therapeutic payload, e.g., a CAR, antibody, cytokine or enzyme, etc. (as described above), to a particular cell type, tissue or organ. For example, the engineered cell adhesion protein may be designed to adhere cells that have a therapeutic payload to cells of epithelial, connective, muscular, or nervous tissue. In some embodiments, the engineered cell adhesion protein may be designed to adhere cells that have a therapeutic payload to the heart, lungs, liver, kidneys, stomach, intestines, thymus, pancreas, skin, bone, bone marrow, blood, eyes, lymph or lymph nodes, including neurons, pigment cells, cardiac muscle, skeletal muscle, tubule cells, red blood cells, smooth muscle, etc. for example. Cell surface markers associated with such cell types, tissues and organs are known. These embodiments provide a means by which expression of the therapeutic payload can be limited to a particular cell type, tissue or organ, thereby limiting any side effects that may be caused by producing the therapeutic payload in other tissues. These embodiments may provide a method of treatment, wherein the method comprises: administering a composition as described above to a subject, wherein the binding moiety of the engineered cell adhesion protein of the recombinant cell recognizes an antigen on a target cell in the subject and the recombinant cell adheres to the target cell in the subject in vivo. For example, the epitope may be a disease-specific, tissue-specific or organ-specific epitope, for example.

For example, leukocytes could be engineered to become resident within the gut. The ability to specifically control localization of leukocytes to the gut could have applications in both autoimmune diseases, such as Crohn's and Colitis, as well as in the treatment of gastric cancer. For example, Crohn's disease is caused by uncontrolled inflammation within the gut due to a failure of the mucosal immune system (i.e. gut localized immune cells) to host commensal bacteria. Current treatments involve the systematic administration of immunosuppressive biologic drugs (e.g. blocking TNF or alpha-4 integrin), but this treatment can lead to immunosuppression, thereby making those treated more susceptible to infection. One could therefore aim to engineer a synthetic leukocyte that secretes TNF or alpha-4 integrins, but include a synthetic adhesion molecule that targets a gut-specific surface protein (e.g. CDH17). The result should be the establishment of a gut-specific T cells that locally deliver these therapeutic agents, thereby limiting side-effects caused by system immunosuppression.

In another example (B), cell-cell interactions can be customized or pre-programmed to produce tissues that have a defined or semi-defined pattern of cells. For example, the cells within a population of identical cells or a mixed cell population can be adhered to one another via homotypic interactions, heterotypic interactions, or a combination of homotypic and heterotypic interactions. In these embodiments, the method may comprise incubating a composition as described above under conditions by which the cells adhere to each via interactions between the first binding moiety on one cell and ligand for between the first binding moiety on another cell. In some embodiments, the interactions may be homotypic interactions. In some embodiments, the interactions may be heterotypic interactions. In some embodiments, the interactions may be a combination of homotypic and heterotypic interactions. In this utility, the organization of cells within tissue may directly impact the function of the system. For example, this principle is seen in the distinct zones of the lymph node (e.g. B and T cell zones), where interactions between B, T, and dendritic cells are spatially controlled. The ability to design multicellular assemblies with defined patterns therefore has implications in the engineering of synthetic tissue. For example, synthetic adhesion molecules could be applied to generate peripheral lymph nodes with defined lymphocyte organization by engineering stromal, B, T, and/or dendritic cells. These engineered lymph tissue could be applied to either enhance or dampen a local immune response depending on the combination of immune cells and secreted chemokines within the structure.

In another example (C), the engineered cell adhesion protein may facilitate multicell assemblies that are able to sense external stimuli, respond within the tissue, and then deliver a response. In this example, synthetic adhesion molecules could be applied to engineer cells to localize to a targeted tissue and organize into a functional multicellular assembly. In this case, the synthetic adhesion molecules would be used both to target the desired tissue (via binding tissue specific antigens) and self organize (by expressing pairs of adhesion molecules in the engineered cells). These multicellular assemblies will function in cohort to respond to external stimuli. For example, T cells could be targeted to the gut and organize into a two layered assembly of cells. The outer layer could be used to detect markers for inflammation (e.g. cytokines) and then signal to the inner layer to respond by secreting effector cytokines or antibodies. Synthetic adhesion molecules would be important both to localize and organize these effector cells.

As noted above, the traction force, strength, size, protrusiveness/contractility of cell-cell, cell-matrix and cell-material interface can be tuned, as can the actual connections. The fusion proteins and cells containing the same can be used to assemble tissues, drive multicellular self-organization (which can be used in tissue assembly, regenerative medicine, tissue repair and building organs and tissues, etc.), as well as to control cell adhesion in vivo. For example, the fusion proteins can be used to control the adhesion of immune cells that have been administered in vivo, thereby allowing trafficking of those cells (including adhesion, homing, retention and recirculation, etc.) to be controlled. For example, the dwell time of therapeutic immune cells at the target site may be increased using synCAMs that bind to antigens that are at that site. In addition, these molecules can be used to control connectivity of any cell type (e.g., neurons, iPSCs, stem cells, endocrine cells, etc.) and could be used in the repair of neurons, nerves, spinal chord etc.) and in the treatment of a variety of disorders that are amenable to cell therapy (e.g., endocrine disorders, etc.).

### EMBODIMENTS

Embodiment 1. A fusion protein comprising:
(i) an extracellular binding domain comprising a first binding moiety that is capable of specific binding to a second binding moiety;
(ii) one or more transmembrane domains; and
(iii) an intracellular domain that is capable of signaling to the cytoskeleton of the cell upon binding of the first binding moiety to the second binding moiety,
wherein the extracellular binding domain and the intracellular binding domain of the fusion protein are not from the same native cell adhesion molecule.

Embodiment 2. The fusion protein of embodiment 1, wherein the first binding moiety is a scFv or nanobody.

Embodiment 3. The fusion protein of embodiment 1 or 2, wherein:
the intracellular domain is not from an engulfment receptor;
the intracellular domain does not contain a co-stimulatory domain or intracellular T-cell activation domain (ITAM);
the fusion protein, when expressed in a cytotoxic immune cell or stem cell, does not induce phagocytosis of the cell when it binds to the second binding moiety that is on another cell or scaffold;
the fusion protein, when expressed in cytotoxic immune cell, does not activate the cell when it binds to the second binding moiety that is on another cell or scaffold; and/or
the extracellular binding domain of the fusion protein is not an extracellular binding domain of a native cell adhesion molecule.

Embodiment 3A. The fusion protein of embodiment 1 or 2, wherein the intracellular domain is not from an engulfment receptor and/or binding of the fusion protein to a second binding moiety does not induce phagocytosis; and/or
the extracellular binding domain of the fusion protein is not an extracellular binding domain of a native cell adhesion molecule.

Embodiment 3. The fusion protein of any prior embodiment, wherein the intracellular domain is an intracellular domain of a cell adhesion molecule selected from Table 1, or a variant thereof that retains the ability to engage with the cytoskeleton.

Embodiment 4. The fusion protein of any prior embodiment, wherein the fusion protein, when expressed in a mammalian cell, engages with the cytoskeleton of the cell when it binds to the second binding moiety that is on another cell or scaffold.

Embodiment 5. The fusion protein of any prior embodiment, wherein the first binding moiety is capable of specifically binding:
(a) to a naturally-occurring protein expressed on the surface of a partner cell;
(b) to a non-naturally-occurring protein expressed on the surface of a partner cell;
(c) to a scaffold molecule or material bearing the cognate ligand, including natural or unnatural extracellular matrix molecules or hydrogels;
(d) to a partner cell via a homophilic interaction;
(e) to partner cells via a heterophilic interaction;
(f) to multiple partner cells or substrates via a multivalent interaction;
(g) to a partner cell or substrate via a chemically inducible interaction;
(h) to a partner cell or substrate via light- or protease-activated interaction; and/or
(i) to multiple partner cells or substrates via tandem recognition domains.

Embodiment 6. The fusion protein of any prior embodiment, wherein the extracellular binding domain comprises a scFv, a nanobody, a sequence that binds to an antibody, a T cell receptor alpha chain, a T cell receptor beta chain, a ligand for a receptor, a ligand for a cell adhesion protein, a leucine zipper dimerization domain, a split intein, a chemically inducible dimerization domain (e.g., FRB/FKBP), a Snaptag/Halo tag domain, or a light-inducible dimerization domain (e.g. CRY2 and CIB1). Please add: membrane binding domain, glycoprotein binding, ECM binding

Embodiment 8. A nucleic acid encoding a fusion protein of any of embodiments 1-7.

Embodiment 9. A recombinant cell comprising a nucleic acid of embodiment 8, wherein the cell expresses the fusion protein.

Embodiment 10. The cell of embodiment 9, wherein the cell is a mammalian cell.

Embodiment 11. The cell of embodiment 8 or 9, wherein the fusion protein does not induce phagocytosis of the mammalian cell when it binds to the second binding moiety that is on another cell or scaffold.

Embodiment 12. The cell of any of embodiments 9-11, wherein the cell is an immune cell selected from a T cell and a natural killer (NK) cell and, optionally, a macrophage.

Embodiment 13. The cell of embodiment 12, the fusion protein, when expressed in the immune cell, does not activate the cell or induce phagocytosis when it binds to the second binding moiety that is on another cell or scaffold.

Embodiment 14. The cell of any of embodiments 9-11, wherein the cell is a stem cell.

Embodiment 15. The cell of embodiment 9 or 10, wherein the cell is a microglial cell, a Kupfler cell, a neuron, an epithelial cell, an endocrine cell, an endothelial cell, a cardiac cell or a muscle cell.

Embodiment 16. A composition comprising a recombinant cell of any of embodiment 9-15 and a growth medium.

Embodiment 17. The composition of embodiment 16, wherein the composition further comprises a second cell, wherein the recombinant cell and the second cell adhere to each other via an interaction that requires binding of the first binding moiety to the second binding moiety.

Embodiment 18. The composition of embodiment 16 or 17, wherein the recombinant cell and the second cell adhere to each other directly via binding of the first binding moiety to a second binding moiety that is on the surface of second cell.

Embodiment 19. The composition of embodiment 17 or 18, wherein the second cell is not recombinant and the second binding moiety is an antigen on the surface of second cell.

Embodiment 20. The composition of embodiment 19, wherein the antigen is a tissue-specific or disease-specific antigen, or an engineered orthogonal binding domain

Embodiment 21. The composition of embodiment 17, wherein the recombinant cell binds to the second cell indirectly via a soluble bridging molecule.

Embodiment 22. The composition of any of embodiments 16-18 and 21, wherein the second cell is recombinant and has on its surface a second engineered cell adhesion protein comprising:
(i) a second extracellular binding domain comprising the second binding moiety;
(ii) a transmembrane domain; and
(iii) an intracellular domain that is capable of signaling to the cytoskeleton of the cell upon binding of the first binding moiety to the second binding moiety;
wherein the recombinant cell and second cell adhere to each other in a process initiated by binding of the first binding moiety to the second binding moiety.

Embodiment 23. The composition of embodiment 22, wherein the first and second cells adhere to each other via a homotypic interaction.

Embodiment 24. The composition of embodiment 22, wherein the first and second cells adhere to each other via a heterotypic interaction.

Embodiment 25. The composition of any of embodiments 22-24, wherein binding between the first binding moiety to the second binding moiety is conditional.

Embodiment 26. The composition of embodiment 25, wherein binding between the first binding moiety to the second binding moiety is light or chemically inducible.

Embodiment 27. The composition of any of embodiments 16-26, wherein the composition further comprises a tissue scaffold and the recombinant cell adheres to the tissue scaffold via binding of the first binding moiety to the scaffold.

Embodiment 28. The composition of any of embodiments 16-27, wherein the extracellular binding domain comprises a scFv, a nanobody, a sequence that binds to an antibody, a T cell receptor alpha chain, a T cell receptor beta chain, a ligand for a receptor, a ligand for a cell adhesion protein, a leucine zipper dimerization domain, a split intein, a chemically indicuble dimerization domain (e.g., FRB/FKBP), a Snaptag/Halo tag domain, or a light-inducible dimerization domain (e.g. CRY2 and CIB1).

Embodiment 29. The composition of any of embodiments 16-28, wherein the extracellular binding domain is a binding domain of an antibody.

Embodiment 30. The composition of embodiment 29, wherein the extracellular binding domain is a scFv or nanobody.

Embodiment 31. The composition of any of embodiments 16-30, wherein the intracellular domain is an intracellular domain of a cell adhesion molecule selected from Table 1, or a variant thereof that retains the ability to engage with the cytoskeleton.

Embodiment 32. The composition of any of embodiments 16-31, wherein the cell is an immune cell.

Embodiment 33. The composition of embodiment 32, wherein the cell is a CAR T cell.

Embodiment 34. The composition of any of embodiments 16-31, wherein the cell is not an immune cell.

Embodiment 35. A method for altering the binding characteristics of a cell, comprising introducing a nucleic acid encoding a fusion protein of any of embodiments 1-7 into the cell, wherein said introducing results in expression of the fusion protein and alteration of the binding characteristics of the cell.

Embodiment 36. The method of embodiment 35, wherein:
(a) the extracellular binding domain binds to a tissue-specific surface molecule and expression of the fusion protein in the cell results in a longer residency in a selected tissue relative to the same cell without the fusion protein;
(b) the extracellular binding domain binds to a disease-specific surface molecule and expression of the fusion protein in the cell results in a longer residency in a diseased tissue relative to the same cell without the fusion protein;
(c) the extracellular binding domain binds to a molecule on the surface of a target cell and
   i increases the formation of multicellular tissues with a defined structure in vitro or in vivo, controls cell sorting based on differential adhesion strengths;
   ii. controls autonomous sorting of cells based on differential adhesion strengths;
   iii. directs the assembly of an organoid in a disease model;
   iv. directs the assembly of an organ or tissue;
   v. directs regeneration of a tissue or organ in vivo;
   vi. assists in the formation of epithelial-like cell assemblies; or
   vii. directs specific cell-cell connectivities, including multicell circuit/communication systems, including neuronal and endocrine multi-cell systems;
(d) enhances, inhibits or modulates the function of other cell-cell interaction molecules and use in engineering multi-antigen target AND or NOT gates;
(e) abrogates disfunctional adhesion; or
(f) directs or enhances phagocytosis of cognate target cells.

Embodiment 37. A method of treatment, comprising:
administering a cell of any of embodiments 9-15 to a subject, wherein the first binding moiety of the recombinant cell recognizes an antigen on a target cell in the subject and the recombinant cell adheres to the target cell in the subject in vivo.

Embodiment 38. The method of embodiment 37, wherein the antigen is a disease-specific or tissue-specific antigen.

Embodiment 39. A method for adhering a cell to a scaffold, comprising:
combining a cell of any of embodiments 9-15 with a tissue scaffold, wherein the first binding moiety of the engineered cell adhesion molecule binds to the scaffold and the recombinant cell adheres to the scaffold.

Embodiment 41. A method for adhering cells to one another, comprising:
incubating a composition of any of embodiments 16-34 under conditions by which the cells adhere to each via interactions between the first binding moiety on one cell and the second binding moiety on another cell.

Embodiment 42. The method of embodiment 41, wherein the interactions comprise homotypic interactions.

Embodiment 43. The method of embodiment 41, wherein the interactions comprise heterotypic interactions.

Embodiment 44. The method of embodiment 41, wherein the interactions are a combination of homotypic and heterotypic interactions.

### EXAMPLES

The following examples are put forth so as to provide those of ordinary skill in the art with a complete disclosure and description of how to make and use the present invention, and are not intended to limit the scope of what the inventors regard as their invention.

### Example 1

### The Strength of Cell-Cell Interactions can be Modulated Using Intracellular Domains from Different Cell Adhesion Molecules

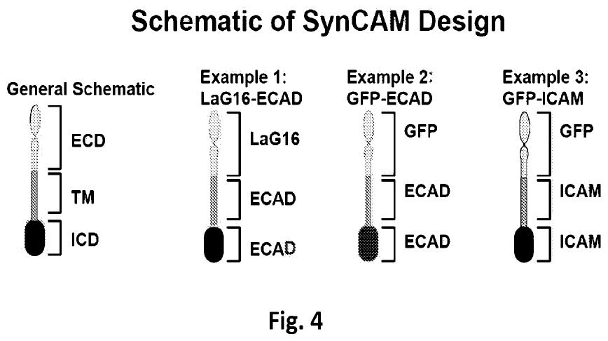

Some of the design principles of the synCAMs used in this example and others are shown in FIG. 4. As shown, the native extracellular domains of, e.g., ICAM and ECAD may be replaced with a binding protein from another protein, e.g., LaG16, which is a nanobody that binds to GFP.

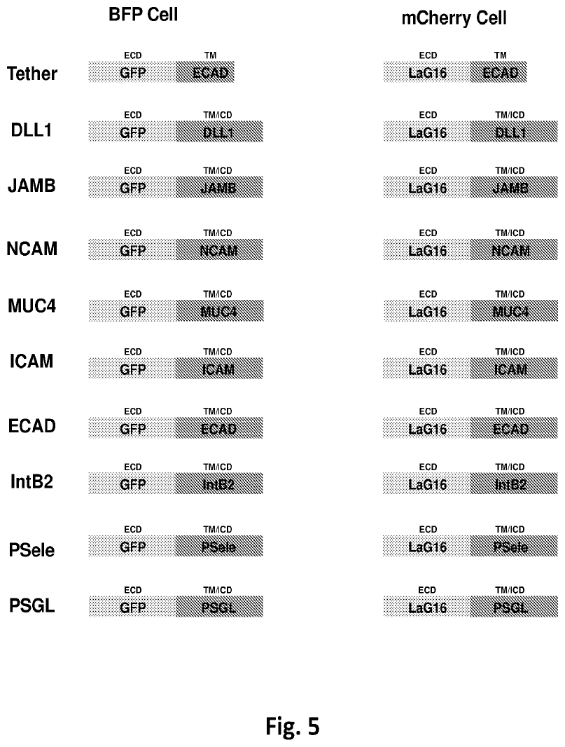

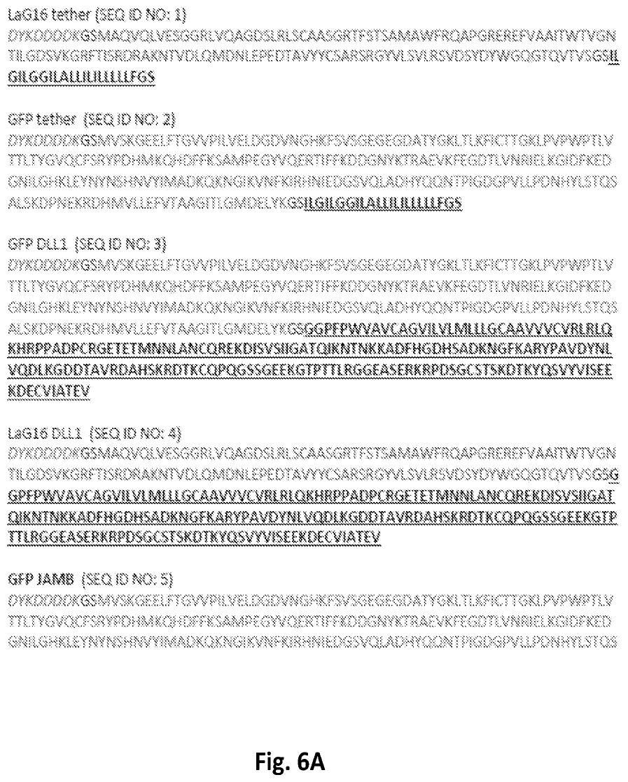

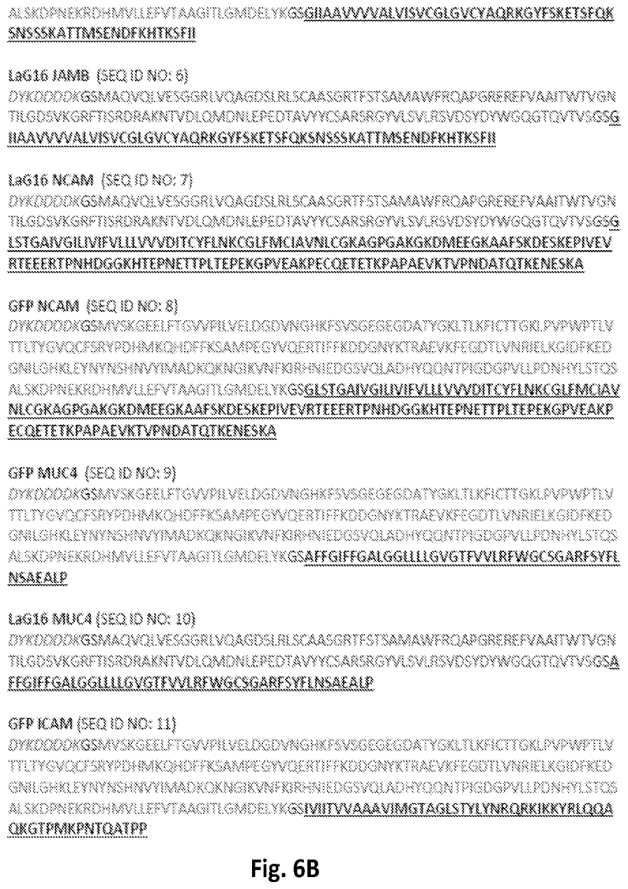

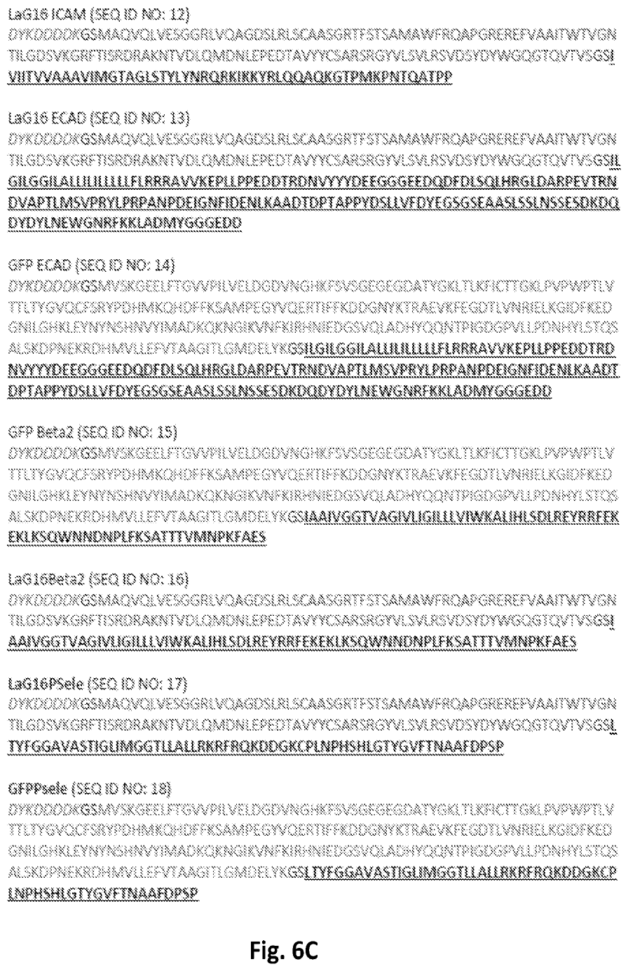

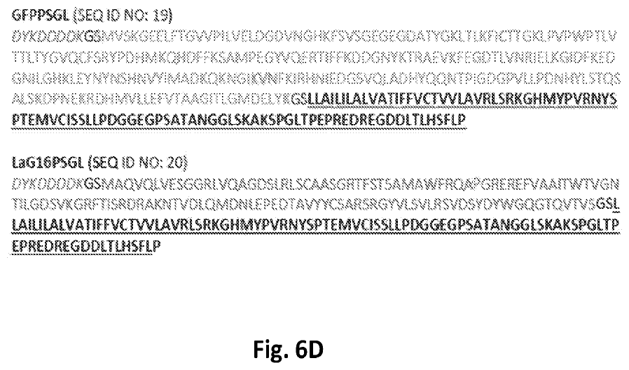

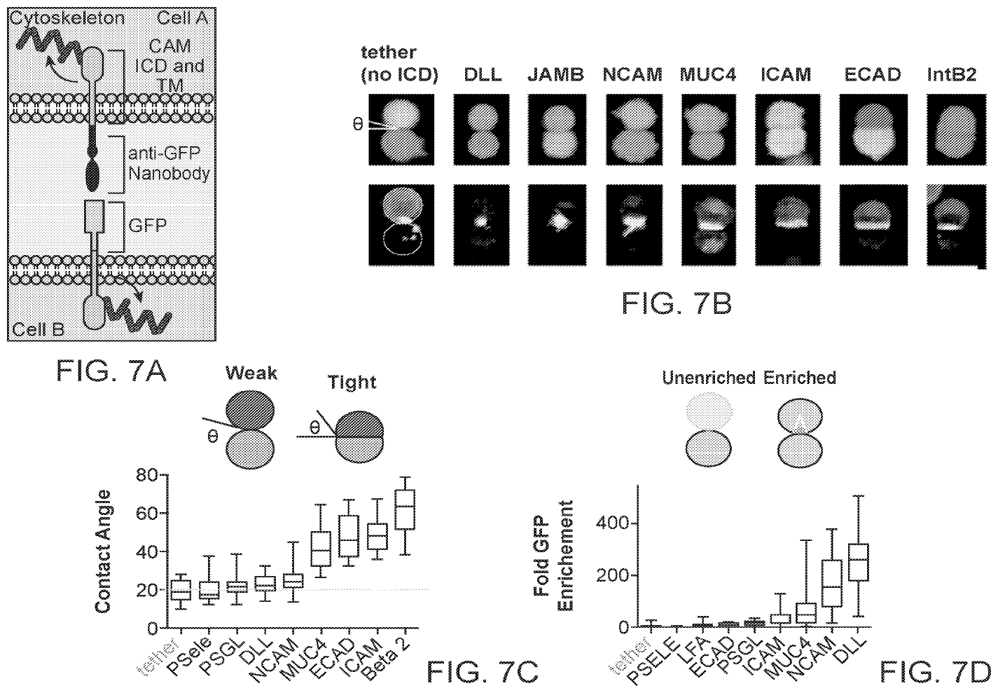

The synCAMs illustrated in FIG. 5 were tested in the following experiments. The sequences of these fusion proteins are shown in FIG. 6. The fusions shown in FIG. 7A were used in some experiments.

Synthetic adhesion receptors were stably integrated into L929 mouse fibroblast cells using lentiviral transduction (400 mL of viral supernatant was added to 1 mL of media (DMEM+10% FBS) and 1E5 L929 cells that constitutively expressed either BFP or mCherry for 24 hours, followed by 48 hours in 1.5 mL media. Cells expressing the indicated synCAM were stained with an aFlag mAb and sorted for surface expression of the receptor (FACS Aria fusion-Beckton-Dickinson). After recovery (˜1 week), the sorted cells were lifted (5 minutes in 1×tryplE), centrifuged (4 min, 400 g) and resuspended in 1 mL of media.

The cells were counted and diluted to a concentration of 5E4/mL. 40 uL of cells expressing complementaryy extracellular recognition domains (GFP and aGFP) for each synCAM were mixed in a 384 ULA flat bottom plate (Greiner). The cells were then imaged in a temperature and environmental controlled chamber (37° C., 5% CO₂) with a high content spinning disc confocal microscope (Opera Phenix). Stacks of images were obtained every hour for up to 5 hours. Typically, 8 replicates for each construct pair were imaged. This data is shown in FIG. 7 B.

FIG. 7B shows individual cell-cell interfaces between cells expressing a synthetic adhesion molecule pair. The important features to note are clear phenotypic differences in the interface between having signaling domains corresponding to adhesion receptors and the tether, which lacks the signaling domain. Furthermore, there are differences between the individual adhesion molecules. The key differences are: (1) the cell morphology, as seen in the contact angle between the two cells. The greater the contact angle, the tighter the interface. (2) Enrichment of GFP at the cell-cell interface. The more GFP is enriched, the more polarized the receptor is. Based off of this data, cell signaling contributes both to the tight interfaces and receptor enriched interfaces.

Contact angles from the confocal images obtained from the images of FIG. 7 B were measured in imageJ. Only cells forming pairwise interfaces were analyzed and maximum projection images were exported from the Phenix Harmony software suite. For each image, the average of the two calculated contact angles was used. Between 10 and 20 replicates for each synCAM was analyzed. The values were plotted as a box and whiskers using Prism software. This data is shown in FIG. 7C.

FIG. 7C quantifies the contact angle in L929 cell pair interface replicates for the indicated synthetic adhesion molecules. This is significant because there is a clear and statistically significant difference in the tighter contact angle mediated by MuC4, ECAD, ICAM, and Beta2 integrin synCAMs compared to the control (tether), as well as the Psele and PSGL synCAMs.

Maximum projection images of the GFP channel were analyzed directly in the phenix harmony software. The ratio of average GFP intensity at the cell-cell interface was divided by the average intensity at the apical face of the cell to determine the fold enrichment. Between 10 and 20 replicates were measured for each indicated synCAM construct. This data is shown in FIG. 7D.

FIG. 7D quantifies GFP enrichment in L929 cell pair interface replicates for the indicated synthetic adhesion molecules. This is significant because there is a clear and statistically significant difference in the degree to which GFP (and therefore the synthetic adhesion receptors) polarize at the interface for MUC4, NCAM, and DLL compared to the control (tether) and other indicated synCAMs.

### Example 2

### The Strength of Cell-Cell Interactions can be Modulated Using Different Extracellular Domains

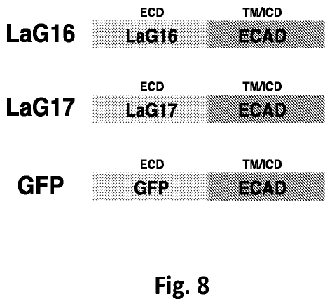

The synCAMs illustrated in FIG. 8 were tested in the following experiments. The sequences of these fusion proteins are shown in FIG. 9.

Synthetic adhesion receptors were stably integrated into L929 mouse fibroblast cells using lentiviral transduction (400 mL of viral supernatant was added to 1 mL of media (DMEM+10% FBS) and 1E5 L929 cells that constitutively expressed either BFP, mCherry, or no fluorescent protein for 24 hours, followed by 48 hours in 1.5 mL media. Cells expressing the indicated synCAM were stained with an aFlag mAb and sorted for surface expression of the receptor (FACS Aria fusion-Beckton-Dickinson). After recovery (˜1 week), the sorted cells were lifted (5 minutes in lx tryplE), centrifuged (4 min, 400 g) and resuspended in 1 mL of media.

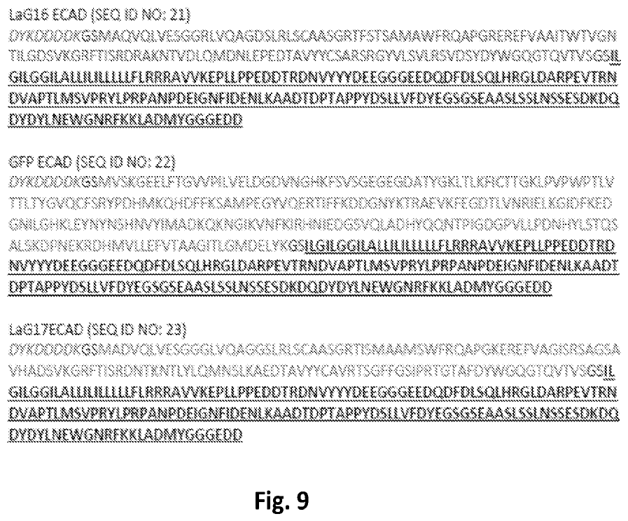

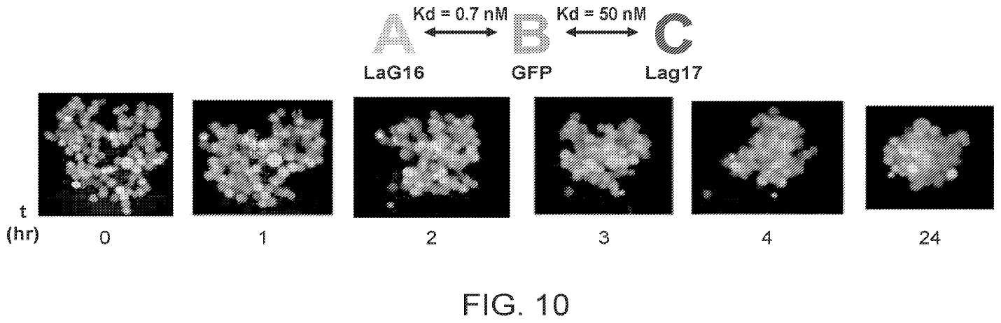

The cells were then counted and diluted to a concentration of 1E3/mL. 27 uL of cells expressing the indicated synCAM (GFP, LaG16, or LaG17) for each synCAM were mixed in a 384 well ultra-low attachment (ULA) round bottom plate (Corning or Greiner) and centrifuged (2 min, 200 g). The cells were then imaged in a temperature and environmental controlled chamber (37° C., 5% CO₂) in a high content spinning disc confocal microscope (Opera Phenix). Stacks of images were obtained every hour for 24 hours. The images shown in FIG. 10 were maximum projections exported from the Phenix Harmony software as a maximum projection at the indicated time point.

FIG. 10 shows that the LaG16-ECAD (orange) and LaG17-ECAD (blue) expressing cells are competing to bind the GFP-ECAD (green) expressing cells. At early time points both LaG16 and LaG17 cells bind efficiently to the GFP cells. However, after 24 hours, interactions between the LaG16 and GFP cells are favored, thereby sorting the LaG17 cells to the exterior of the sphere. This shows that the affinity of the extracellular domain contributes to the strength of the synthetic adhesion interaction. This result is relevant because it shows an additional use for the synthetic adhesion molecules. In FIG. 7A-D, it was shown that the control of the phenotype of the cell-cell interaction is based on signaling properties of the intracellular domain, while in FIG. 10 it is shown that that toggling extracellular affinity can be used to control interaction favorability.

### Example 3

### synCAMS can be Used to Assemble Cells Via Homophilic and Heterophilic Interactions

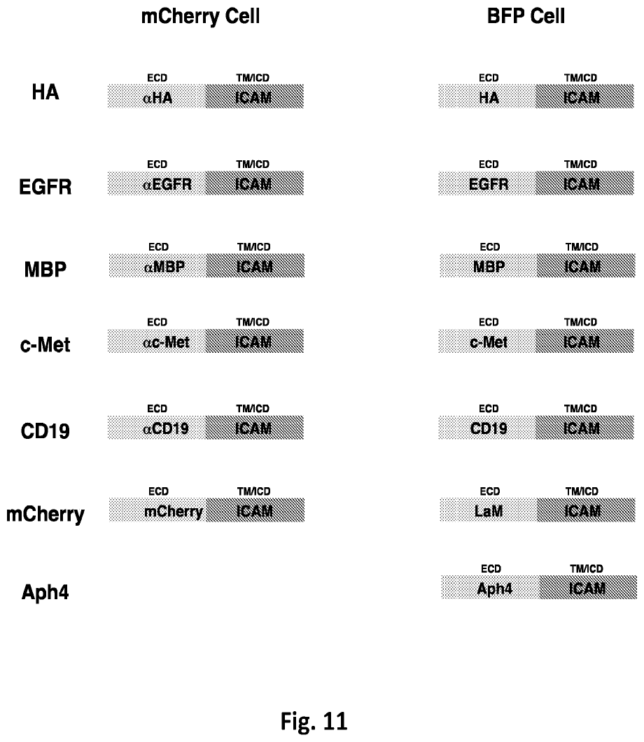

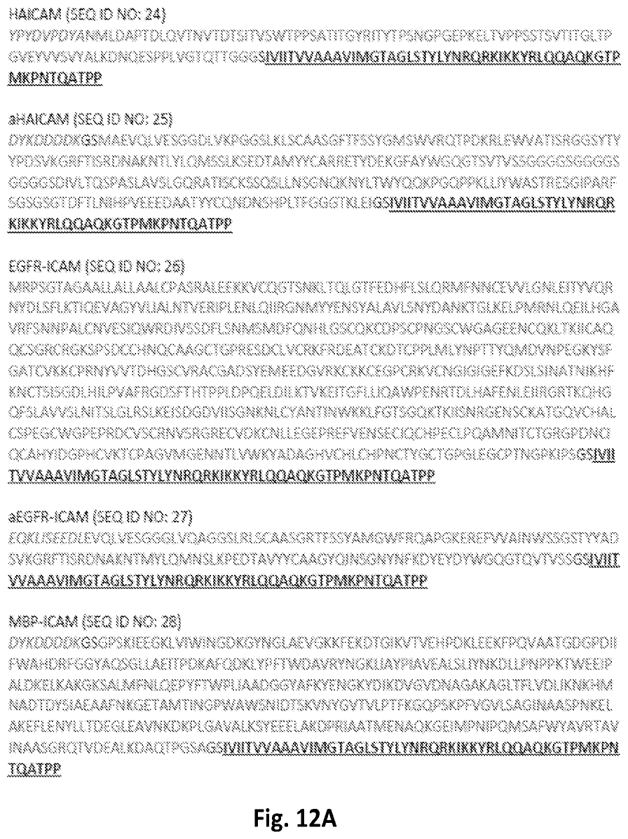

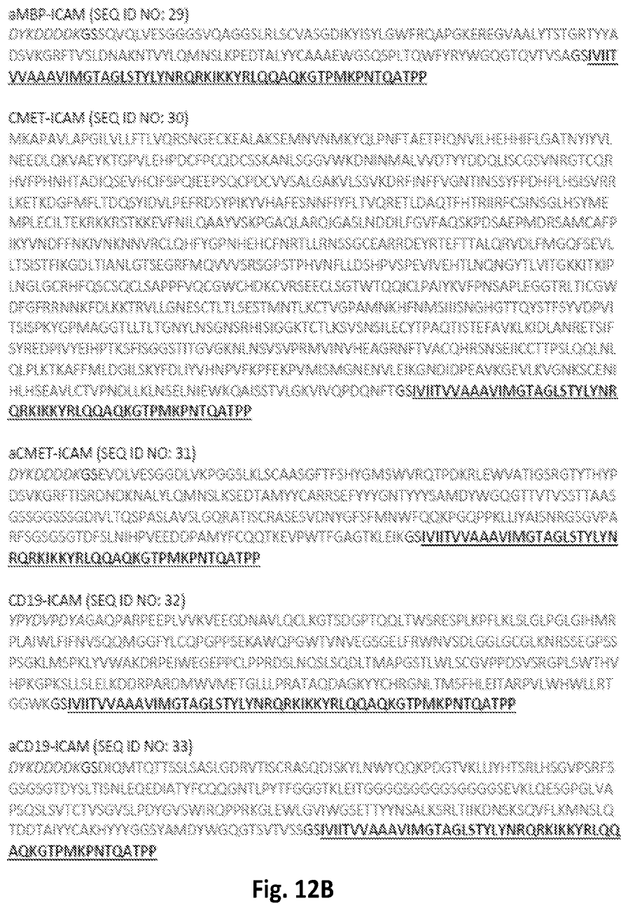

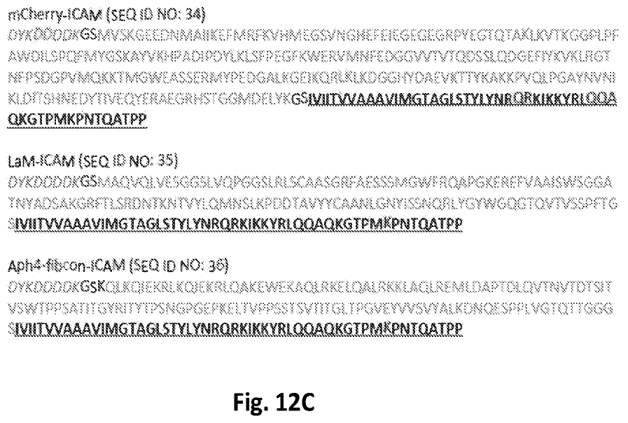

The synCAMs illustrated in FIG. 11 were tested in the following experiments. The sequences of these fusion proteins are shown in FIG. 12.

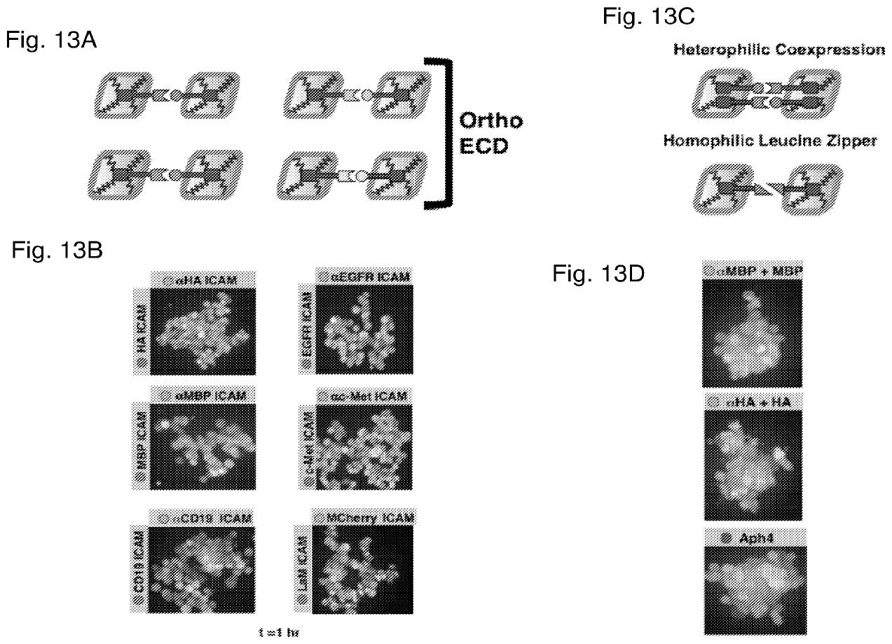

FIG. 13A illustrates synCAM heterophilic extracellular domains; FIG. 13C illustrates two strategies to program synCAM homophilic assemblies.

For the data shown in FIGS. 13B and D, synthetic adhesion receptors were stably integrated into L929 mouse fibroblast cells using lentiviral transduction (400 mL of viral supernatant was added to 1 mL of media (DMEM+10% FBS) and 1E5 L929 cells that constitutively expressed either BFP or mCherry for 24 hours, followed by 48 hours in 1.5 mL media. Cells expressing the indicated synCAM were stained and sorted for surface expression of the receptor (FACS Aria fusion-Beckton-Dickinson). After recovery (˜1 week), the sorted cells were lifted (5 minutes in lx tryplE), centrifuged (4 min, 400 g) and resuspended in 1 mL of media.

The cells were then counted and diluted to a concentration of 1E3/mL. 40 uL of cells expressing the indicated synCAM for each pair were mixed in a 384 well ultra-low attachment (ULA) round bottom plate (Corning or Greiner) and centrifuged (2 min, 200 g). The cells were then imaged in a temperature and environmental controlled chamber (37° C., % CO2) in a high content spinning disc confocal microscope (Opera Phenix). Stacks of images were obtained every hour for up to 24 hours. The images shown in the figure were maximum projections exported from the Phenix Harmony software. This data is shown in FIGS. 13A and C.

FIG. 13A shows that synCAMs can work with multiple different heterophilic protein binding pairs beyond the heterophilic GFP/aGFP pairs shown the earlier examples. This is relevant because it illustrates the programmability of the synCAM technology.

FIG. 13D demonstrate that synCAMs can be used to generate homophilic cellular assemblies-either by coexpression of two heterophilic synCAMs in the same cell or expression of a synCAM containing a homophilic extracellular domain. This is important because being able to dictate both homophilic and heterophilic cellular interactions expands the cellular patterns that can be designed with synCAMs.

### Example 4

### synCAMs can be Used Pattern Cell Assembly

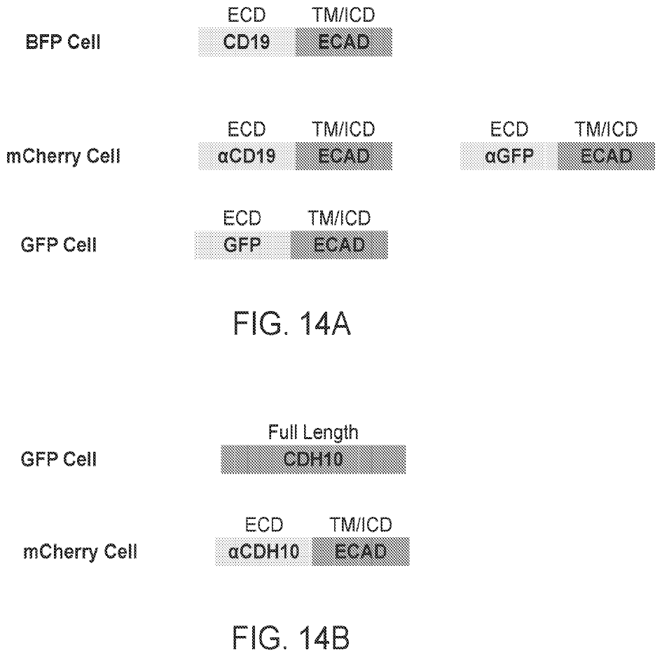

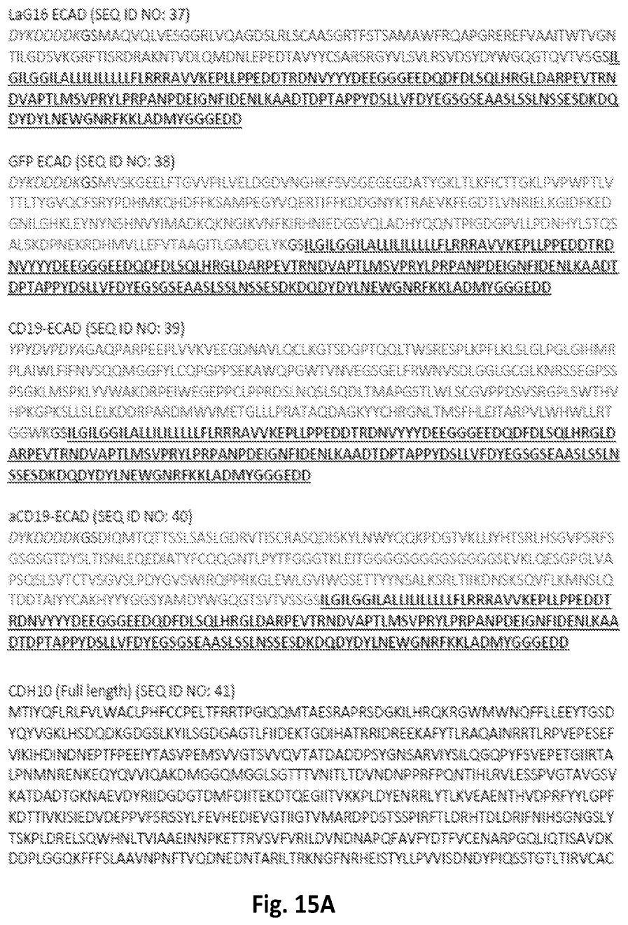

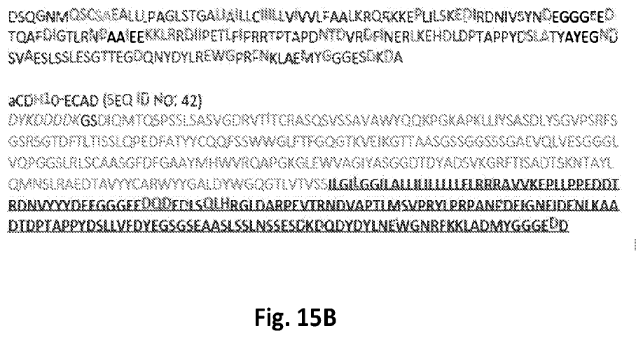

The synCAMs illustrated in FIG. 14 were tested in the following experiments. The sequences of these fusion proteins are shown in FIG. 15.

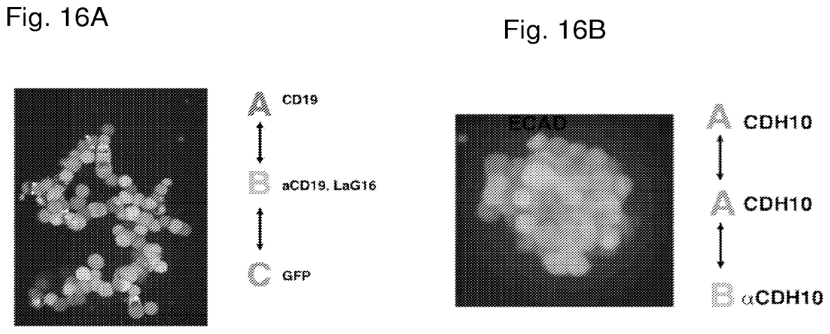

For the data shown in FIG. 16A, aynthetic adhesion receptors were stably integrated into L929 mouse fibroblast cells using lentiviral transduction (400 mL of viral supernatant was added to 1 mL of media (DMEM+10% FBS) and 1E5 L929 cells that constitutively expressed either BFP, mCherry, or no fluorescent protein or 24 hours, followed by 48 hours in 1.5 mL media. Cells expressing the indicated synCAM were stained and sorted for surface expression of the receptor (FACS Aria fusion-Beckton-Dickinson). After recovery (˜1 week), the sorted cells were lifted (5 minutes in lx tryplE), centrifuged (4 min, 400 g) and resuspended in 1 mL of media.

The cells were then counted and diluted to a concentration of 1E3/mL. 27 uL of cells expressing the indicated synCAM for each pair were mixed in a 384 well ultra-low attachment (ULA) round bottom plate (Corning or Greiner) and centrifuged (2 min, 200 g). The cells were then imaged in a temperature and environmental controlled chamber (37° C., % CO₂) in a high content spinning disc confocal microscope (Opera Phenix). Stacks of images were obtained every hour for up to 24 hours. The image shown in FIG. 16A was a maximum projection exported from the Phenix Harmony software.

FIG. 16A demonstrates that the use of two orthogonal synCAMs (in this case one based on CD19/aCD19 and one based on GFP/LaG) can be used to pattern the assembly of cells (in this case forming an A-B-C pattern. This is relevant because it shows that adhesion molecules can be used to design complex tissue architecture.

For the data shown in FIG. 16B, wild type CDH10 or the aCDH10-ECAD adhesion receptors were stably integrated into L929 mouse fibroblast cells using lentiviral transduction (400 mL of viral supernatant was added to 1 mL of media (DMEM+10% FBS) and 1E5 L929 cells that constitutively expressed either mCherry or GFP for 24 hours, followed by 48 hours in 1.5 mL media. Cells expressing the indicated synCAM were stained and sorted for surface expression of the receptor (FACS Aria fusion-Beckton-Dickinson). After recovery (˜1 week), the sorted cells were lifted (5 minutes in lx tryplE), centrifuged (4 min, 400 g) and resuspended in 1 mL of media.

The cells were then counted and diluted to a concentration of 1E3/mL. 40 uL of each of these cells were mixed in a 384 well ultra-low attachment (ULA) round bottom plate (Corning or Greiner) and centrifuged (2 min, 200 g). The cells were then imaged in a temperature and environmental controlled chamber (37° C., 5% CO₂) in a high content spinning disc confocal microscope (Opera Phenix). Stacks of images were obtained every hour for up to 24 hours. The image shown in FIG. 16 B was a maximum projection exported from the Phenix Harmony software.

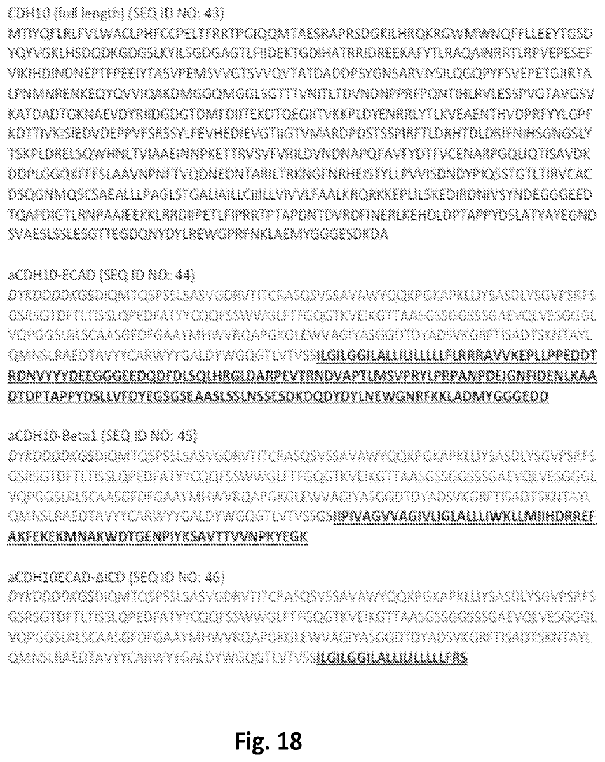

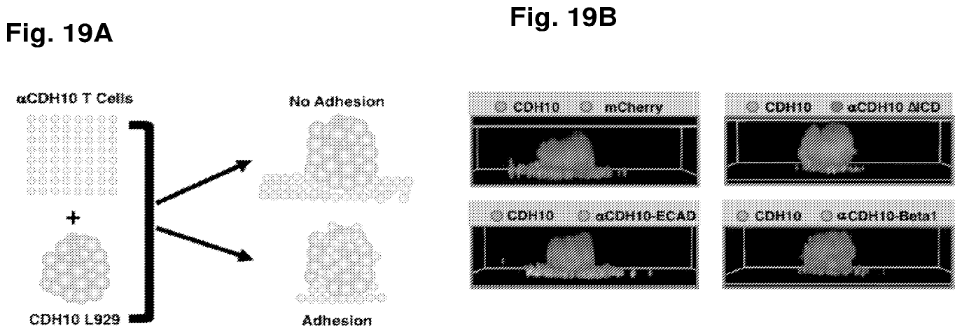

FIG. 18B demonstrates that synCAMs can directly bind endogenous adhesion molecules, in this case the green cells bind homophilically through Cadherin 10, and the orange cells are able to incorporate within this green sphere by expressing a synCAM that directly binds CDH10 (aCDH10). This is significant because it shows that one can build multicellular structures that consist of both endogenous and synthetic adhesion, thereby making these semisynthetic cellular assembly. Furthermore, this experiment lays the foundation for targeting T cells to endogenous adhesion molecules (shown in FIGS. 19A and B).

### Example 5

### synCAMs are Functional in Primary Human T Cells

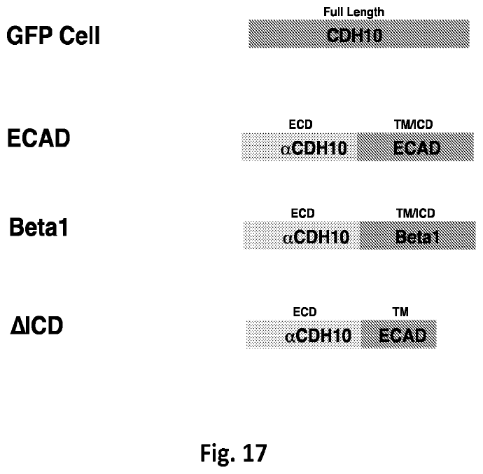

The synCAMs illustrated in FIG. 17 were tested in the following experiments. The sequences of these fusion proteins are shown in FIG. 18.

For the data shown in FIG. 19B, primary T cells were thawed, cultured for 24 hr, and then stimulated with Dynabeads Human T-Activator CD3/CD28 (Life Technologies #11132D) at a 1:1 cell:bead ratio. Primary T cells were exposed to lentiviral transducton vectors containing ocCDH1O-Syncads for 24 hr. Dynabeads were removed at day 4 post T cell stimulation and the T cells were expanded until day 9. The cells were then stained for synCAM expression using a FITC-conjugated ocFlag mAb and sorted with a FACs ARIA II (BD biosciences). The sorted cells were allowed to recover for −3 days and were then counted, diluted, and mixed with L929 fibroblast cells expressing CDH10 in a ULA roundbottom plate (200 T cells with 80 L929 fibroblast cells per assay). After 24 hours, the cells were then imaged with fluorescence spinning disc confocal microscopy (phenix). The images shown in FIG. 19B were exported as maximum projections from the Phenix Harmony software.

The results of FIG. 19B show that synCAMs are able to function in primary human T cells. The aCDH10-Beta1 synCAM enables T cells to bind the aCDH10 target cells better than the aCDH10-ECAD synCAM, mcherry control, and aCDH10ΔICD control (seen by ability of orange cells to stick to green sphere). This is relevant because it shows how primary T cells and other lymphocytes can be engineered to bind to tissues and tumor specific antigens. For example, CDH10 is a brain-specific antigen that could be used to target engineered cells the brain.

### Example 6

### Use of synCAMs to Localize Cells to a Particular Tissue

The following is a prophetic example illustrating how cells can be localized to a particular tissue.

Dysregulation of the mucosal immune response to commensal bacteria in the gastrointestinal tract is hypothesized to be a primary driver of inflammatory bowel disease. By transduction of a synthetic adhesion molecule, lymphocyte cells are engineered to become resident within the gastrointestinal tissue and effect the immune response.

Primary human T cells are transduced with a synCAM expressing a synCAM that targets the gut-specific adhesion molecule CDH17. This synCAM is a chimera between a single chain variable fragment that recognizes CDH17 ectodomain and adhesion molecule (e.g. ICAM, ECAD, Beta1 integrin) transmembrane, and intracellular domain. The function of these cells is first tested in vitro by mixing the synCAM engineered primary T cells (or control constructs lacking the intracellular signaling domain or recognition ectodomain) with L92.9 fibroblast cells transduced with CDH17. Recognition is monitored by confocal microscopy. Next, engineered T cells are prepared expressing both a CDH17 targeting synCAM and luciferase. These cells are injected into an NSG mouse and monitored with the luciferase signal for their ability to hone to and take up residence within the gastrointestinal tract of the gut. Lastly, the ability of these gut-homing T cells to augment the pathology of IBD is tested in a mouse disease model using a syngeneic system in which mouse T cells are engineered with the anti CDH17 synCAMs to localize to the gut, but also secrete a payload that will modulate the immune response. For example, the T cells may secrete cytokines that favor the local formation of T regulatory cells rather than Th17 cells.

### Example 7

### Use of synCAMs to Program Cells for Building Tissues

The following is a prophetic example illustrating how cells can be programmed build a tissue

The organization of cells within tissue directly impacts the function of the system. For example, this principle is seen in the distinct zones of the lymph node (e.g. B and T cell zones), where interactions between B, T, and dendritic cells are spatially controlled. The ability to design multicellular assemblies with defined patterns therefore has implications in the engineering of synthetic tissue.

In this example, synthetic adhesion molecules are used to generate peripheral lymph nodes with defined lymphocyte organization by engineering stromal, B, T, and/or dendritic cells to organize in a tissue specific manner. These peripheral lymph nodes can potentially serve as hubs to control the immune response locally. One example of this strategy relies on designing a tertiary lymphoid structure based on dendritic and T-cells. The T cell expresses two synthetic adhesion molecules: one encoding a tissue specific targeting function (e.g. the gut specific synCAMs outlined above and another encoding an orthogonal synCAM to bind the dendritic cell (e.g. a synCAM with a GFP ectodomain and ICAM TM and ICD). The dendritic cell also would express two synCAMs, one encoding a tissue specific targeting function and another that complements that synCAM expressed in the T cell (e.g. LaG ectodomain fused to ICAM TM and ICD). The adhesion molecules are introduced into these cells using viral transduction, and they are injected into a mouse to characterize the ability to traffic to and organize into structures locally within the gut. This engineered lymphoid tissue is applied to either enhance or dampen a local immune response depending on the combination of immune cells and secreted chemokines within the structure. In the case of autoimmunity, the T cells are engineered to secrete immunomodulatory cytokines such as TGF beta to facilitate the formation of more regulatory T cells within this lymphoid structure and can be tested within a mouse model of gut inflammation.

### Example 8

### Use of synCAMs to Produce Multilayer Assemblies

The following is a prophetic example illustrating how cells can be programmed to build a functional multilayer assembly.

Synthetic adhesion molecules could potentially be applied to engineer cells to localize to a targeted tissue and organize into a functional multicellular assembly. In this case, the synthetic adhesion molecules is used both to target the desired tissue (via binding tissue specific antigens) and self-organize (by expressing pairs of adhesion molecules in the engineered cells). These multicellular assemblies should function in cohort to respond to external stimuli. For example, the ability to precisely control an immune response is critical in organ transplantation. Donor organs can be tolerated in a host and avoid acute rejection due to the systematic administration of immunosuppressive drugs. Nevertheless, rejection of a donor organ typically occurs after a number of years due to a host immune response against the foreign tissue. One contributing factor to organ rejection is the pharmacological limitations of balancing suppression of an immune response targeting the donor tissue with the deleterious consequences of systematic immunosuppression (e.g. pathogen infection or development of cancer).

In this example, regulatory T cells from an organ donor are engineered ex vivo and targeted to the lungs to organize into a two layered assembly of cells as a means to establish peripheral tolerance to a lung transplant. In this example, one group of T cells is transduced to express a synCAM that targets lung-specific adhesion molecule such as CDHR3 (an ectodomain consisting of an anti CDHR3 single chain variable fragment fused to a TM and ICD of an adhesion molecule such as ICAM or ECAD), an orthogonal synCAM to bind the second group of regulatory T cells (e.g. GFP-ICAM), and a synNotch receptor that activates transcription of immunosuppressive cytokines or effectors (e.g. TGF beta). This first group of T cells would establish adhesion directly to the lung tissue. A second set of T cells, which would form the outer layer, would express an adhesion molecule that binds the first set of T cells (e.g. LaG-ICAM), along with sensor receptors to detect the generation of an immune response from the host (these sensors could be versions of synNotch designed to detect inflammatory cytokines such as IL6 or TNFalpha). Importantly, detection of inflammation in the outer layer would lead to expression of the ligand for synNotch in the inner layer, which should activate the internal secretion of immunosuppressive cytokines.

### Example 9

### Systematic Exploration of Engineered Synthetic CAMs (synCAMs) Through Substituting CAM ECDs with Programahle Heterologous Binding. Domains

A collection of transmembrane receptors, termed cellular adhesion molecules (CAMs), has evolved to accomplish the expansive set of biological processes requiring adhesive cell interactions (Cavallaro and Dejana, 2011; Rubinstein et al., 2015; Sun et al., 2016). CAMs transduce a specific extracellular binding event (to a neighboring cell or matrix) into an intracellular signaling response that often involves cytoskeletal reorganization and changes in cell morphology. Examples of CAMs include integrins, which form focal adhesion contacts, cadherins, which form adherens junctions, and junction adhesion molecules (JAMs), which contribute to tight junction formation (Ebnet et al., 2004; Kinashi, 2005; Yap and Kovacs, 2003). These separate families of CAMs employ distinct mechanisms of extracellular ligand binding and activate unique downstream signaling cascades. CAM structural complexity and functional diversity therefore encumbers efforts to design broad synthetic control of cellular adhesion. The extent to which CAMs are modular and amenable to engineering is unknown.

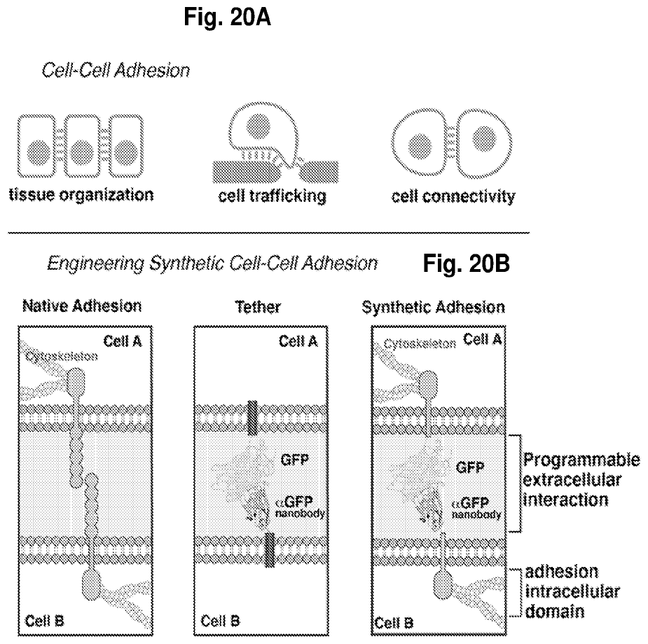

The following examples describe systematic exploration of engineered synthetic CAMs (synCAMs) through substituting CAM ECDs with programable heterologous binding domains (FIG. 20A). Eight different adhesion ICDs were explored: E-cadherin (Ecad), beta 1 integrin (Intβ1), beta 2 integrin (Intβ2), Intercellullar adhesion molecule 1 (ICAM-1), Delta-like protein 1 (DLL1), Junction adhesion molecule B (JAM-B), Neural cell adhesion molecule 1 (NCAM-1), and Mucin 4 (MUC-4). These ICDs are combined with diverse ECD interactions, including antibody-antigen and coiled-coil domains. It was found that these receptors behave in a modular fashion: the ICD dominates mechanical and morphological features of the resulting cell-cell interface. In contrast, the ECD independently determines connectivity—which cells “bond” to each other. This collection of synCAMs enables programming multicellular assemblies with defined patterning and reconfiguring of tissues organized by native adhesion. It has been further shown that synCAMs can engineer T cells with enhanced antigen-specific adhesion resistant to shear stress. The ability to control ECD recognition with versatile ICD signaling outputs reveals CAM evolutionary modularity and establishes synCAMs as a powerful toolkit for engineering cell-cell interaction networks.

### Example 10

### Cell Adhesion Molecule Intracellular Domains can be Fused to Novel Extracellular Domains to Generate Re-Directed Adhesion Receptors

Upon engagement of their ECDs, CAMs facilitate adhesion and cell junction organization by recruiting intracellular signaling proteins to the cell-cell interface. For example, cadherins, integrins, and intercellular adhesion molecule (ICAM) transduce signaling by binding adapter proteins that engage and reorganize the cytoskeleton (Barreiro et al., 2002; Kinashi, 2005; Yap and Kovacs, 2003). Here a large set of chimeric synthetic cell adhesion molecules (synCAMs) has been created by by fusing heterologous ECDs with native ICDs and assessed the adhesion strength and spatial organization of the resulting cell-cell interfaces. The synCAM interactions were assessed in L929 mouse fibroblasts, which lack strong intrinsic adhesion properties and were used as the background cell line in classic differential adhesion studies (Nose et al., 1988).

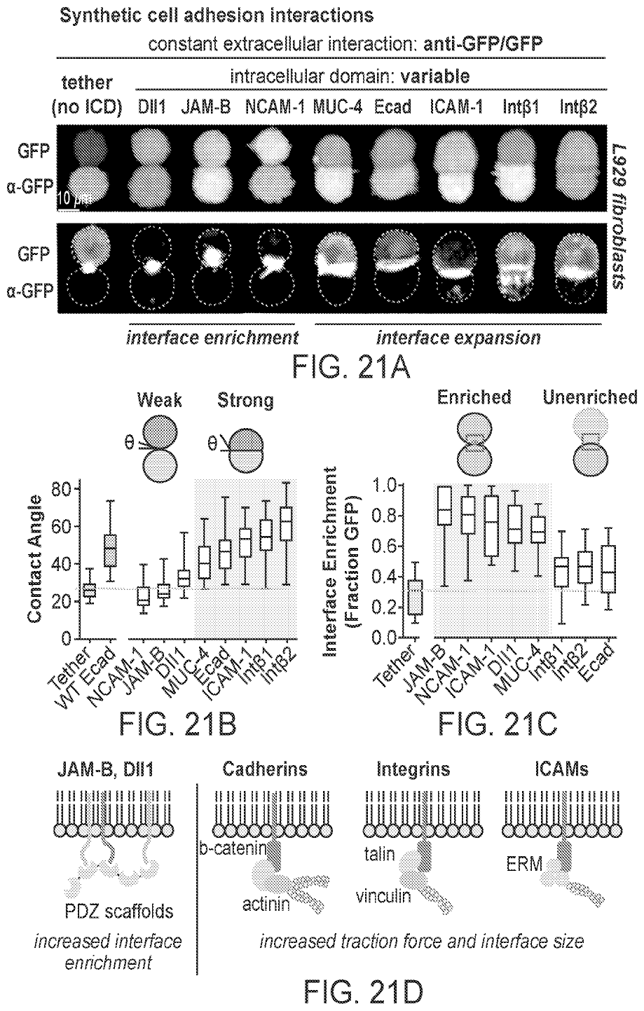

SynCAMs were constructed by fusing the transmembrane and ICD regions of endogenous CAMs with a heterologous extracellular interaction—in this case, GFP and its cognate nanobody (aGFP). ICD regions from the following natural adhesion proteins were used: Ecad, Int131, Int132, ICAM-1, Dll1, JAM-B, NCAM-1, and MUC-4. Versions of each synCAM were created with either GFP or a—GFP ECDs, and were stably expressed in L929 mouse fibroblasts. GFP and a—GFP cells with the same ICD were mixed in a flat bottom ultra low attachment (ULA) plate, and then imaged by confocal microscopy after 3 hours. Maximum projection images of representative cell-cell interface pairs for each class of synCAM are shown (FIG. 21A).

From these interaction pair images, adhesion strength was assessed by measuring contact angles of the relevant cell-cell interface. Spreading of a cell-cell interface is an equilibrium process that minimizes surface free energy, and thus the contact angle at the interface represents a measure of adhesion strength, similar to how contact angle can be used to measure surface tension of a liquid drop (Maitre et al., 2012; Winklbauer, 2015). Contact angles were quantified for the synCAM pairs shown in FIG. 21A (n=15-20 pairs; FIG. 21B) and compared to control cell pairs with either a tether (no ICD) or WT Ecad (homotypic interaction of full native protein). Several of the synCAMs, particularly those containing the ICD's from Ecad, ICAM-1, Int131, Int132 and MUC-4 showed contact angles significantly greater than the baseline tether interaction and comparable to that measured for native Ecad. In contrast, low contact angles comparable to the tether were observed with the NCAM-1, JAM-B, and Dll1 synCAMs. Thus, several synCAMs can achieve strong, native-like cell-cell adhesion (comparable to adhesion mediated by full-length Ecad), despite completely lacking their native extracellular interactions. Nonetheless, a distinct subset of ICD's did not yield synCAMs with strong adhesion.

In addition to modulating adhesion strength through the interface contact angle, CAM interactions can also facilitate the spatial organization of receptors at the cell-cell junction (Beutel et al., 2019; Wu et al., 2010). This ability to augment receptor organization within the membrane can in turn promote the activation of downstream signaling processes (Case et al., 2019; Su et al., 2016). To examine the contribution of the ICD to synCAM spatial enrichment, the amount of GFP fluorescent signal localized to the cell-cell interface was quantified relative to total cellular GFP (FIG. 21C) (n=15-20; non-interacting cells initially all show GFP uniformly distributed across the plasma membrane). Significant GFP enrichment at the cell-cell junction was observed for the NCAM-1, Dll1, JAM-B, ICAM-1, and MUC-4 relative to the tether contra_Other synCAMS (Intβ1, Intβ2, Ecad) showed moderate enhanced junctional enrichment. Although the degree of receptor enrich m

This study represents the first instance in which the behaviors of distinct CAM ICDs on the cell-cell interface can be directly compared, as a set of synCAMs that all use an identical extracellular interaction has been created. The synCAM ICDs facilitate two general properties of the interfaces they generate. Complementary SynCAMs with ICDs from MUC-4, Ecad, ICAM-1, Int131, and Int132 integrin form highly extended interfaces with strong adhesion, similar in strength to native adhesion molecules. In contrast, synCAMs with ICD's from JAM-B, Dll1, and NCAM-1 form smaller interfaces, lacking strong adhesion, but which result in the synCAMs organizing in a spatially enriched focus (often depleting the GFP construct from elsewhere in the cell). It is notable that the ICD's that result in strong and extended adhesion are known to recruit adapter proteins such as 0-catenin, talin, and ezrin-radixin-moesins (ERMs), which engage and reorganize the cytoskeleton (Barreiro et al., 2002; Kinashi, 2005; Yap and Kovacs, 2003). In contrast those that show the greatest focused enrichment are known to interact with intracellular PDZ domain scaffold proteins, such as mPdz and ZO-1, which play a role in receptor clustering, formation of tight junctions, and cell polarization (FIG. 21D) (Beutel et al., 2019; Sytnyk et al., 2006; Tetzlaff et al., 2018). Mutational analysis of the JAM-B, Int131, and ICAM-1 synCAMs (data not shown) support the key role of these intracellular interactions in determining interface properties.

### Example 11

### Different SynCAMs Activate Distinct Cytoskeletal Signaling Responses

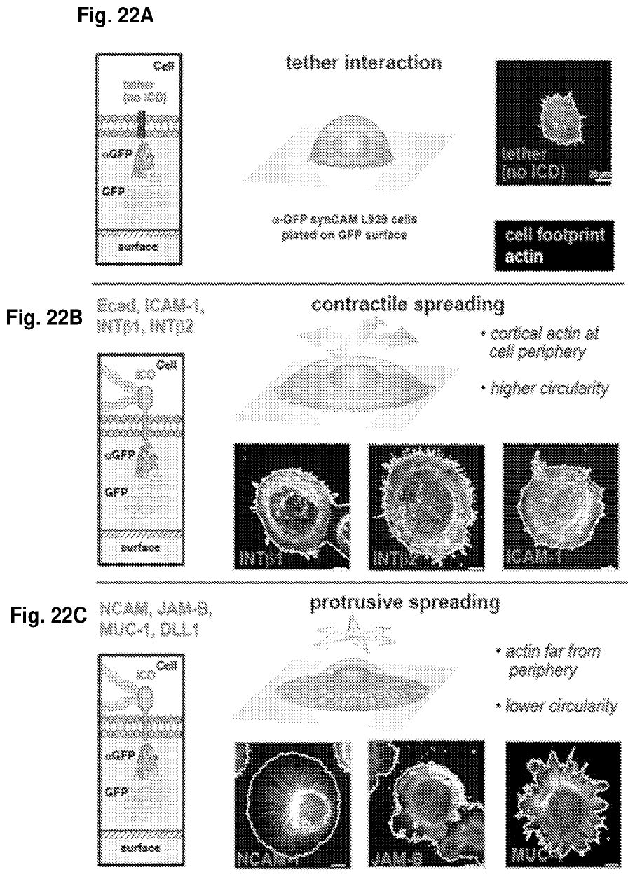

To further characterize the adhesion properties of modulating the synCAM ICD, the formation of different actin structures was classified using a cell spreading assay. α-GFP synCAM-expressing L929 cells were plated on a GFP-coated surface, then fixed and stained them with fluorescent phalloidin. Two distinct phenotypes were observed for the different synCAM cell lines (FIG. 22A). Cells expressing synCAMs with ICDs from ICAM-1, Int131, Int132, and Ecad uniformly spread on the surface and exhibit a dense band of cortical actin below the membrane at the periphery of the cell (FIG. 22B). However, the MUC-4, NCAM-1, and JAM-B ICDs result in nonuniform cell spreading on the GFP surface, with distinct cytoskeletal protrusions and the majority of actin far from the cell periphery (“fried egg” morphology) (FIG. 22C) Taken together, these results further support that the identity of the synCAM ICD directly determines the cytoskeletal properties of the adhesion interaction.

To characterize the physical adhesion properties of the different synCAMs more quantitatively, the rate and magnitude of cell spreading on GFP coated coverslips was measured (again using L929 cells expressing the oc-GFP synCAMs; data not shown). Prior reports indicate that cell spreading occurs in two phases: a fast phase (<10 min) based on initial adhesion, and a slow phase (10's of min to hours) in which increase in adhesive contact area is determined by reorganization of the actin cytoskeleton (Cuvelier et al., 2007). The spreading of L929 cells expressing the indicated ocGFP synCAMs (with different ICDs) on a GFP-coated surface over 75 minutes was measured (data not shown). Consistent with the cell-cell interface and phalloidin staining results, synCAMs with Ecad, ICAM-1, Intβ1, and Intβ2 ICDs exhibited strong slow-phase spreading on the GFP surface. Cell spreading behavior was highly distinct for cells expressing the tether construct (no ICD): the contact radius rapidly reached a plateau after the initial fast phase of spreading (<15 min), consistent with the absence of a second spreading phase involving major cytoskeletal reorganization.

### Example 12

### Intracellular Domain Signaling has a More Pronounced Impact on Cell-Cell Adhesion Preferences than Extracellular Domain Interaction Affinity

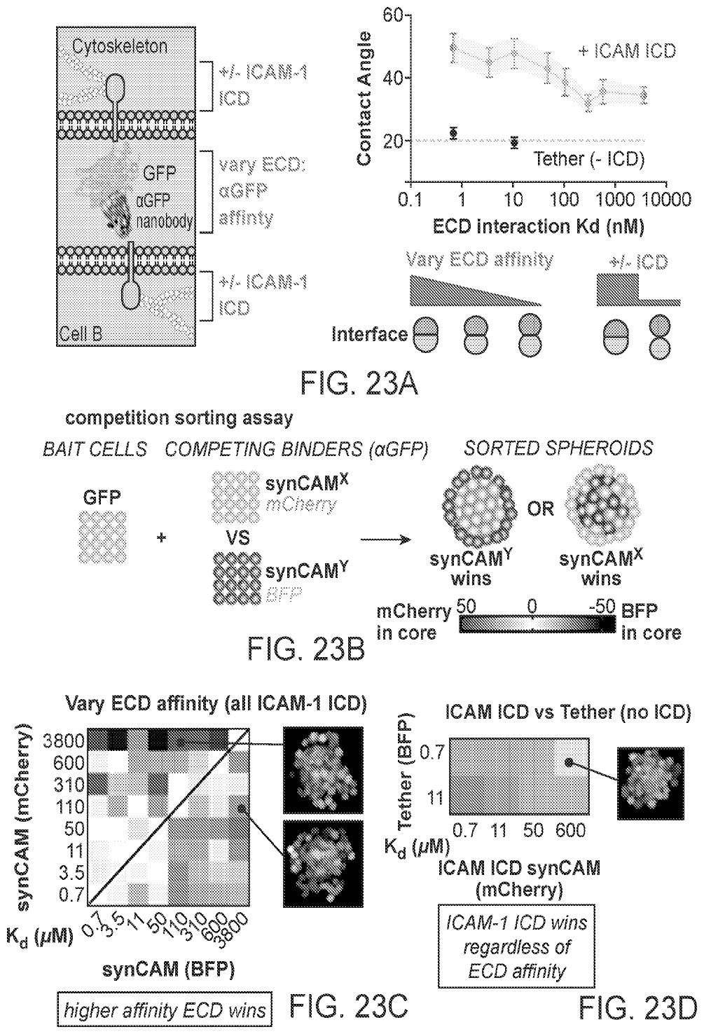

The interplay between intracellular and extracellular properties of synCAM function was explored by designing a series of aGFP-ICAM-1 synCAMs with varied extracellular affinity (using a series of aGFP nanobodies with variable Kd=0.7 nM to 3,000 nM). (Fridy et al., 2014) For comparison, aGFP-tether molecules (lacking ICDs) with variable extracellular affinities (Kd=0.7 and 11 nM) were constructed (Fridy et al., 2014). These aGFP synCAMs/tethers were stably expressed in L929 fibroblast cells, mixed with L929 cells expressing the complementary GFP-ICAM-1 synCAM (symmetric ICDs) in an ultra low adhesion plate, and imaged by confocal microscopy at t=3 h (FIG. 23A). Contact angles of individual cell-cell interfaces were quantified (n=20 error=95% CI). Despite spanning four orders of magnitude in ECD binding affinity, all aGFP ICAM-1 synCAMs exhibited greater average contact angles than the high affinity tethers. Even the lowest affinity ECD interactions, when combined with the ICAM-1 ICD, resulted in stronger adhesion than that produced by high affinity tethers (no ICD). Thus, the presence or absence of the ICAM-1 ICD plays a dominant role in determining adhesion strength.

Relative cell adhesion strength can also be functionally assayed by using a competition sorting assay, in which two different synCAM containing populations compete to interact with a complementary bait cell population (FIG. 23B). In this case the bait cell population express GFP synCAM with the ICAM-1 ICD. Two competing aGFP synCAM populations (differentially labelled with mCherry vs BFP) were added, to see which cells sort to the center via adhesion with the bait cells (differential sorting occurs in a liquid-like manner that minimizes surface free energy; (Foty and Steinberg, 2005; Steinberg, 1963; Winklbauer, 2015). The degree of sorting was quantified by measuring the average radial distance distribution of each cell type from the center of the assembly (d_{mCherry}-d_{BFP}) and is represented as a heat map (FIGS. 23C and 23D). The results from this population assay are consistent with the contact angle measurements of individual cell pairs (FIG. 23B). Cells expressing synCAMs with higher affinity ECDs bind tighter than those with lower affinity ECDs. However, when comparing synCAMs vs tethers, even synCAMs with low affinity ECDs outcompete the tethers, which lack the ICAM-1 ICD. These results indicate that the presence of the ICD is the dominant factor in determining high strength adhesion, while extracellular affinity provides a secondary level with which to tune adhesion strength.

### Example 13

### SynCAMs can be Engineered with Diverse Extracellular Binding Connectivity

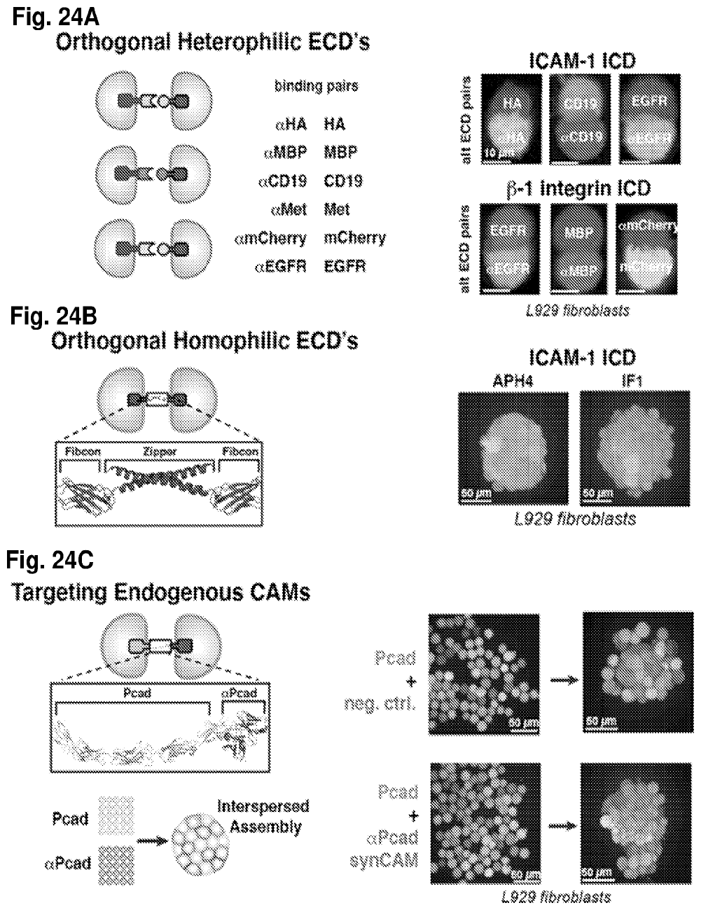

The programmability of synCAMs was investigated by engineering them with alternative, orthogonal extracellular interactions. Functional SynCAMs could be built with multiple distinct antibody-antigen binding pairs (FIG. 24A), including the following pairs: HA-tag/αHa scFv, maltose binding protein (MBP)/aMBP nanobody, B cell surface antigen CD19/aCD19 scFV, tyrosine-protein kinase Met (c-Met)/ac-Met nanobody, mCherry/amCherry nanobody, and epidermal growth factor receptor (EGFR)/aEGFR nanobody (FIG. 24A). The orthogonality of a subset of these receptor ECDs was confirmed using cell sorting assays in which synCAM pairs are mixed and characterized for their ability to sort from WT L929 cells (data not shown). Sorting to the core of the multicellular assembly is only observed in the case of matching antibody-antigen pairs. In addition, a single synCAM consisting of two sequential epitopes (HA-CD19), fused to the ICAM-1 TM and ICD (data not shown) was designed. Cells expressing this HA-CD19-ICAM-1 construct adhere to cells expressing either an aCD19 or aHA synCAM. Intb 1 TM and ICD domains were also generated with the MBP-aMBP, mCherry-amCherry, and EGFR-aEGFR ECD binding pairs (FIG. 24A).

Many endogenous CAMs function through an ECD that binds homophilically. For example, the homophilic specificity of cadherins such as Ncad and Pcad enable the differential sorting of tissue during development. (Halbleib and Nelson, 2006). A synCAM capable of mediating homophilic cell adhesion was therefore designed using an ECD in which a self-dimerizing leucine zipper was fused to a fibronectin fragment spacer domain (Fibcon). (Jacobs et al., 2012) The Aph4 leucine zipper (computationally designed) and the IF1 zipper (bovine ATPase inhibitor IF1) were utilized due to their antiparallel binding topologies, which was anticipated would sterically impair cis-inhibition on the surface of the same cell (Negron and Keating, 2014; Rhys et al., 2018). The Fibcon domain was included after direct fusion of the leucine zippers to the TMD proved unsuccessful, as anticipated that this spacer domain could further limit cis-interactions and provide additional separation from the juxtamembrane region. Homophilic cellular assembly was observed for both the Aph4 and IF1 ICAM-1 synCAMs (FIG. 24B).

Whether synCAMs could directly compete with a tissue held together by native adhesion molecules was also tested. A synCAM with an aPcad scFV fused to the ICAM-1 ICD was constructed, and whether L929 cells expressing this construct could intercalate with L929 cells expressing the native Pcad adhesion molecule was tested (FIG. 24C). These synthetic Pcad-targeting cells successfully incorporated into the Pcad homophilic assembly, while cells lacking the synCAM sorted to the exterior of the assembly. This result is consistent with the aPcad synCAM directly competing with the WT Pcad homophilic interaction for binding.

### Example 14

### De Novo Programming of Multi-Cellular Assembly with synCAMs

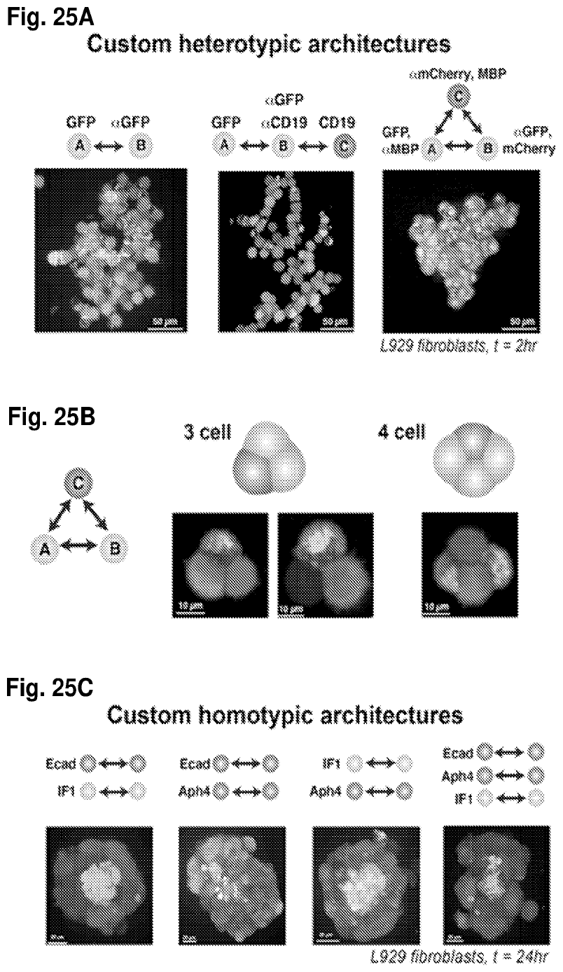

A fundamental goal in synthetic biology is to program the formation of novel multi-cellular tissues, de novo (Gartner and Bertozzi, 2009; Glass and Riedel-Kruse, 2018). This goal will require the capability to dictate specific cellular connectivities within a multicellular system. To determine whether spatial patterning can be rationally programmed using synCAMs, L929 cells were transduced with synCAMs and different extracellular binding domains. Sets of cells were plated in ultra low attachment (ULA) round bottom wells and imaged by confocal microscopy at t=2 hr (FIG. 25A). Assemblies with the following patterns were constructed: 1) two cell “A [symbol] B” alternating heterophilic interactions (generated by expression of a heterophilic GFP-aGFP synCAM pair in cells “A” and “B”); 2) three cell “A [symbol] B [symbol] C” bridging interactions (generated by expression of orthogonal synCAMs in cells “A” and “C”, and both complementary synCAMs in the bridging cell “B”); 3) three cell cyclic “A [symbol] B [symbol] C [symbol] A” interactions (generated by expression of two orthogonal synCAMs in each of cells “A”, “B”, and “C”). The resulting assemblies show that the cells organize into structures dictated by the synCAM defined cell-cell connectivities. As shown in the close-up images with low numbers of cells, the cyclic “A [symbol] B [symbol] C [symbol] A” interaction set can lead to the predicted types of minimal 3 and 4 cell multi-cell modules (FIG. 25B). synCAMs and synCAM combinations can be used to control the precise connectivities within complex multicell assemblies.

Different sets of cells expressing distinct orthogonal homophilic synCAMS were combined in order to program formation of structures with more complex segregated spatial compartments. Cells expressing three different orthogonal homotypic CAMs (WT Ecad, Aph4-ICAM-1, or IF1-ICAM-1) were mixed in different combinations and characterized by confocal microscopy after 24 hours (FIG. 25C). The individual cell populations show clear sorting. But what is most striking is the highly modular sorting behaviors that result. When cell types are mixed in a pairwise manner, it was observed that the IF1 synCAM sorts to the center vs Ecad introduction of synthetic adhesion could result in the remodeling and reconfiguration of tissue structures organized by native CAMs.

or Aph4 synCAM. The Ecad and Aph4 synCAM cells, sort into a two-lobed, bihemispheric structure. When all three cell types are mixed, it was observed that all of these relationships are maintained, yielding a structure with a Ecad/Aph4 synCAM barbell assembly, with IF1 cells at the core. These results show how the toolkit of orthogonal synCAMs can be used in modular and predictable way to build more complex, multi-compartment self-organizing structures.

### Example 15

### Modulating 3D Multicellular Sorting Through Incorporation of Synthetic Adhesion

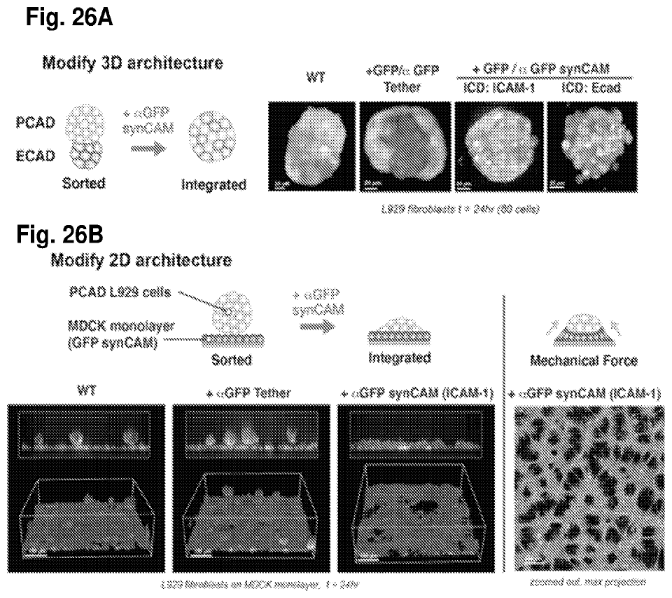

After demonstrating that synCAMs can be used to program novel structures, whether the introduction of synthetic adhesion could result in the remodeling and reconfiguration of tissue structures organized by native CAMs was examined. For example, previously generated WT Ecad and WT Pcad L929 cell lines that differentially sort into a bilobed assembly were examined (Toda et al., 2018). Whether introduction of synCAMs could remodel this structure was tested. Here complementary heterophilic synCAMs (with GFP/αGFP ECDs and either Ecad, ICAM-1 or no ICD) were expressed in the two cell lines (FIG. 26A). Expression of a weak heterotypic “tether” molecule converted the binodal assembly into a two layered structure. This two layered structure slightly increases the number of heterophilic contacts relative to the binodal assembly, but maintains compartmentalization mediated by the WT cadherins. Expression of the ICAM-1 synCAM, in contrast, converted the binodal structure into an interspersed structure in which the increased stability of the heterophilic interactions forces the two cell types into a single mixed cell compartment. Lasty, expression of the heterotypic Ecad synCAM also leads to integration of the two cell types. In this case the Ecad synCAM has two effects: it creates strong heterophilic binding and also has a dominant negative effect on WT cadherin signaling (thereby destabilizing the starting homophilic segregation). Along with modulating the sorting of WT Pcad and WT Ecad, an analogous experiment was carried out demonstrating the ability of synCAMs to augment the sorting of WT Pcad and WT Ncad (data not shown). Once again, a similar trend was observed whereby introduction of the synthetic heterophilic interaction augmented the compartmentalization of the WT P- and N-cadherins. Thus, synCAMs can clearly be used to predictably remodel and reconfigure multi-cell structures with native cell sorting.

### Example 16

### Remodeling Tissue Structure by Introducing Synthetic Adhesion Links

The interfacial organization of cells into 2D epithelial monolayers represents fundamental building block for diverse tissues and organs. Whether synCAMs could be used to modify or elaborate epithelial assemblies was tested. In this case Madin-Darby Canine Kidney cells (MDCKs) were used as a starting epithelial cell structure. MDCK cells were labeled with extracellular GFP as a ligand for engineered adhesion. Plated by themselves, the MDCK cells form a continuous epithelial layer at the bottom of the plate. L929 cells expressing Pcad were added, the L929 cells form spheroid clusters that sit above the MDCK epithelial layer with minimal interactions (FIG. 26B). Introduction of a synthetic adhesion interaction modifies this observed topography. When an aGFP tether interaction (no ICD) was added to the Pcad+L929 cells, they maintain their spheroid structure, but form a slightly more intimate and larger interface with the GFP+ MDCK cells. The introduction of a synthetic aGFP ICAM-1 interaction in the Pcad+L929 cells, however, causes the L929 cells to form a second layer of cells above the base layer of MDCK cells, leading to a structure similar to a stratified epithelium. Interesting, this two layer structure does not appear to lie completely flat—the strong Pcad homotypic interaction among the L929 cells, combined with the GFP/a-GFP interaction between the L929 and MDCK cells, appears to result in the formation of a network of L929 cells (right zoomed out image in FIG. 26B) that pulls up the surface bound MDCK cells in spaces between the network. Thus, synCAMs can be used to program the formation of more complex structures that build upon a 2D epithelial layer.

### CONCLUSIONS

### Modular Design of Adhesion Molecules Allows Evolution and Engineering of Diverse Types of Cell-Cell Interfaces and Assemblies

This work reveals that there is a vast potential for engineering diverse synthetic adhesion molecules that share the design principles of native adhesion molecules, but which specify new, specific, and orthogonal physical connectivities between cells. Although metazoans deploy a plethora of cell adhesion molecules to mediate diverse cellular interactions and tissue assembly, many more novel interfaces remain untapped by evolution. The synCAM design strategy incorporates two central modes for controlling synthetic adhesion. First, the extracellular interaction domain determines the nature of molecular recognition and cell-cell connectivity (“bonding”). Binding can be either homophilic or heterophilic, and employing a programmable recognition domain such as an antibody fragment customizes both the identity and affinity of the target ligand interaction. Second, the intracellular domain dictates the signaling network that activates upon ECD engagement and determines the cellular mechanics of the interaction. Domains such as ICAM-1, Ecad, and β-integrins lead to cytoskeletal reorganization that favors tight and extended cell-cell interface formation, while JAM-B and NCAM-1 greatly enhance receptor enrichment without formation of a tight interface. This toolkit can thus alter both cell-cell connectivity and the resulting cytoskeletal organization at the interface.

The broad spectrum of adhesion ICDs amenable to chimeric engineering demonstrates that intracellular domain function is often relatively independent of the endogenous extracellular recognition mechanism. It is noteworthy that the simple extracellular interactions utilized in this work do not match the higher regulatory sophistication of many natural adhesion ECDs. For example, cadherin ECDs cooperatively oligomerize in cis, while integrin ECDs transition from a closed to open conformation. (Hynes, 2002; Luo and Springer, 2006; Rubinstein et al., 2015; Wu et al., 2010) Nonetheless, despite normally functioning with sophisticated ECDs, the ICD's from these adhesion proteins direct assembly of a similar cell-cell interface when coupled to simpler chimeric ECDs. One can find numerous examples of such modularity in natural evolution. Proteins with Cadherin ECDs are found in choanoflagellates (the closest single cell relatives to metazoans), but there lack the metazoan ICDs (Abedin and King, 2008; King et al., 2003). These proteins may have been used by choanoflagellates to bind food or substrates rather than for cell-cell adhesion. In addition, pathogenic bacteria can plug into adhesion systems, as in the case of Listeria monocytogenes, which crosses the host intestinal barrier using a protein that heterotypically engages the normally homotypic host adhesion protein Ecad (Pizarro-Cerda et al., 2012). Despite this ability to function with relatively simple ECDs, future efforts could incorporate complexities found in WT CAMs to enable synCAMs with cooperative or conditional recognition.

Our findings illustrate the dominant character of cytoskeletal signaling in dictating multicellular assembly. Although the ECD specifies interaction partners and fine tunes strength, the ICD defines the resulting cell-cell interaction through engaging systems such as the cytoskeleton. It was observed for cell-cell pairs that ICDs such as those from Ecad, ICAM-1, Intrβ1, and Intrβ2 result in a far tighter adhesion interface than could be provided solely by the ECD interaction. This is consistent with prior reports that intracellular signaling effects on cortical tension are the primary factor in determining CAM adhesion strength. (Maitre et al., 2012; Winklbauer, 2015)

### REFERENCES

Abedin, M., and King, N. (2008). The premetazoan ancestry of cadherins. Science 319, 946-948.
Bakalar, M. H., Joffe, A. M., Schmid, E. M., Son, S., Podolski, M., and Fletcher, D. A. (2018). Size-Dependent Segregation Controls Macrophage Phagocytosis of Antibody-Opsonized Targets. Cell 1 74, 131-142.e13.
Barreiro, O., Yanez-Mo, M., Serrador, J. M., Montoya, M. C., Vicente-Manzanares, M., Tejedor, R., Furthmayr, H., and Sanchez-Madrid, F. (2002). Dynamic interaction of VCAM-1 and ICAM-1 with moesin and ezrin in a novel endothelial docking structure for adherent leukocytes. J. Cell Biol. 157, 1233-1245.
Beutel, O., Maraspini, R., Pombo-Garcia, K., Martin-Lemaitre, C., and Honigmann, A. (2019). Phase separation of zonula occludens proteins drives formation of tight junctions. Cell 179,923-936.el 1.
Case, L. B., Ditlev, J. A., and Rosen, M. K. (2019). Regulation of transmembrane signaling by phase separation. Annu. Rev. Biophys. 48, 465-494.
Cavallaro, U., and Dejana, E. (2011). Adhesion molecule signalling: not always a sticky business. Nat. Rev. Mol. Cell Biol. 12, 189-197.
Cuvelier, D., Théry, M., Chu, Y.-S., Dufour, S., Thiery, J.-P., Bornens, M., Nassoy, P., and Mahadevan, L. (2007). The universal dynamics of cell spreading. Curr. Biol. 17, 694-699.
Ebnet, K., Suzuki, A., Ohno, S., and Vestweber, D. (2004). Junctional adhesion molecules (JAMs): more molecules with dual functions? J. Cell Sci. 117, 19-29.
Foty, R. A., and Steinberg, M. S. (2005). The differential adhesion hypothesis: a direct evaluation. Dev. Biol. 278, 255-263.
Fridy, P. C., Li, Y., Keegan, S., Thompson, M. K., Nudelman, I., Scheid, J. F., Oeffinger, M., Nussenzweig, M. C., Fenyo, D., Chait, B. T., et al. (2014). A robust pipeline for rapid production of versatile nanobody repertoires. Nat. Methods 11, 1253-1260.
Gartner, Z. J., and Bertozzi, C. R. (2009). Programmed assembly of 3-dimensional microtissues with defined cellular connectivity. Proc. Natl. Acad. Sci. USA 106, 4606-4610.
Geiger, B., Salomon, D., Takeichi, M., and Hynes, R. O. (1992). A chimeric N-cadherin/beta 1-integrin receptor which localizes to both cell-cell and cell-matrix adhesions. J. Cell Sci. 103 (Pt 4), 943-951.
Glass, D. S., and Riedel-Kruse, I. H. (2018). A Synthetic Bacterial Cell-Cell Adhesion Toolbox for Programming Multicellular Morphologies and Patterns. Cell 174, 649-658.e16.
Halbleib, J. M., and Nelson, W. J. (2006). Cadherins in development: cell adhesion, sorting, and tissue morphogenesis. Genes Dev. 20, 3199-3214.
Harjunpää, H., Llort Asens, M., Guenther, C., and Fagerholm, S. C. (2019). Cell adhesion molecules and their roles and regulation in the immune and tumor microenvironment. Front. Immunol. 10, 1078.
Honig, B., and Shapiro, L. (2020). Adhesion Protein Structure, Molecular Affinities, and Principles of Cell-Cell Recognition. Cell 181, 520-535.
Hynes, R. O. (2002). Integrins: bidirectional, allosteric signaling machines. Cell 110, 673-687.
Jacobs, S. A., Diem, M. D., Luo, J., Teplyakov, A., Obmolova, G., Malia, T., Gilliland, G. L., and O'Neil, K. T. (2012). Design of novel FN3 domains with high stability by a consensus sequence approach. Protein Eng Des Sel 25, 107-117.
June, C. H., O'Connor, R. S., Kawalekar, O. U., Ghassemi, S., and Milone, M. C. (2018). CAR T cell immunotherapy for human cancer. Science 359, 1361-1365.
Kinashi, T. (2005). Intracellular signalling controlling integrin activation in lymphocytes. Nat. Rev. Immunol. 5, 546-559.
King, N., Hittinger, C. T., and Carroll, S. B. (2003). Evolution of key cell signaling and adhesion protein families predates animal origins. Science 301, 361-363.
LaFlamme, S. E., Thomas, L. A., Yamada, S. S., and Yamada, K. M. (1994). Single subunit chimeric integrins as mimics and inhibitors of endogenous integrin functions in receptor localization, cell spreading and migration, and matrix assembly. J. Cell Biol. 126, 1287-1298.
Ley, K., Laudanna, C., Cybulsky, M. I., and Nourshargh, S. (2007). Getting to the site of inflammation: the leukocyte adhesion cascade updated. Nat. Rev. Immunol. 7,678-689.
Lilienbaum, A., Reszka, A. A., Horwitz, A. F., and Holt, C. E. (1995). Chimeric integrins expressed in retinal ganglion cells impair process outgrowth in vivo. Mol. Cell. Neurosci. 6, 139-152.
Luo, B.-H., and Springer, T. A. (2006). Integrin structures and conformational signaling. Curr. Opin. Cell Biol. 18, 579-586.
Maître, J.-L., Berthoumieux, H., Krens, S. F. G., Salbreux, G., Jillicher, F., Paluch, E., and Heisenberg, C.-P. (2012). Adhesion functions in cell sorting by mechanically coupling the cortices of adhering cells. Science 338, 253-256.
Morrissey, M. A., Williamson, A. P., Steinbach, A. M., Roberts, E. W., Kern, N., Headley, M. B., and Vale, R. D. (2018). Chimeric antigen receptors that trigger phagocytosis. Elife 7.
Morsut, L., Roybal, K. T., Xiong, X., Gordley, R. M., Coyle, S. M., Thomson, M., and Lim, W. A. (2016). Engineering customized cell sensing and response behaviors using synthetic notch receptors. Cell 164, 780-791.
Negron, C., and Keating, A. E. (2014). A set of computationally designed orthogonal antiparallel homodimers that expands the synthetic coiled-coil toolkit. J. Am. Chem. Soc. 136, 16544-16556.
Newick, K., O'Brien, S., Moon, E., and Albelda, S. M. (2017). CAR T cell therapy for solid tumors. Annu. Rev. Med. 68, 139-152.
Nose, A., Nagafuchi, A., and Takeichi, M. (1988). Expressed recombinant cadherins mediate cell sorting in model systems. Cell 54, 993-1001.
Pizarro-Cerda, J., Kilhbacher, A., and Cossart, P. (2012). Entry of Listeria monocytogenes in mammalian epithelial cells: an updated view. Cold Spring Harb. Perspect. Med. 2.
Rhys, G. G., Wood, C. W., Lang, E. J. M., Mulholland, A. J., Brady, R. L., Thomson, A. R., and Woolfson, D. N. (2018). Maintaining and breaking symmetry in homomeric coiled-coil assemblies. Nat. Commun. 9, 4132.
Rubinstein, R., Thu, C. A., Goodman, K. M., Wolcott, H. N., Bahna, F., Mannepalli, S., Ahlsen, G., Chevee, M., Halim, A., Clausen, H., et al. (2015). Molecular logic of neuronal self-recognition through protocadherin domain interactions. Cell 163, 629-642.
Sackstein, R., Schatton, T., and Barthel, S. R. (2017). T-lymphocyte homing: an underappreciated yet critical hurdle for successful cancer immunotherapy. Lab. Invest. 97, 669-697.
Sockolosky, J. T., Trotta, E., Parisi, G., Picton, L., Su, L. L., Le, A. C., Chhabra, A., Silveria, S. L., George, B. M., King, I. C., et al. (2018). Selective targeting of engineered T cells using orthogonal IL-2 cytokine-receptor complexes. Science 359, 1037-1042.
Steinberg, M. S. (1963). Reconstruction of tissues by dissociated cells. Some morphogenetic tissue movements and the sorting out of embryonic cells may have a common explanation. Science 141, 401-408.
Su, X., Ditlev, J. A., Hui, E., Xing, W., Banjade, S., Okrut, J., King, D. S., Taunton, J., Rosen, M. K., and Vale, R. D. (2016). Phase separation of signaling molecules promotes T cell receptor signal transduction. Science 352, 595-599.
Sun, Z., Guo, S. S., and Fassler, R. (2016). Integrin-mediated mechanotransduction. J. Cell Biol. 215, 445-456.
Sytnyk, V., Leshchyns'ka, I., Nikonenko, A. G., and Schachner, M. (2006). NCAM promotes assembly and activity-dependent remodeling of the postsynaptic signaling complex. J. Cell Biol. 174, 1071-1085.
Tetzlaff, F., Adam, M. G., Feldner, A., Moll, I., Menuchin, A., Rodriguez-Vita, J., Sprinzak, D., and Fischer, A. (2018). MPDZ promotes DLL4-induced Notch signaling during angiogenesis. Elife 7.
Toda, S., Blauch, L. R., Tang, S. K. Y., Morsut, L., and Lim, W. A. (2018). Programming self-organizing multicellular structures with synthetic cell-cell signaling. Science 361, 156-162.
Toda, S., Frankel, N. W., and Lim, W. A. (2019). Engineering cell-cell communication networks: programming multicellular behaviors. Curr. Opin. Chem. Biol. 52, 31-38.
Todhunter, M. E., Jee, N.Y., Hughes, A. J., Coyle, M. C., Cerchiari, A., Farlow, J., Garbe, J. C., LaBarge, M. A., Desai, T. A., and Gartner, Z. J. (2015). Programmed synthesis of three-dimensional tissues. Nat. Methods 12, 975-981.
Winklbauer, R. (2015). Cell adhesion strength from cortical tension—an integration of concepts. J. Cell Sci. 128, 3687-3693.
Wu, Y., Jin, X., Harrison, O., Shapiro, L., Honig, B. H., and Ben-Shaul, A. (2010). Cooperativity between trans and cis interactions in cadherin-mediated junction formation.
Proc. Natl. Acad. Sci. USA 107, 17592-17597.
Yap, A. S., and Kovacs, E. M. (2003). Direct cadherin-activated cell signaling: a view from the plasma membrane. J. Cell Biol. 160, 11-16.
Young, B. A., Taooka, Y., Liu, S., Askins, K. J., Yokosaki, Y., Thomas, S. M., and Sheppard, D. (2001). The cytoplasmic domain of the integrin alpha9 subunit requires the adaptor protein paxillin to inhibit cell spreading but promotes cell migration in a paxillin-independent manner Mol. Biol. Cell 12, 3214-3225.

1. A fusion protein comprising:
(i) an extracellular binding domain comprising a first binding moiety that is capable of specific binding to a second binding moiety;
(ii) one or more transmembrane domains; and
(iii) an intracellular domain that is capable of signaling to the cytoskeleton of the cell upon binding of the first binding moiety to the second binding moiety,
wherein the extracellular binding domain and the intracellular binding domain of the fusion protein are not from the same native cell adhesion molecule.

2. The fusion protein of claim 1, wherein the first binding moiety is a scFv or nanobody.

3. The fusion protein of claim 1, wherein:
the intracellular domain is not from an engulfment receptor;
the intracellular domain does not contain a co-stimulatory domain or intracellular T-cell activation domain (ITAM);
the fusion protein, when expressed in a cytotoxic immune cell or stem cell, does not induce phagocytosis of the cell when it binds to the second binding moiety that is on another cell or scaffold;
the fusion protein, when expressed in cytotoxic immune cell, does not activate the cell when it binds to the second binding moiety that is on another cell or scaffold; and
the extracellular binding domain of the fusion protein is not an extracellular binding domain of a native cell adhesion molecule.

4. The fusion protein of claim 1, wherein the intracellular domain is an intracellular domain of a cell adhesion molecule selected from Table 1, or a variant thereof that retains the ability to engage with the cytoskeleton.

5. The fusion protein of claim 1, wherein the fusion protein, when expressed in a mammalian cell, engages with the cytoskeleton of the cell when it binds to the second binding moiety that is on another cell or scaffold.

6. The fusion protein of claim 1, wherein the first binding moiety is capable of specifically binding:
(a) to a naturally-occurring protein expressed on the surface of a partner cell;
(b) to a non-naturally-occurring protein expressed on the surface of a partner cell;
(c) to a scaffold molecule or material bearing the cognate ligand, including natural or unnatural extracellular matrix molecules or hydrogels;
(d) to a partner cell via a homophilic interaction;
(e) to partner cells via a heterophilic interaction;
(f) to multiple partner cells or substrates via a multivalent interaction;
(g) to a partner cell or substrate via a chemically inducible interaction;
(h) to a partner cell or substrate via light- or protease-activated interaction; and/or
(i) to multiple partner cells or substrates via tandem recognition domains.

7. A nucleic acid encoding a fusion protein of claim 1.

8. A mammalian cell comprising the nucleic acid of claim 7.

9. The cell of claim 8, wherein the fusion protein does not induce phagocytosis of the mammalian cell when it binds to the second binding moiety that is on another cell or scaffold.

10. The cell of claim 8, wherein the cell is an immune cell selected from a T cell and a natural killer (NK) cell and, optionally, a macrophage.

11. The cell of claim 10, wherein the fusion protein, when expressed in the immune cell, does not activate the cell or induce phagocytosis when it binds to the second binding moiety that is on another cell or scaffold.

12. The cell of claim 8, wherein the cell is a stem cell.

13. A composition comprising a recombinant cell of claim 12 and a growth medium.

14. The composition of claim 13, wherein the composition further comprises a second cell, wherein the recombinant cell and the second cell adhere to each other via an interaction that requires binding of the first binding moiety to the second binding moiety.

15. The composition of claim 14, wherein the recombinant cell and the second cell adhere to each other directly or indirectly via binding of the first binding moiety to a second binding moiety that is on the surface of second cell or scaffold.

16. A method for altering the binding characteristics of a cell, comprising introducing a nucleic acid encoding a fusion protein of claim 1 into the cell, wherein said introducing results in expression of the fusion protein and alteration of the binding characteristics of the cell.

17. The method of claim 16, wherein:
(a) the extracellular binding domain binds to a tissue-specific surface molecule and expression of the fusion protein in the cell results in a longer residency in a selected tissue relative to the same cell without the fusion protein;
(b) the extracellular binding domain binds to a disease-specific surface molecule and expression of the fusion protein in the cell results in a longer residency in a diseased tissue relative to the same cell without the fusion protein;
(c) the extracellular binding domain binds to a molecule on the surface of a target cell and
i increases the formation of multicellular tissues with a defined structure in vitro or in vivo, controls cell sorting based on differential adhesion strengths;
ii. controls autonomous sorting of cells based on differential adhesion strengths;
iii. directs the assembly of an organoid in a disease model;
iv. directs the assembly of an organ or tissue;
v. directs regeneration of a tissue or organ in vivo;
vi. assists in the formation of epithelial-like cell assemblies; or
vii. directs specific cell-cell connectivities, including multicell circuit/communication systems, including neuronal and endocrine multi-cell systems;
(d) enhances, inhibits or modulates the function of other cell-cell interaction molecules and use in engineering multi-antigen target AND or NOT gates;
(e) abrogates disfunctional adhesion; or
(f) directs or enhances phagocytosis of cognate target cells.

18. A method of treatment, comprising:
administering a cell of claim 8 to a subject, wherein the first binding moiety of the recombinant cell recognizes an antigen on a target cell in the subject and the recombinant cell adheres to the target cell in the subject in vivo.

19. The method of claim 18, wherein the antigen is a disease-specific or tissue-specific antigen.

20. A method for adhering a cell to a scaffold, comprising:
combining a cell of any of claim 8 with a scaffold, wherein the first binding moiety of the engineered cell adhesion molecule binds to the scaffold and the recombinant cell adheres to the scaffold.
